(19) United States
(12) Patent Application Publication
(10) Pub. No.: US 2022/0273805 A1
(43) Pub. Date: Sep. 1, 2022

(54) Cannabinoid Conjugate Molecules

(71) Applicant: Diverse Biotech, Inc., Orlando, FL (US)

(72) Inventors: Paul Hershberger, Orlando, FL (US); Philip Arlen, Orlando, FL (US)

(21) Appl. No.: 17/622,382

(22) PCT Filed: Jun. 24, 2020
(86) PCT No.: PCT/US20/39234

§ 371 Date: Dec. 23, 2021

Related U.S. Application Data
(60) Provisional application No. 62/960,066, filed on Jan. 12, 2020.
(60) Provisional application No. 62/899,674, filed on Sep. 12, 2019.
(60) Provisional application No. 62/865,699, filed on Jun. 24, 2019.

(51) Int. Cl. A61K 47/55; A61K 47/54; A61K 45/06

(52) U.S. Cl. CPC A61K 47/55; A61K 45/06; A61K 47/545

(57) ABSTRACT

This disclosure provides multifunctional conjugate molecules in which at least one therapeutic agent is covalently attached to a cannabinoid by means of a linker. The disclosed conjugate molecules are designed to deliver therapeutic benefits of components of the conjugate molecules and can be used to treat cancer and other disorders.

Each reference cited in this disclosure is incorporated by reference herein in its entirety.

### TECHNICAL FIELD

This disclosure relates generally to multifunctional therapeutics.

### DETAILED DESCRIPTION

This disclosure describes multifunctional conjugate molecules comprising at least one therapeutic agent component and at least one cannabinoid component covalently attached by a linker:

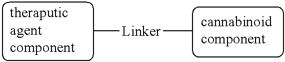

In contrast to traditional prodrugs, embodiments of the disclosed conjugate molecules are designed to deliver more than one therapeutic benefit via more than one mechanism of action; this is achieved when the covalent binding of the therapeutic agent component to its target enables the release of the cannabinoid at or near the site of the therapeutic agent's action, which can then effect a second therapeutic benefit. That is, these conjugate molecules are designed to deliver the therapeutic benefits of each of their components. In other embodiments, the therapeutic agent component and the cannabinoid component are released to provide their respective therapeutic benefits via functionality of the linker.

For example, the formation of reactive oxygen species (ROS) is a by-product of the normal process of respiration in an oxygen-rich environment (Storz & Imlay, Curr. Opin. Microbiol. 2, 188-94, 1999). There is significant evidence in the literature for the role endogenous ROS plays in mutagenesis, as well as its contribution to the mutational burden experienced by microbes during periods of oxidative stress (reviewed in Dwyer et al., Curr. Opin. Microbiol. 12, 482-89, 2009). In fact, bacteria have evolved several enzymatic mechanisms to combat ROS toxicity (Imlay, Ann. Rev. Biochem. 77, 755-76, 2008).

ROS are generated intracellularly and include superoxide (O₂.⁻), hydrogen peroxide (H₂O₂), and highly destructive hydroxyl radicals (OH.). The species O₂.⁻ and H₂O₂ can be enzymatically eradicated by the activity of superoxide dismutases and catalases/peroxidases, respectively.

Excess intracellular levels of ROS cause damage to proteins, nucleic acids, lipids, membranes, and organelles, which can lead to activation of cell death processes such as apoptosis. Apoptosis is a tightly regulated and highly conserved process of cell death during which a cell undergoes self-destruction (Kerr et al., Br. J. Cancer 26, 239-57, 1972). Apoptosis can be triggered by a variety of extrinsic and intrinsic signals, including ROS (reviewed in Redza-Dutordoir & Averill-Bates, Biochem. Biophys. Acta 1863, 2977-92, 2016). Exposure to xenobiotics such as antibiotics and chemotherapeutic drugs can also trigger apoptosis, and is often mediated by ROS.

Cannabinoids have demonstrated their ability to promote ROS production. Cannabidiol (CBD) is a non-toxic and non-psychoactive cannabinoid that has been shown to have anti-tumor activity in multiple cancer types (Massi et al., J. Pharmacol. Exp. Ther. 308, 838-45, e-pub 2003). Activation of the endogenous cannabinoid type 1 (CB1) and type 2 (CB2) receptors has been shown to inhibit tumor progression (Velasco et al., Nat. Rev. Cancer 12, 436-44, 2012). CBD has been reported to inhibit human GBM viability in culture, an effect that was reversed in the presence of the ROS scavenger α-tocopherol/vitamin E (Velasco et al., 2012).

CBD-dependent production of ROS has been shown to accompany a reduction in glutathione (Massi et al., Cell. Mol. Sci. 63, 2057-66, 2006), an important anti-oxidant that prevents damage to cellular components by ROS. The source of CBD-dependent stress in part originated in the mitochondria and led to activation of multiple caspases involved in intrinsic and extrinsic pathways of apoptosis. Further studies analyzing CBD-treated GBM tumor tissue revealed that inhibition of lipoxygenase signaling played a role in CBD anti-tumor activity (McAllister et al., J. Neuroimmune Pharmacol. 10, 255-67, 2015). In addition, the indirect modulation of the endocannabinoid system by CBD may be attributed to the observed anti-tumor activity.

Cannabigerol (CBG) is another non-psychotropic cannabinoid that interacts with specific targets involved in carcinogenesis and has shown potent anti-tumor activity (Guindon & Hohmann, Br. J. Pharmacol. 163, 1447-63, 2011). Mechanistically, CBG, similar to CBD, appears to influence the inflammatory microenvironment that is important in the initiation and progression of cancer (Mantovani et al., Nature 454, 436-44, 2008; Solinas et al., Cancer Metastasis Rev. 29, 243-48, 2010). Moreover, CBG was also able to exert pro-apoptotic effects by selectively increasing ROS production in colorectal cancer cells but not in healthy colonic cells (Borrelli et al., Carcinogenesis 35, 2787-97, 2014).

Conjugate Molecules

Conjugate molecules comprise at least one therapeutic agent component covalently linked, directly or via a linker, to at least one cannabinoid component.

In some embodiments, a therapeutic agent component is covalently attached directly to a hydroxy or carboxylic acid group of a cannabinoid component. In some embodiments, cannabinoid conjugate components comprise a therapeutic agent component and a cannabinoid component attached by means of a linker which is covalently attached at one end to the therapeutic agent component and at the other end to a hydroxy or carboxylic acid group of the cannabinoid component. In some embodiments, the hydroxy group is an “aromatic hydroxy group;” i.e., a hydroxy group bonded directly to an aromatic hydrocarbon. In some embodiments, the hydroxy group is an “aliphatic hydroxy group;” i.e., a hydroxy group bound to a carbon that is not part of an aromatic ring.

In some embodiments, conjugate molecules contain only one therapeutic agent component. In other embodiments, for example, when a cannabinoid component has at least two hydroxy groups, or at least one hydroxy group and at least one carboxylic acid group, or at least two carboxylic acid groups, conjugate molecules can contain two or more therapeutic agent components, which can be the same or different.

In some embodiments, in which therapeutic agent components are attached via a linker, the two or more linkers can be the same or different and, independently, the two or more therapeutic agent components can be the same or different. Also independently, when a cannabinoid component contains two or more hydroxy groups, the two or more hydroxy groups can be aliphatic or the two or more hydroxy groups can be aromatic, or, for example, a first hydroxy group can be aliphatic and a second hydroxy group can be aromatic.

In some embodiments using particular types of linkers described below, conjugate molecules can contain two therapeutic agent components which are both attached to a single linker. The two therapeutic agent components can be the same or different.

In some embodiments, a conjugate molecule can contain an additional cannabinoid component.

Conjugate molecules can have one or more centers of asymmetry and can therefore be prepared either as a mixture of isomers (e.g., a racemic or diasteromeric mixture) or in an enantiomerically or diasteromerically pure form. Such forms include, but are not limited to, diastereomers, enantiomers, and atropisomers. Conjugate molecules can also include alkenes and can therefore be prepared either as a mixture of double bond isomers or independently as either an E or Z isomer. Isotopic variants of conjugate molecules can also be prepared.

Conjugate molecules can form salts. “Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth metal ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Further examples of pharmaceutically acceptable salts include those listed in Berge et al., Pharmaceutical Salts, J. Pharm. Sci. 1977 January; 66(1):1-19.

### Definitions

The following definitions apply to the descriptions of the “Therapeutic Agent Component(s)” and “Linkers” in the sections below and to the descriptions of “Group One Substituents” and “Group Two Substituents.”

“C1-C3 linear or branched alkyl” means “methyl, ethyl, propyl, and isopropyl.”

“C1-C8 linear or branched alkyl” means “methyl, ethyl, C3, C4, C5, C6, C7, and C8 linear alkyl and C3, C4, C5, C6, C7, and C8 branched alkyl.”

“C1-C3 linear or branched heteroalkyl” means “a linear or branched heteroalkyl containing 1, 2, or 3 carbon atoms.”

“C1-C8 linear or branched heteroalkyl” means “each of a C1, C2, C3, C4, C5, C6, C7, and C8 linear heteroalkyl and C1, C2, C3, C4, C5, C6, C7, and C8 branched heteroalkyl.”

“C1-C12 linear or branched heteroalkyl” means each of a C1, C2, C3, C4, C5, C6, C7, C8, C9, C10, C11, and C12 linear heteroalkyl and C1, C2, C3, C4, C5, C6, C7, C8, C9, C10, C11, and C12 branched heteroalkyl.”

“C1-C24 linear or branched heteroalkyl” means each of a C1, C2, C3, C4, C5, C6, C7, C8, C9, C10, C11, C12, C13, C14, C15, C16, C17, C18, C19, C20, C21, C22, C23, and C24 linear heteroalkyl and C1, C2, C3, C4, C5, C6, C7, C8, C9, C10, C11, C12, C13, C14, C15, C16, C17, C18, C19, C20, C21, C22, C23, and C24 branched heteroalkyl.”

“C1-C6 linear or branched alkoxyl” means “a linear or branched alkoxyl containing 1, 2, 3, 4, 5, or C carbon atoms.”

“C1-C6 linear or branched alkylamino” means “a linear or branched alkylamino containing 1, 2, 3, 4, 5, or 6 carbon atoms.”

“C1-C6 linear or branched dialkylamino” means “each linear or branched dialkylamino in which each alkyl independently contains 1, 2, 3, 4, 5, or 6 carbon atoms.”

“6-10-membered aromatic” means “each of a 6-, 7-, 8-, 9-, and 10-membered aromatic.”

“5- to 10-membered heteroaromatic” means “each of a 6-, 7-, 8-, 9-, and 10-membered heteroaromatic.”

“3- to 9-membered cycloheteroalkyl” means “each of a 3-, 4-, 5-, 6-, 7-, 8-, and 9-membered cycloheteroalkyl.

“C3-C6 cycloalkyl” means “C3. C4, C5, and C6 cycloalkyl.”

“Halide” means “Cl, Br, and I.”

“Group One Substituents” is a group of substituents consisting of.
(a) —OH;
(b) —NH₂;
(c) ═O;
(d) ═S;
(e) ═NR7, where R7 is H or is C1-C3 linear or branched alkyl or C1-C3 linear or branched heteroalkyl comprising an O, N, or S atom;
(f) —C(O)OR4, wherein R4 is H or C1-C3 linear or branched alkyl;
(g) —C(O)NR5R6, wherein R5 and R6 independently are H or C1-C6 linear or branched alkyl;
(h) halide;
(i) C1-C6 linear or branched alkoxyl;
(j) C1-C6 linear or branched alkylamino;
(k) C1-C6 linear or branched dialkylamino;
(l) 6- to 10-membered aromatic, optionally substituted with 1, 2, 3, or 4 substituents independently selected from
   (i) phenyl;
   (ii) halide;
   (iii) cyano;
   (iv) C1-C6 linear or branched alkyl, optionally substituted with
      (1) 1, 2, 3, 4, 5, 6, 7, 8, or 9 fluorine atoms; and/or
      (2) 1, 2, or 3 substituents independently selected from the Group Two Substituents; and
   (v) C1-C6 linear or branched heteroalkyl containing 1, 2, or 3 atoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group Two Substituents;
(m) 5- to 10-membered heteroaromatic, optionally substituted with 1, 2, 3, or 4 substituents independently selected from
   (i) phenyl;
   (ii) halide;
   (iii) cyano;
   (iv) C1-C6 linear or branched alkyl, optionally substituted with
      (1) 1, 2, 3, 4, 5, 6, 7, 8, or 9 fluorine atoms; and/or
      (2) 1, 2, or 3 substituents independently selected from the Group Two Substituents; and
   (v) C1-C6 linear or branched heteroalkyl containing 1, 2, or 3 atoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group Two Substituents;
(n) 3- to 9-membered cycloheteroalkyl having 1, 2, or 3 heteroatoms independently selected from O, N, and S, optionally substituted with 1, 2, 3, or 4 substituents independently selected from
   (i) phenyl;
   (ii) halide;
   (iii) cyano;
   (iv) C1-C6 linear or branched alkyl, optionally substituted with
      (1) 1, 2, 3, 4, 5, 6, 7, 8, or 9 fluorine atoms; and/or
      (2) 1, 2, or 3 substituents independently selected from the Group Two Substituents; and
   (v) C1-C6 linear or branched heteroalkyl containing 1, 2, or 3 atoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group Two Substituents; and
(o) C3-C6 cycloalkyl, optionally substituted with 1, 2, 3, or 4 substituents independently selected from
   (i) phenyl;
   (ii) halide;
   (iii) cyano;
   (iv) C1-C6 linear or branched alkyl, optionally substituted with
      (1) 1, 2, 3, 4, 5, 6, 7, 8, or 9 fluorine atoms; and/or
      (2) 1, 2, or 3 substituents independently selected from the Group Two Substituents; and
   (v) C1-C6 linear or branched heteroalkyl containing 1, 2, or 3 atoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group Two Substituents.

“Group Two Substituents” is a group of substituents consisting of:
(a) —OH;
(b) —NH₂;
(c) ═O;
(d) ═S;
(e) ═NR7, where R7 is H or is C1-C3 linear or branched alkyl or C1-C3 linear or branched heteroalkyl comprising an O, N, or S atom;
(f) —C(O)OR4, wherein R4 is H or C1-C3 linear or branched alkyl;
(g) —C(O)NR5R6, wherein R5 and R6 independently are H or C1-C6 linear or branched alkyl;
(h) halide;
(i) cyano;
(j) trifluoromethyl;
(k) C1-C6 linear or branched alkoxyl;
(l) C1-C6 linear or branched alkylamino;
(m) C1-C6 linear or branched dialkylamino;
(n) 6- to 10-membered aromatic; and
(o) 5- to 10-membered heteroaromatic comprising 1, 2, 3, 4, 5, or 6 heteroatoms independently selected from O, N, and S.

The definitions above apply to the descriptions that follow. For example, the phrase “R4 is H or C1-C3 linear or branched alkyl” should be read as describing each of five sets of embodiments in which R₄ is H, R₄ is methyl, R₄ is ethyl, R₄ is propyl, and R₄ is isopropyl, respectively.

Therapeutic Agent Component(s)

A “therapeutic agent component” as used in this disclosure is a therapeutic moiety or portion of a therapeutic agent that is present in a conjugate molecule and covalently attached to a linker. A number of therapeutic agents can be used to provide a therapeutic agent component of a conjugate molecule.

Epoxides

In some embodiments, the therapeutic agent component is an epoxide. An example of how a cannabinoid could be released from a conjugate molecule upon binding of an epoxide to a target is shown below. The target's molecular structure is understood to contain nucleophilic groups such as NH, OH, and SH capable of reacting with the epoxide agent.

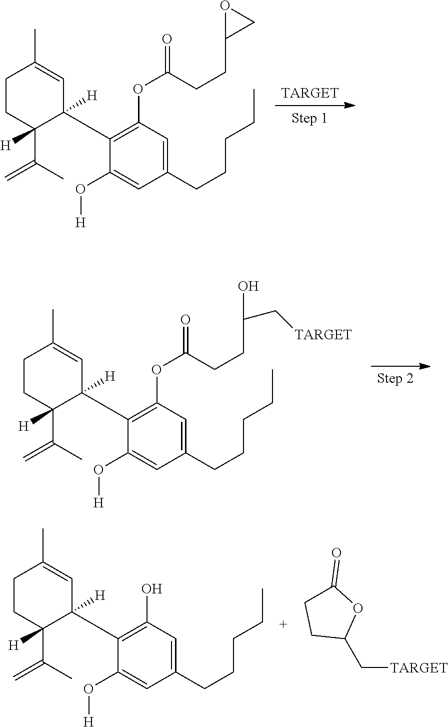

Epoxide components of a conjugate molecule have the following structure:

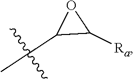

in which Rₐ is absent or is C1-C3 linear or branched alkyl or C1-C3 linear or branched heteroalkyl comprising a O, N, or S atom. Carfilzomib is an example of an epoxide.

Aziridines

In some embodiments, the therapeutic agent component is an aziridine. An example of how a cannabinoid could be released from a conjugate molecule upon binding of an aziridine to a target is shown below. The target's molecular structure is understood to contain nucleophilic groups such as NH, OH, and SH capable of reacting with the aziridine agent

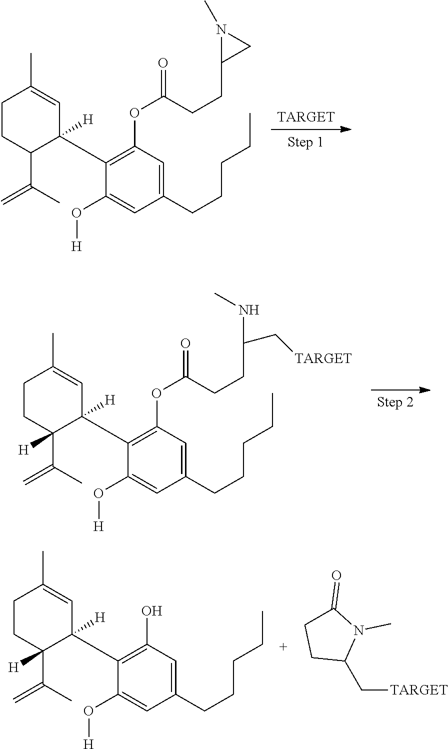

Aziridine components of a conjugate molecule have the following structure:

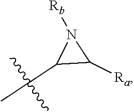

in which wherein Rₐ is absent or is C1-C3 linear or branched alkyl or C1-C3 linear or branched heteroalkyl comprising a O, N, or S atom; and R_{b} is R or —PS(NR_{c1}R_{c2}), wherein R_{c1} and R_{c2} independently are C1-C6 linear or branched alkyl or C1-C6 cycloalkyl, and wherein R is selected from the group consisting of:
(a) H;
(b) C1-C8 linear or branched alkyl, optionally substituted with
   (1) 1, 2, 3, 4, 5, 6, 7, 8, or 9 fluorine atoms; and/or
   (2) 1, 2, or 3 substituents independently selected from the Group One Substituents;
(c) C1-C8 linear or branched heteroalkyl containing 1, 2, or 3 heteroatoms independently selected from O, N, and S and optionally substituted with
   (1) 1, 2, 3, 4, 5, 6, 7, 8, or 9 fluorine atoms; and/or
   (2) 1, 2, or 3 substituents independently selected from the Group One Substituents;
(d) phenyl, optionally substituted with 1, 2, or 3 substituents independently selected from the group consisting of
   (1) C1-C6 linear or branched alkyl, optionally substituted with
      (i) 1, 2, 3, 4, 5, or 6 fluorine atoms; and/or
      (ii) 1 or 2 substituents independently selected from the Group Two Substituents; and
   (2) C1-C6 linear or branched heteroalkyl containing 1 or 2 heteroatoms independently selected from O, N, and S and optionally substituted with
      (i) 1, 2, 3, 4, 5, or 6 fluorine atoms; and/or
      (ii) 1 or 2 substituents independently selected from the Group One Substituents;
(e) a 6- to 10-membered aromatic, optionally substituted with 1, 2, 3, or 4 substituents independently selected from the group consisting of
   (1) phenyl;
   (2) halide;
   (3) cyano;
   (4) C1-C6 linear or branched alkyl, optionally substituted with
      (i) 1, 2, 3, 4, 5, 6, 7, 8, or 9 fluorine atoms; and/or
      (ii) 1, 2, or 3 substituents independently selected from the Group Two Substituents, and
   (5) C1-C6 linear or branched heteroalkyl containing 1, 2, or 3 atoms independently selected from O, N, and S and optionally substituted with
      (i) 1, 2, 3, 4, 5, 6, 7, 8, or 9 fluorine atoms; and/or
      (ii) 1, 2, or 3 substituents independently selected from the Group Two Substituents;
(f) 5- to 10-membered heteroaromatic comprising 1, 2, 3, 4, 5, or 6 heteroatoms independently selected from O, N, and S and optionally substituted with 1, 2, 3, or 4 substituents independently selected from
   (1) phenyl;
   (2) halide;
   (3) cyano;
   (4) trifluoromethyl;
   (5) C1-C6 linear or branched alkyl optionally substituted with
      (i) 1, 2, 3, 4, 5, 6, 7, 8, or 9 fluorine atoms; and/or
      (ii) 1, 2, or 3 substituents independently selected from the Group Two Substituents; and
   (6) C1-C6 linear or branched heteroalkyl containing 1, 2, or 3 atoms independently selected from O, N, and S and optionally substituted with
      (i) 1, 2, 3, 4, 5, 6, 7, 8, or 9 fluorine atoms; and/or
      (ii) 1, 2, or 3 substituents independently selected from the Group Two Substituents;
(g)

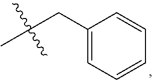 [SMILES: *Cc1ccccc1]

optionally substituted with 1, 2, or 3 substituents independently selected from the group consisting of:
   (1) C1-C6 linear or branched alkyl, optionally substituted with
      (i) 1, 2, 3, 4, 5, or 6 fluorine atoms; and/or
      (ii) 1, 2, or 3 substituents independently selected from the Group Two Substituents;
(h) 3- to 9-membered cycloheteroalkyl having 1, 2, or 3 heteroatoms independently selected from O, N, and S and optionally substituted with 1, 2, or 3 substituents independently selected from the group consisting of
   (1) C1-C6 linear or branched alkyl, optionally substituted with
      (i) 1, 2, 3, 4, 5, 6, 7, 8, or 9 fluorine atoms; and/or
      (ii) 1, 2, or 3 substituents independently selected from the Group Two Substituents,
   (2) C1-C6 linear or branched heteroalkyl, optionally substituted with
      (i) 1, 2, 3, 4, 5, 6, 7, 8, or 9 fluorine atoms and/or
      (ii) 1, 2, or 3 substituents independently selected from the Group Two Substituents,
   (3) phenyl, optionally substituted with 1, 2, or 3 substituents independently selected from the Group Two Substituents, and
   (4) 5- to 10-membered heteroaromatic, optionally substituted with 1, 2, or 3 substituents independently selected from the Group Two Substituents; and
(i) C3-C6 cycloalkyl, optionally substituted with 1, 2, or 3 substituents independently selected from:
   (1) C1-C6 linear or branched alkyl, optionally substituted with
      (i) 1, 2, 3, 4, 5, 6, 7, 8, or 9 fluorine atoms; and/or
      (ii) 1, 2, or 3 substituents independently selected from the Group Two Substituents,
   (2) C1-C6 linear or branched heteroalkyl, optionally substituted with
      (i) 1, 2, 3, 4, 5, 6, 7, 8, or 9 fluorine atoms; and/or
      (ii) 1, 2, or 3 substituents independently selected from the Group Two Substituents,
   (3) phenyl, optionally substituted with 1, 2, or 3 substituents independently selected from Group Two Substituents; and
   (4) 5- to 10-membered heteroaromatic, optionally substituted with 1, 2, or 3 substituents independently selected from the Group Two Substituents.

In some embodiments, R is selected from the group consisting of
(a) H;
(b) C1-C6 linear or branched alkyl, optionally substituted with
   (i) up to 9 fluorine atoms (i.e., 1, 2, 3, 4, 5, 6, 7, 8, or 9 fluorine atoms); and/or
   (ii) up to three substituents (i.e., 1, 2, or 3) selected from Group One Substituents;
(c) C1-C6 linear or branched heteroalkyl containing 1-3 heteroatoms (1, 2, or 3) independently selected from O, N, and S, optionally substituted with
   (i) up to 9 fluorine atoms; and/or
   (ii) up to three substituents selected from the Group One Substituents;
(d) phenyl, optionally substituted with 1-3 (i.e., 1, 2, or 3) substitutents independently selected from the group consisting of
   (i) C1-C6 linear or branched alkyl; and
   (ii) C1-C6 linear or branched heteroalkyl containing 1 or 2 heteroatoms independently selected from O, N, and S, optionally substituted with 1-6 fluorine atoms (i.e., 1, 2, 3, 4, 5, or 6 fluorine atoms) and/or 1 or 2 substituents selected from the Group One Substituents and halide; and
(e)

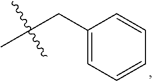 [SMILES: *Cc1ccccc1]

optionally substituted with 1-3 substituents independently selected from
   (i) C1-C6 linear or branched alkyl; and
   (ii) C1-C6 linear or branched heteroalkyl containing 1 or 2 heteroatoms independently selected from O, N, and S, optionally substituted with 1-6 fluorine atoms and/or 1 or 2 substituents selected from the Group One Substituents.

Sulfonates

In some embodiments, the therapeutic agent component is a sulfonate. Examples of how a cannabinoid could be released from a conjugate molecule upon binding of a sulfonate to a target are shown below. The target's molecular structure is understood to contain nucleophilic groups such as NH, OH, and SH capable of reacting with the sulfonate agent. While these examples utilize a NH₂ group such as from a lysine residue in both Steps 1 and 2, it is understood that the second step may use an entirely different nucleophilic group on the target to attack the link and release the cannabinoid.

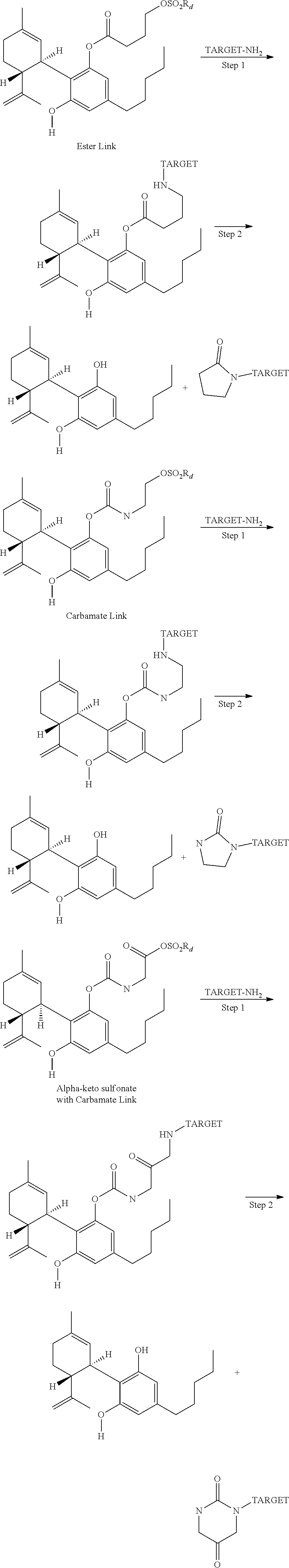

Sulfonate components of a conjugate molecule have the following structure:

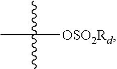

in which R_{d} is either (a) C1-C8 linear or branched alkyl, optionally substituted with (i) up to 9 fluorine atoms; and/or (ii) up to three substituents independently selected from the Group One Substituents; or (b) phenyl, optionally substituted with up to three substituents independently selected from the group consisting of C1-C6 linear or branched alkyl, optionally substituted with (i) up to 6 fluorine atoms and/or 1 or 2 substituents independently selected from the Group Two Substituents.

Halides

In some embodiments, the therapeutic agent component is a halide. Examples of how a cannabinoid could be released from a conjugate molecule upon binding of a halide to a target are shown below. The target's molecular structure is understood to contain nucleophilic groups such as NH, OH, and SH capable of reacting with the halide agent. While these examples utilize a NH₂ group such as from a lysine residue in both Steps 1 and 2, it is understood that the second step may use an entirely different nucleophilic group on the target to attack the link and release the cannabinoid.

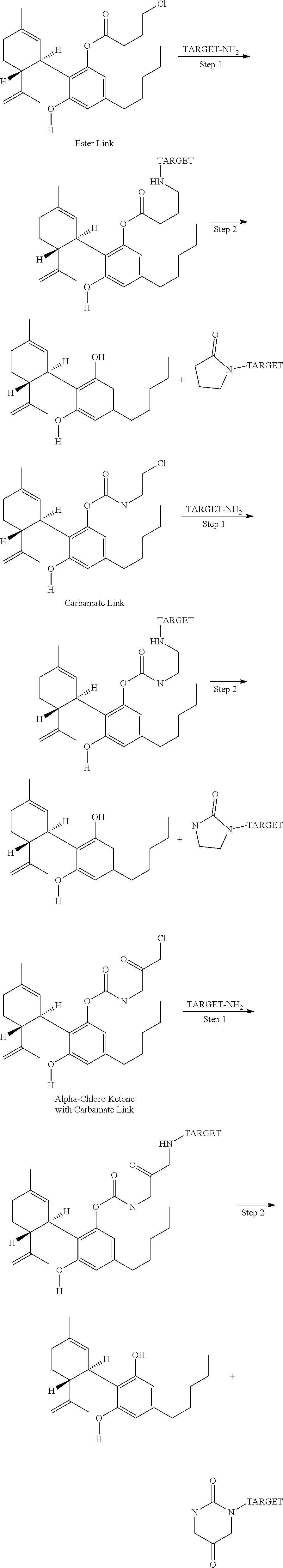

Halide components of a conjugate molecule have the structure

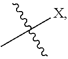

in which X is Cl, Br, or I.

Temozolomide and Temozolomide Analogs

In some embodiments, the therapeutic agent component is temozolomide or an analog of temozolomide, which is a DNA methylating/alkylating agent:

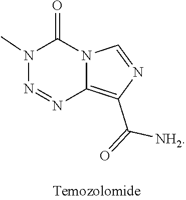

An example of how a cannabinoid may be released from a conjugate molecule upon binding of a temozolomide analog component to a target is shown below. The target's molecular structure is understood to contain nucleophilic groups such as NH, OH, and SH capable of reacting with the alkylating agent. While this example uses an NH₂ group such as from a guanine system in both Steps 1 and 2, it is understood that the second step may use an entirely different nucleophilic group on the target to attack the link and release the cannabinoid.

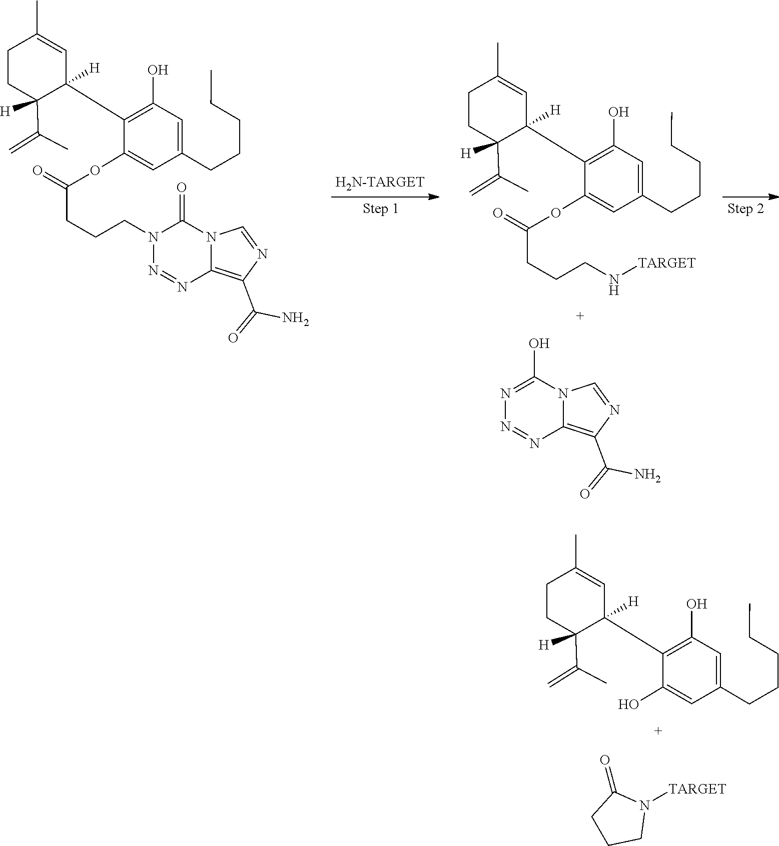

In some embodiments, temozolomide analog components of a conjugate molecule have the structure

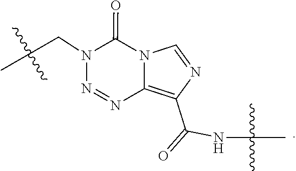 [SMILES: *Cn1nnc2c(C(=O)N*)ncn2c1=O]

In some embodiments, temozolomide analog components of a conjugate molecule have the structure

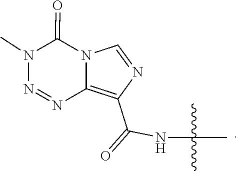 [SMILES: *NC(=O)c1ncn2c(=O)n(C)nnc12]

In some embodiments, temozolomide analog components of a conjugate molecule have the structure:

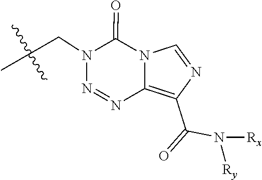

in which Rₓ and R_{y} independently are H or C1-C3 linear or branched alkyl. In some embodiments, Rₓ is H and R_{y} is H. In some embodiments, Rₓ is C1-C3 linear or branched alkyl and R_{y} is H. In some embodiments, both Rₓ and R_{y} are independently selected from C1-C3 linear or branched alkyl.

5-Fluorouracil and 5-Fluorouracil Analogs

In some embodiments, the therapeutic agent component is 5-fluorouracil (alone or as part of a 5-fluorouracil-containing product, such as VERRUCA HERMAL (5-fluorouracil, salicylic acid) or an analog of 5-fluorouracil:

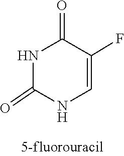

Examples of how a cannabinoid can be released from a conjugate molecule upon binding of a 5-fluorouracil analog component to a target are shown below. The target's molecular structure is understood to contain nucleophilic groups such as NH, OH, and SH capable of reacting at the 6-position of the uracil system, or the 6-position of its FdUMP metabolite. It is understood that the nucleophilic group attaching to the 6-position may be different from the nucleophilic group that reacts with the release the cannabinoid in Step 2.

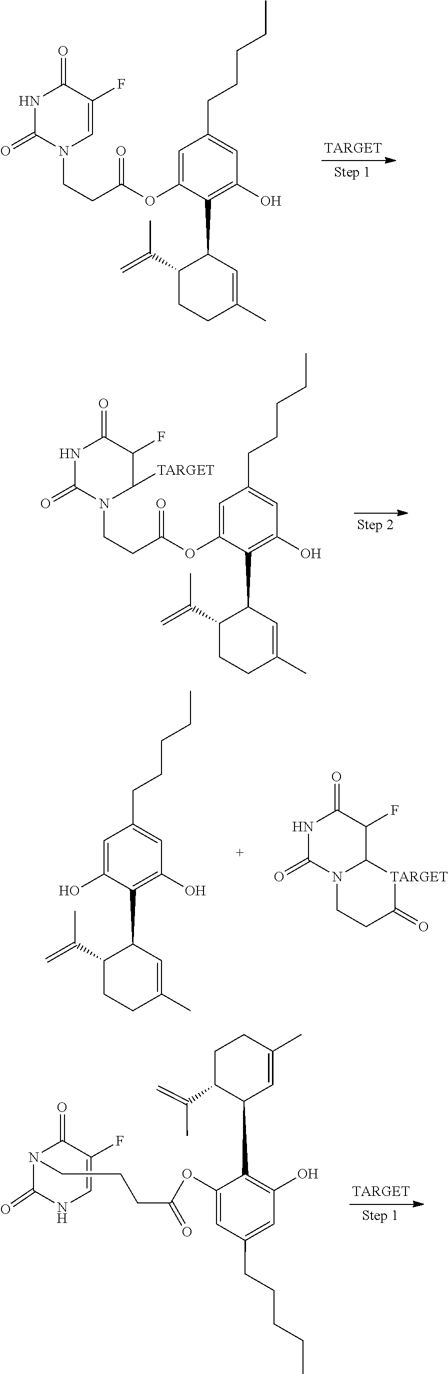

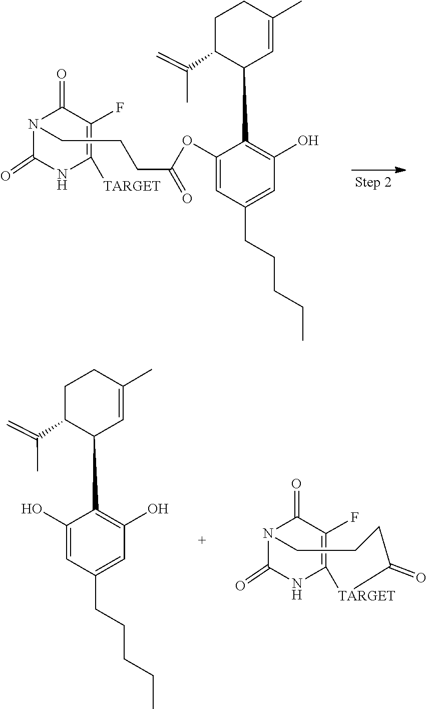

In some embodiments, the therapeutic agent component is

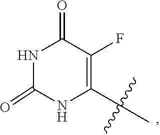 [SMILES: *c1[nH]c(=O)[nH]c(=O)c1F]

where [symbol] marks the bond covalently attaching the therapeutic agent component to the linker. In some embodiments, the therapeutic agent component is

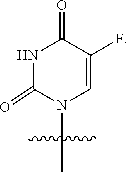 [SMILES: *n1cc(F)c(=O)[nH]c1=O]

In some embodiments, the therapeutic agent component is

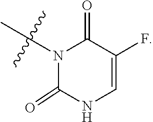 [SMILES: *n1c(=O)[nH]cc(F)c1=O]

In some embodiments, the therapeutic agent component is

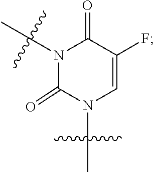 [SMILES: *n1cc(F)c(=O)n(*)c1=O]

in these embodiments, two cannabinoid components can be covalently attached via linkers to the therapeutic agent component. The two cannabinoid components can be the same or can be different; and, independently, the two linkers can be the same or different.

In some embodiments, the therapeutic agent component is

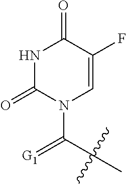

where G₁ is O, S, or NR. In some embodiments, the therapeutic agent component is

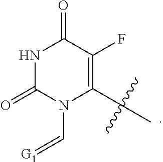

In some embodiments, the therapeutic agent component is

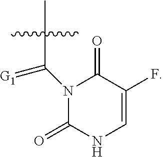

In some embodiments, the therapeutic agent component is

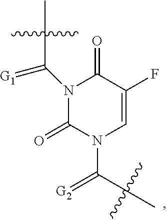

in which G₁ and G₂ independently are selected from O, S, and NR; in these embodiments, two cannabinoid components can be covalently attached via linkers to the therapeutic agent component. The two cannabinoid components can be the same or can be different; and, independently, the two linkers can be the same or different.

In some embodiments, the therapeutic agent component is diclofenac or an analog of diclofenac:

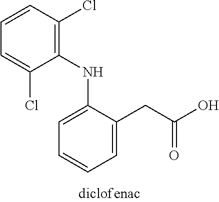 [SMILES: O=C(O)Cc1ccccc1Nc1c(Cl)cccc1Cl]

In some embodiments, a diclofenac component has the structure

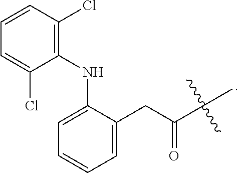 [SMILES: *C(=O)Cc1ccccc1Nc1c(Cl)cccc1Cl]

In some embodiments, a diclofenac component has the structure

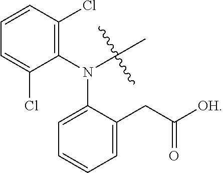 [SMILES: *N(c1ccccc1CC(=O)O)c1c(Cl)cccc1Cl]

In some embodiments, a diclofenac component has the structure

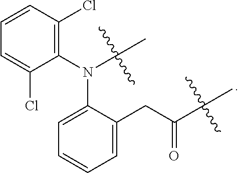 [SMILES: *C(=O)Cc1ccccc1N(*)c1c(Cl)cccc1Cl]

Conjugates comprising a diclofenac component can be administered alone or, for example, as part of a diclofenac-containing product, such as MOBIZOX® (diclofenac, paracetamol, and chlozoxazone), SOLARAZE® (diclofenac sodium), VOLTAREN® (diclofenac sodium), VOLITRA® (benzyl alcohol, capsaicin, diclofenac diethylamine, linseed oil, menthol, methyl salicylate), VOLITRA® MR (diclofenac, thiocolchicoside), VOLITRA® PLUS (diclofenac dethylamine, linseed oil, methyl salicylate, menthol, eucalyptus oil), VOLITRA® S (diclofenac sodium ip, serratiopeptidase), FLEXURA® D (diclofenac potassium bp, metaxalone), MOBISWIFT® D (diclofenac, methoxolone), THIOACT® D (thiocochicoside, diclofenac sodium ip).

In some embodiments, the therapeutic agent component is celecoxib (e.g., CELEBREX®) or an analog of celecoxib:

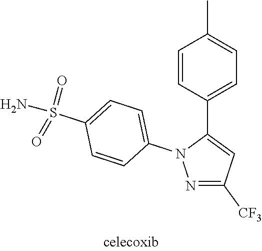 [SMILES: Cc1ccc(-c2cc(C(F)(F)F)nn2-c2ccc(S(N)(=O)=O)cc2)cc1]

In some embodiments, a celecoxib component has the structure

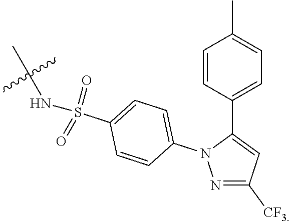 [SMILES: *NS(=O)(=O)c1ccc(-n2nc(C(F)(F)F)cc2-c2ccc(C)cc2)cc1]

In some embodiments, the therapeutic agent component is gemcitabine (e.g., GEMZAR®) or an analog of gemcitabine:

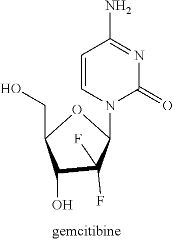

In some embodiments, a gemcitabine component has the structure

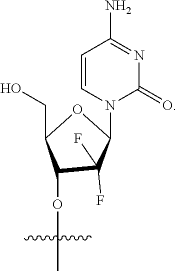

In some embodiments, a gemcitabine component has the structure

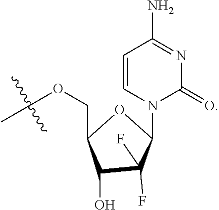

In some embodiments, a gemcitabine component has the structure

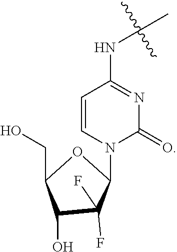

In some embodiments, a gemcitabine component has the structure

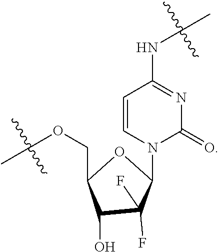

In some embodiments, a gemcitabine component has the structure

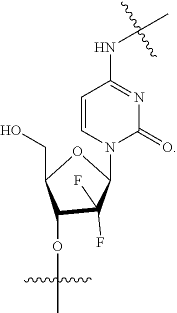

In some embodiments, a gemcitabine component has the structure

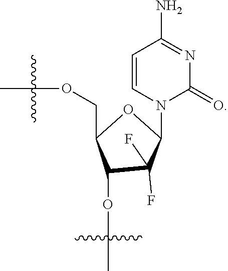

In some embodiments, a gemcitabine component has the structure

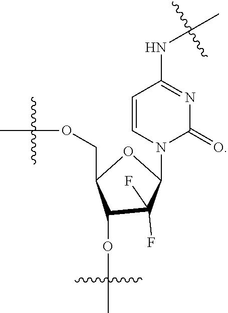

In some embodiments, the therapeutic agent component is or emtricitabine (e.g., DESCOVY®, BIKTARVY®, EMTRIVA®) or an analog of emtricitabine:

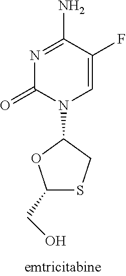 [SMILES: Nc1nc(=O)n([C@@H]2CS[C@H](CO)O2)cc1F]

In some embodiments, an emtricitabine component has the structure

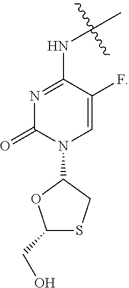

In some embodiments, an emtricitabine component has the structure

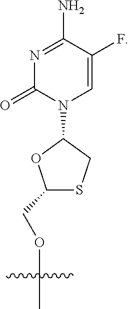 [SMILES: *OC[C@@H]1O[C@H](n2cc(F)c(N)nc2=O)CS1]

In some embodiments, an emtricitabine component has the structure

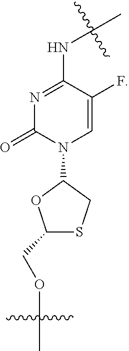

In some embodiments, the therapeutic agent component is entecavir (e.g., BARACLUDE®) or an analog of entecavir:

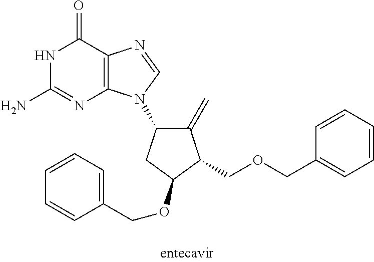 [SMILES: C=C1[C@H](COCc2ccccc2)[C@@H](OCc2ccccc2)C[C@@H]1n1cnc2c(=O)[nH]c(N)nc21]

In some embodiments an entecavir component has the structure:

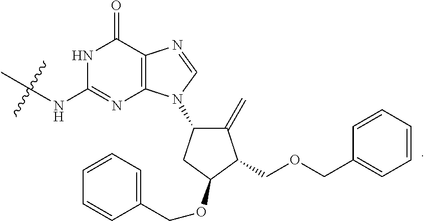 [SMILES: *Nc1nc2c(ncn2[C@H]2C[C@H](OCc3ccccc3)[C@@H](COCc3ccccc3)C2=C)c(=O)[nH]1]

In some embodiments, the therapeutic agent component is axitinib (e.g., INLYTA®) or an analog of axitinib:

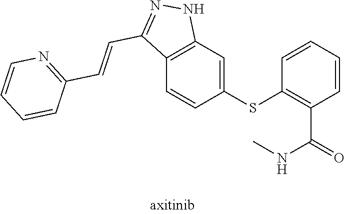 [SMILES: CNC(=O)c1ccccc1Sc1ccc2c(/C=C/c3ccccn3)n[nH]c2c1]

In some embodiments, an axitinib component has the structure

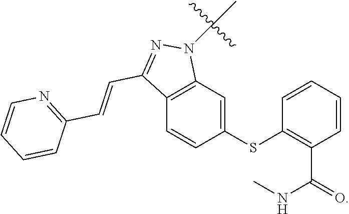 [SMILES: *n1nc(/C=C/c2ccccn2)c2ccc(Sc3ccccc3C(=O)NC)cc21]

In some embodiments, the therapeutic agent component is batimastat or an analog of batimastat:

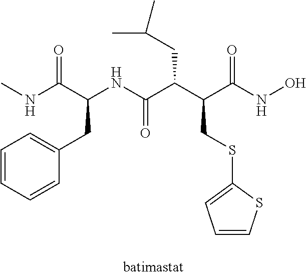 [SMILES: CNC(=O)[C@H](Cc1ccccc1)NC(=O)[C@H](CC(C)C)[C@H](CSc1cccs1)C(=O)NO]

In some embodiments, a batimastat component has the structure

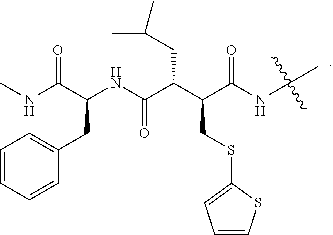 [SMILES: *NC(=O)[C@@H](CSc1cccs1)[C@@H](CC(C)C)C(=O)N[C@@H](Cc1ccccc1)C(=O)NC]

In some embodiments, the therapeutic agent component is bosutinib (e.g., BOSULIF®) or an analog of bosutinib:

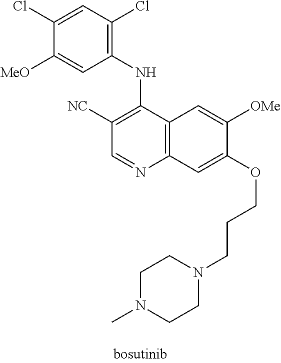 [SMILES: COc1cc(Nc2c(C#N)cnc3cc(OCCCN4CCN(C)CC4)c(OC)cc23)c(Cl)cc1Cl]

In some embodiments, a bosutinib component has the structure

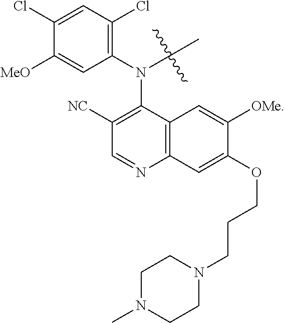 [SMILES: *N(c1cc(OC)c(Cl)cc1Cl)c1c(C#N)cnc2cc(OCCCN3CCN(C)CC3)c(OC)cc12]

In some embodiments, the therapeutic agent component is crizotinib (e.g., XALKORI®) or an analog of crizotinib:

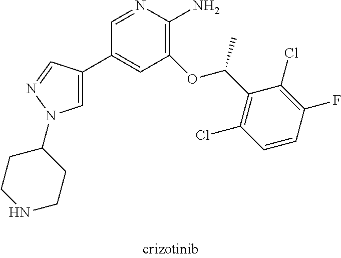 [SMILES: C[C@@H](Oc1cc(-c2cnn(C3CCNCC3)c2)cnc1N)c1c(Cl)ccc(F)c1Cl]

In some embodiments, a crizotinib component has the structure

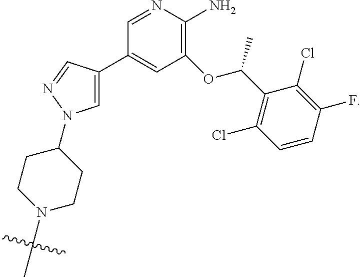 [SMILES: *N1CCC(n2cc(-c3cnc(N)c(O[C@H](C)c4c(Cl)ccc(F)c4Cl)c3)cn2)CC1]

In some embodiments, a crizotinib component has the structure

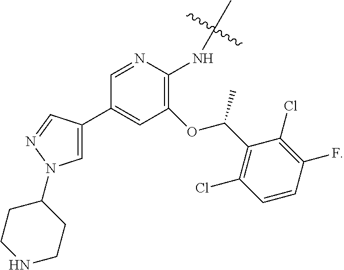 [SMILES: *Nc1ncc(-c2cnn(C3CCNCC3)c2)cc1O[C@H](C)c1c(Cl)ccc(F)c1Cl]

In some embodiments, a crizotinib component has the structure

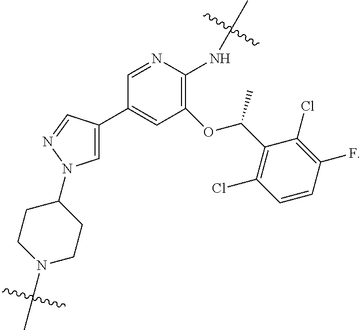 [SMILES: *Nc1ncc(-c2cnn(C3CCN(*)CC3)c2)cc1O[C@H](C)c1c(Cl)ccc(F)c1Cl]

In some embodiments, the therapeutic agent component is erlotinib (e.g., TARCEVA®) or an analog of erlotinib:

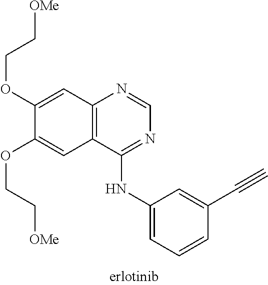 [SMILES: C#Cc1cccc(Nc2ncnc3cc(OCCOC)c(OCCOC)cc23)c1]

In some embodiments, an erlotinib component has the structure

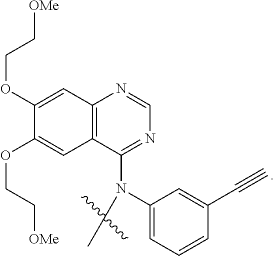 [SMILES: *N(c1cccc(C#C)c1)c1ncnc2cc(OCCOC)c(OCCOC)cc12]

In some embodiments, the therapeutic agent component is gefitinib (e.g., IRESSA®) or an analog of gefitinib:

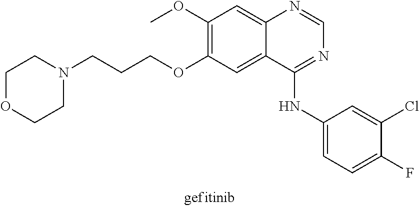 [SMILES: COc1cc2ncnc(Nc3ccc(F)c(Cl)c3)c2cc1OCCCN1CCOCC1]

In some embodiments, a gefitinib component has the structure

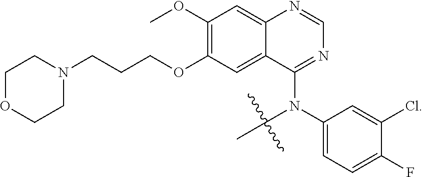 [SMILES: *N(c1ccc(F)c(Cl)c1)c1ncnc2cc(OC)c(OCCCN3CCOCC3)cc12]

In some embodiments, the therapeutic agent component is everolimus (e.g., ZORTRESS®, AFINITOR DISPERZ®, AFINITOR®) or an analog of everolimus:

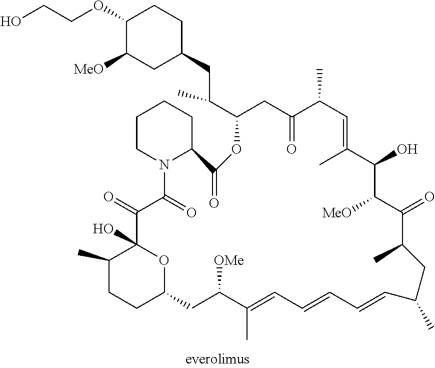 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, an everolimus component has the structure

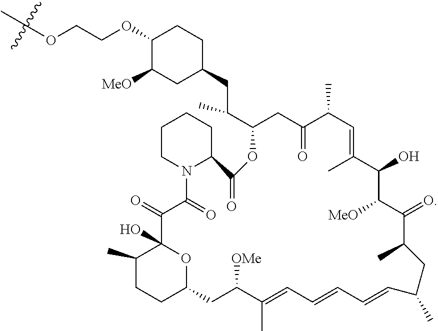 [SMILES: *OCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

In some embodiments, an everolimus component has the structure

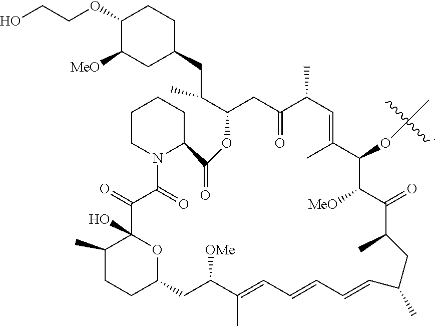 [SMILES: *O[C@@H]1/C(C)=C/[C@@H](C)C(=O)C[C@@H]([C@H](C)C[C@@H]2CC[C@@H](OCCO)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@@H]1OC]

In some embodiments, an everolimus component has the structure

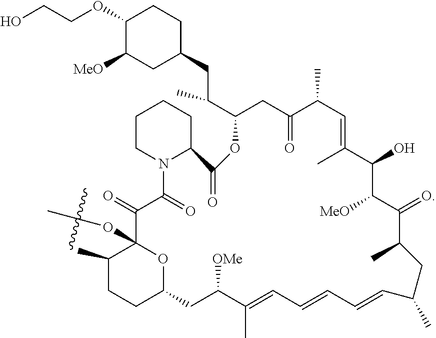 [SMILES: *O[C@@]12O[C@@H](CC[C@H]1C)C[C@H](OC)/C(C)=C/C=C/C=C/[C@@H](C)C[C@@H](C)C(=O)[C@H](OC)[C@H](O)/C(C)=C/[C@@H](C)C(=O)C[C@@H]([C@H](C)C[C@@H]1CC[C@@H](OCCO)[C@H](OC)C1)OC(=O)[C@@H]1CCCCN1C(=O)C2=O]

In some embodiments, an everolimus component has the structure

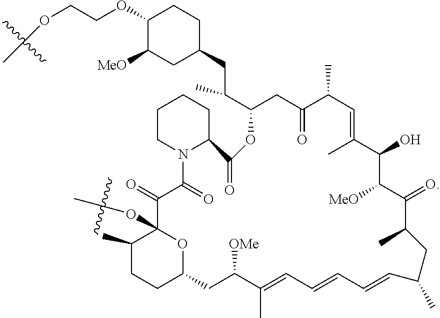 [SMILES: *OCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O*)(O3)C(=O)C(=O)N3CCCC[C@H]3C(=O)O2)C[C@H]1OC]

In some embodiments, an everolimus component has the structure

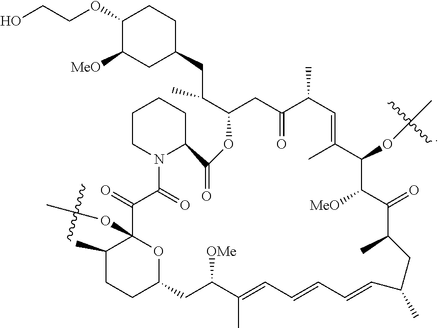 [SMILES: *O[C@@H]1/C(C)=C/[C@@H](C)C(=O)C[C@@H]([C@H](C)C[C@@H]2CC[C@@H](OCCO)[C@H](OC)C2)OC(=O)[C@@H]2CCCCN2C(=O)C(=O)[C@]2(O*)O[C@@H](CC[C@H]2C)C[C@H](OC)/C(C)=C/C=C/C=C/[C@@H](C)C[C@@H](C)C(=O)[C@@H]1OC]

In some embodiments, an everolimus component has the structure

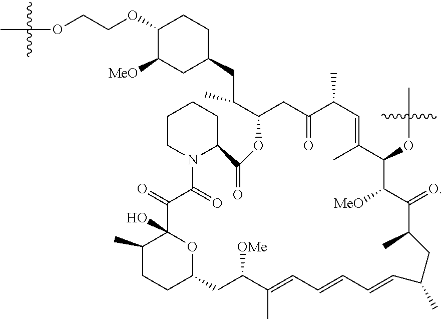 [SMILES: *OCCO[C@@H]1CC[C@@H](C[C@@H](C)[C@@H]2CC(=O)[C@H](C)/C=C(\C)[C@@H](O*)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

In some embodiments, an everolimus component has the structure

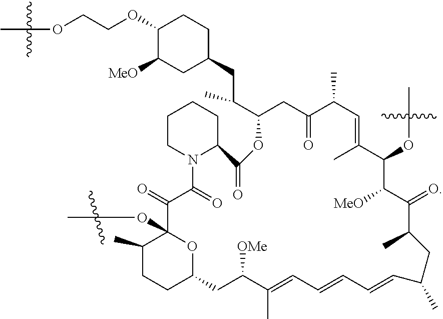 [SMILES: *OCCO[C@@H]1CC[C@@H](C[C@@H](C)[C@@H]2CC(=O)[C@H](C)/C=C(\C)[C@@H](O*)[C@@H](OC)C(=O)[C@H](C)C[C@H](C)/C=C/C=C/C=C(\C)[C@@H](OC)C[C@@H]3CC[C@@H](C)[C@@](O*)(O3)C(=O)C(=O)N3CCCC[C@H]3C(=O)O2)C[C@H]1OC]

In some embodiments, the therapeutic agent component is temsirolimus (e.g., TORISEL®) or an analog of temsirolimus:

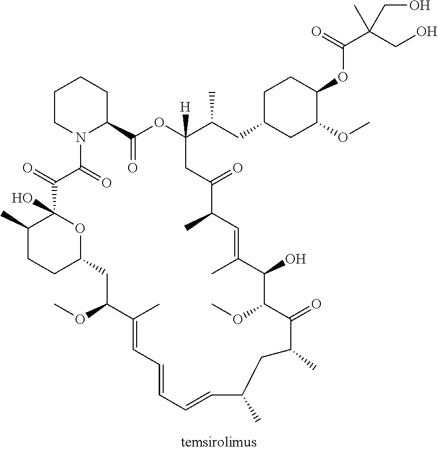 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, a temsirolimus component has one of the following structures, in which each arrow indicates a point where a linker as described below can be attached.

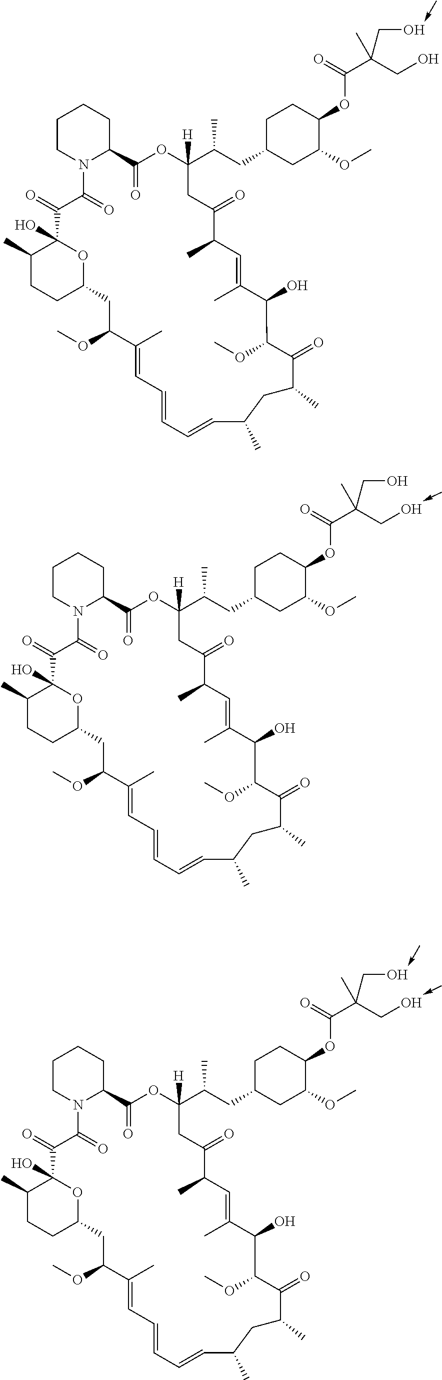

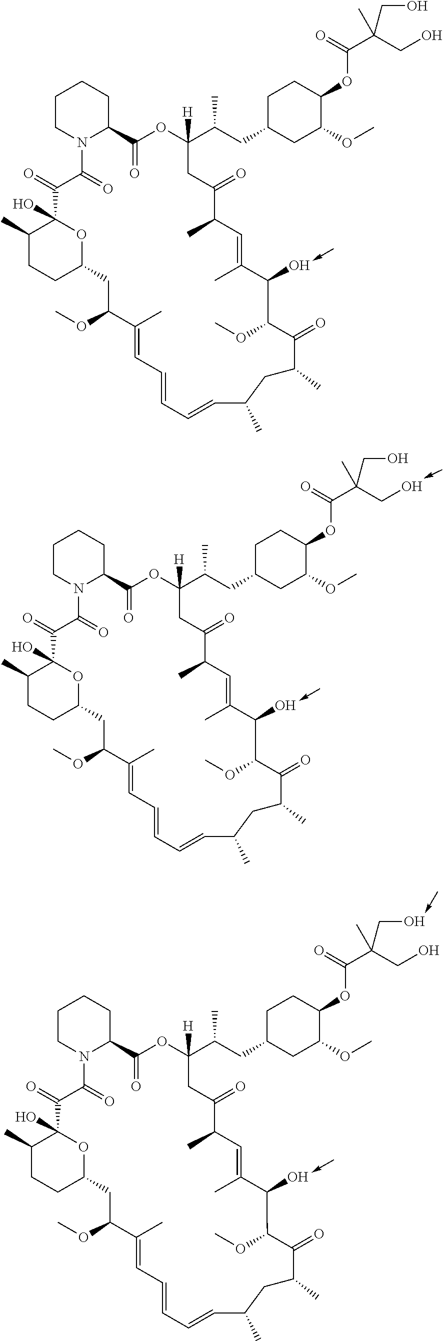

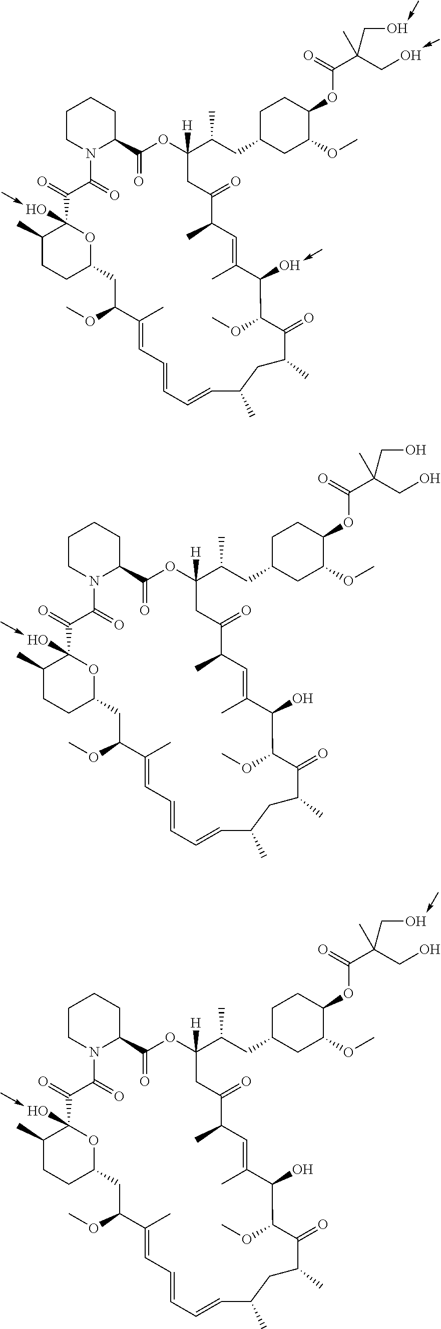

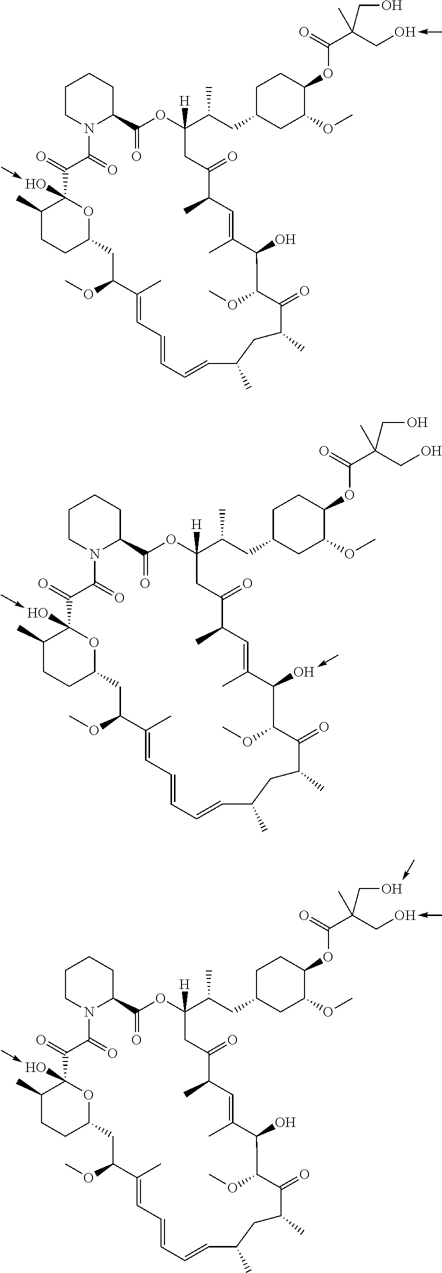

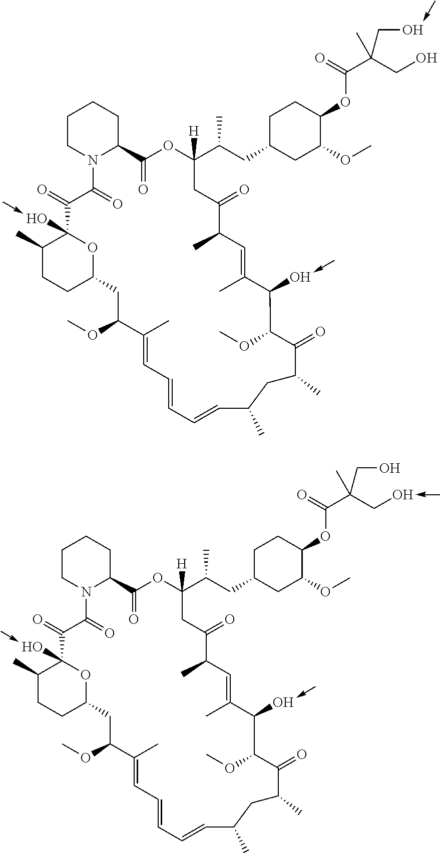

In some embodiments, the therapeutic agent component is ganetespib or an analog of ganetespib:

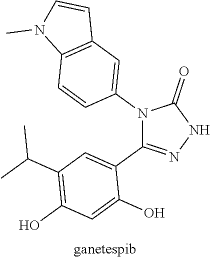 [SMILES: CC(C)c1cc(-c2n[nH]c(=O)n2-c2ccc3c(ccn3C)c2)c(O)cc1O]

In some embodiments, a ganetespib component has the structure

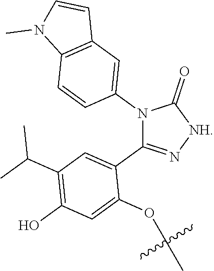 [SMILES: *Oc1cc(O)c(C(C)C)cc1-c1n[nH]c(=O)n1-c1ccc2c(ccn2C)c1]

In some embodiments, a ganetespib component has the structure

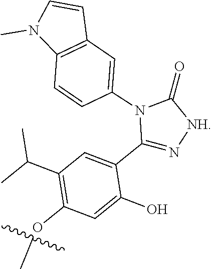

In some embodiments, a ganetespib component has the structure

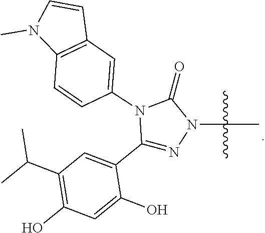 [SMILES: *n1nc(-c2cc(C(C)C)c(O)cc2O)n(-c2ccc3c(ccn3C)c2)c1=O]

In some embodiments, a ganetespib component has the structure

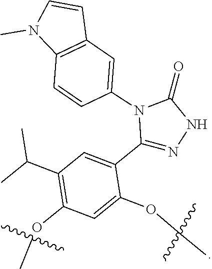

In some embodiments, a ganetespib component has the structure

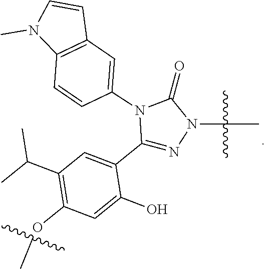

In some embodiments, a ganetespib component has the structure

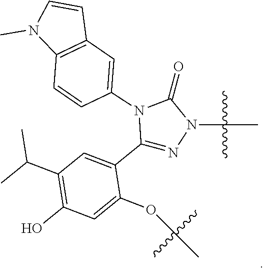 [SMILES: *Oc1cc(O)c(C(C)C)cc1-c1nn(*)c(=O)n1-c1ccc2c(ccn2C)c1]

In some embodiments, a ganetespib component has the structure

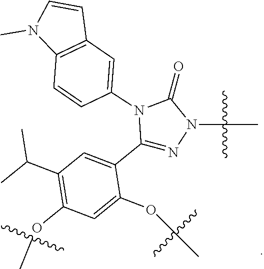

In some embodiments, the therapeutic agent component is glasdegib (e.g., GLASDEGIB®) or an analog of glasdegib:

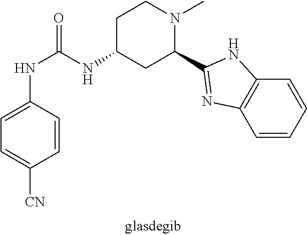 [SMILES: CN1CC[C@@H](NC(=O)Nc2ccc(C#N)cc2)C[C@@H]1c1nc2ccccc2[nH]1]

In some embodiments, a glasdegib component has the structure

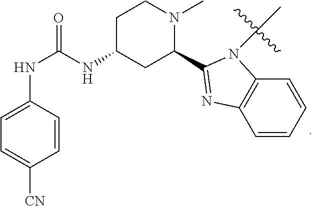 [SMILES: *n1c([C@H]2C[C@H](NC(=O)Nc3ccc(C#N)cc3)CCN2C)nc2ccccc21]

In some embodiments, the therapeutic agent component is imatinib (e.g., GLEEVEC®) or an analog of imatinib:

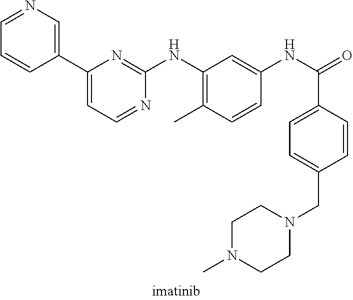 [SMILES: Cc1ccc(NC(=O)c2ccc(CN3CCN(C)CC3)cc2)cc1Nc1nccc(-c2cccnc2)n1]

In some embodiments, an imatinib component has the structure

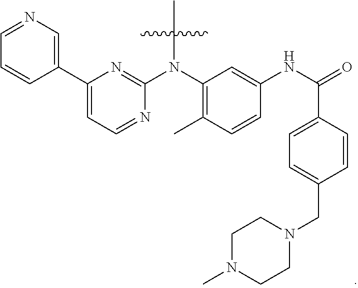 [SMILES: *N(c1nccc(-c2cccnc2)n1)c1cc(NC(=O)c2ccc(CN3CCN(C)CC3)cc2)ccc1C]

In some embodiments, an imatinib component has the structure

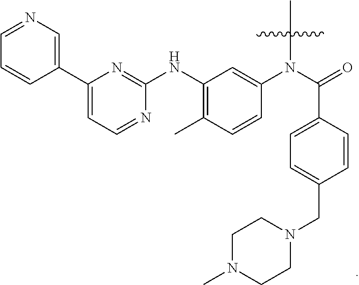 [SMILES: *N(C(=O)c1ccc(CN2CCN(C)CC2)cc1)c1ccc(C)c(Nc2nccc(-c3cccnc3)n2)c1]

In some embodiments, an imatinib component has the structure

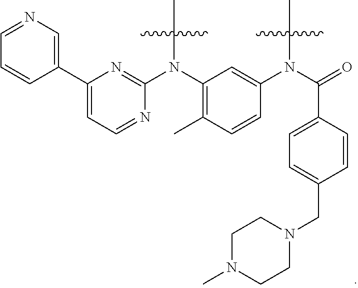 [SMILES: *N(C(=O)c1ccc(CN2CCN(C)CC2)cc1)c1ccc(C)c(N(*)c2nccc(-c3cccnc3)n2)c1]

In some embodiments, the therapeutic agent component is lapatinib (e.g., TYKERB®) or an analog of lapatinib:

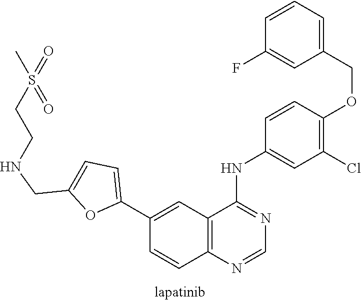 [SMILES: CS(=O)(=O)CCNCc1ccc(-c2ccc3ncnc(Nc4ccc(OCc5cccc(F)c5)c(Cl)c4)c3c2)o1]

In some embodiments a lapatinib component has the structure

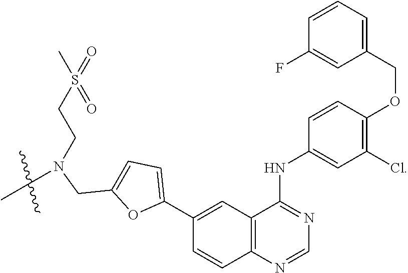 [SMILES: *N(CCS(C)(=O)=O)Cc1ccc(-c2ccc3ncnc(Nc4ccc(OCc5cccc(F)c5)c(Cl)c4)c3c2)o1]

In some embodiments a lapatinib component has the structure

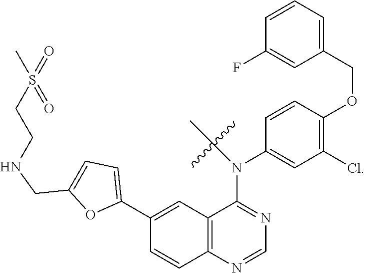 [SMILES: *N(c1ccc(OCc2cccc(F)c2)c(Cl)c1)c1ncnc2ccc(-c3ccc(CNCCS(C)(=O)=O)o3)cc12]

In some embodiments a lapatinib component has the structure

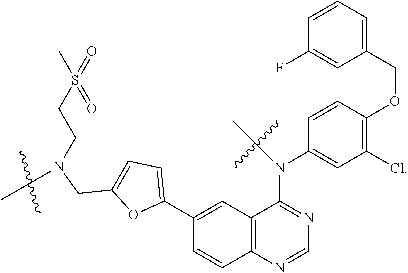 [SMILES: *N(CCS(C)(=O)=O)Cc1ccc(-c2ccc3ncnc(N(*)c4ccc(OCc5cccc(F)c5)c(Cl)c4)c3c2)o1]

In some embodiments, the therapeutic agent component is navitoclax or an analog of navitoclax:

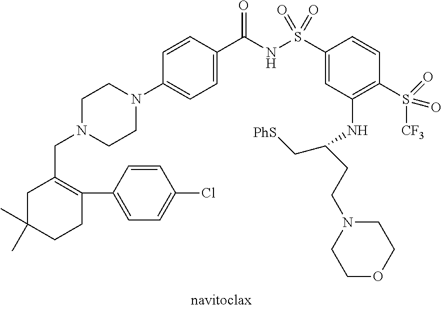

In some embodiments, a navitoclax component has the structure

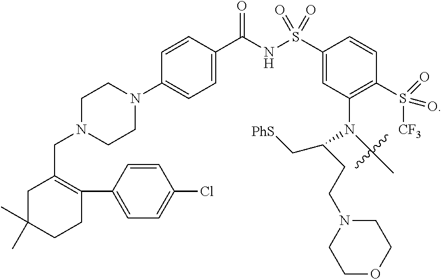

In some embodiments, a navitoclax component has the structure

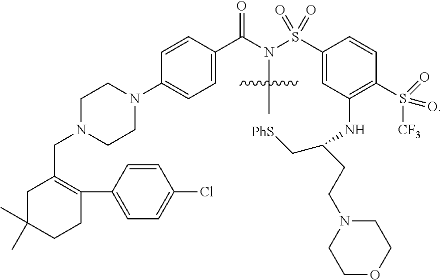

In some embodiments, a navitoclax component has the structure

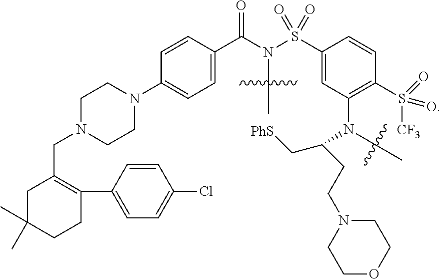

In some embodiments, the therapeutic agent component is nilotinib (e.g., TASIGNA®) or an analog of nilotinib:

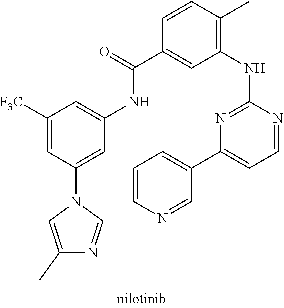 [SMILES: Cc1cn(-c2cc(NC(=O)c3ccc(C)c(Nc4nccc(-c5cccnc5)n4)c3)cc(C(F)(F)F)c2)cn1]

In some embodiments, a nilotinib component has the structure

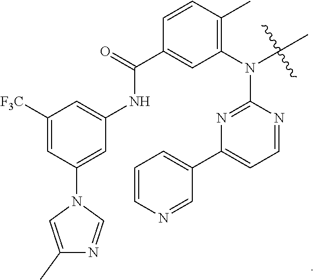 [SMILES: *N(c1nccc(-c2cccnc2)n1)c1cc(C(=O)Nc2cc(-n3cnc(C)c3)cc(C(F)(F)F)c2)ccc1C]

In some embodiments, a nilotinib component has the structure

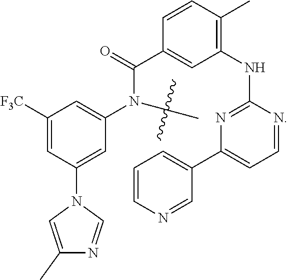 [SMILES: *N(C(=O)c1ccc(C)c(Nc2nccc(-c3cccnc3)n2)c1)c1cc(-n2cnc(C)c2)cc(C(F)(F)F)c1]

In some embodiments, a nilotinib component has the structure

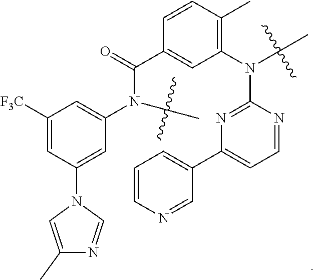 [SMILES: *N(C(=O)c1ccc(C)c(N(*)c2nccc(-c3cccnc3)n2)c1)c1cc(-n2cnc(C)c2)cc(C(F)(F)F)c1]

In some embodiments, the therapeutic agent component is pazopanib (e.g., OPDIVO®, VOTRIENT®) or an analog of pazopanib:

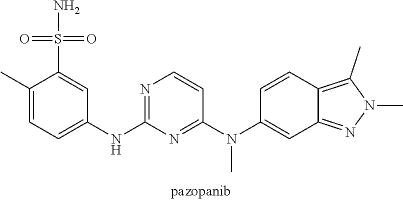

In some embodiments, a pazopanib component has the structure

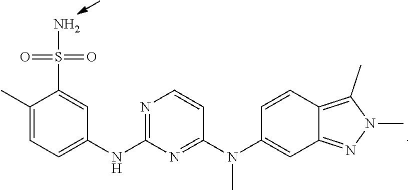

in some embodiments, a pazopanib component has the structure

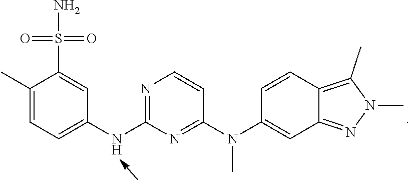

In some embodiments, a pazopanib component has the structure

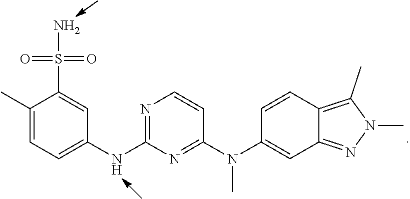

In some embodiments, the therapeutic agent component is luminespib or an analog of luminespib:

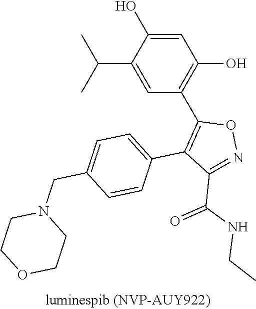 [SMILES: CCNC(=O)c1noc(-c2cc(C(C)C)c(O)cc2O)c1-c1ccc(CN2CCOCC2)cc1]

In some embodiments, a luminespib component has the structure

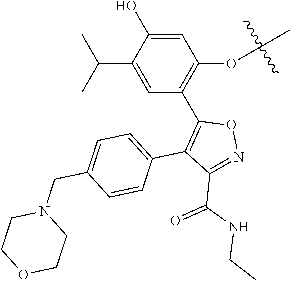 [SMILES: *Oc1cc(O)c(C(C)C)cc1-c1onc(C(=O)NCC)c1-c1ccc(CN2CCOCC2)cc1]

In some embodiments, a luminespib component has the structure

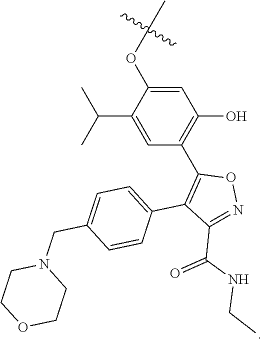 [SMILES: *Oc1cc(O)c(-c2onc(C(=O)NCC)c2-c2ccc(CN3CCOCC3)cc2)cc1C(C)C]

In some embodiments, a luminespib component has the structure

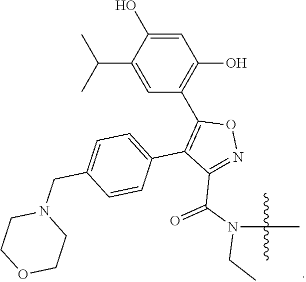 [SMILES: *N(CC)C(=O)c1noc(-c2cc(C(C)C)c(O)cc2O)c1-c1ccc(CN2CCOCC2)cc1]

In some embodiments, a luminespib component has the structure

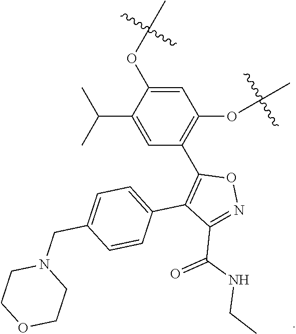 [SMILES: *Oc1cc(O*)c(C(C)C)cc1-c1onc(C(=O)NCC)c1-c1ccc(CN2CCOCC2)cc1]

In some embodiments, a luminespib component has the structure

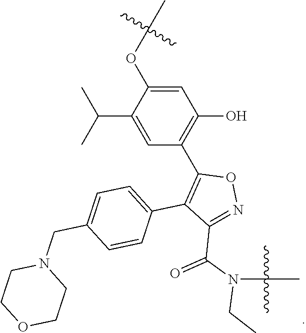 [SMILES: *Oc1cc(O)c(-c2onc(C(=O)N(*)CC)c2-c2ccc(CN3CCOCC3)cc2)cc1C(C)C]

In some embodiments, a luminespib component has the structure

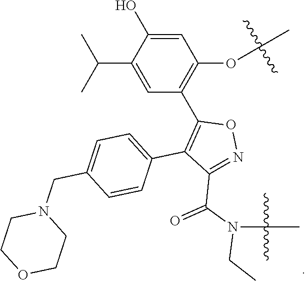 [SMILES: *Oc1cc(O)c(C(C)C)cc1-c1onc(C(=O)N(*)CC)c1-c1ccc(CN2CCOCC2)cc1]

In some embodiments, a luminespib component has the structure

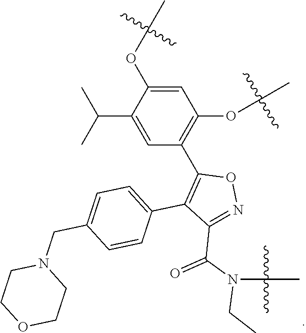 [SMILES: *Oc1cc(O*)c(C(C)C)cc1-c1onc(C(=O)N(*)CC)c1-c1ccc(CN2CCOCC2)cc1]

In some embodiments, the therapeutic agent component is obatoclax or an analog of obatoclax:

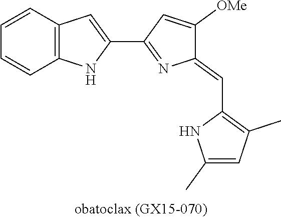 [SMILES: COC1=CC(c2cc3ccccc3[nH]2)=N/C1=C\c1[nH]c(C)cc1C]

In some embodiments, an obaoclax component has the structure

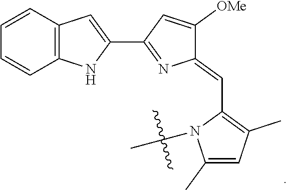 [SMILES: *n1c(C)cc(C)c1/C=C1\N=C(c2cc3ccccc3[nH]2)C=C1OC]

In some embodiments, an obatoclax component has the structure

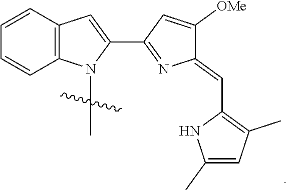 [SMILES: *n1c(C2=N/C(=C\c3[nH]c(C)cc3C)C(OC)=C2)cc2ccccc21]

In some embodiments, an obatoclax component has the structure

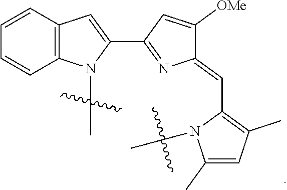 [SMILES: *n1c(C)cc(C)c1/C=C1\N=C(c2cc3ccccc3n2*)C=C1OC]

In some embodiments, the therapeutic agent component is ruxolitinib (e.g., JAKAFI®) or an analog of ruxolitinib:

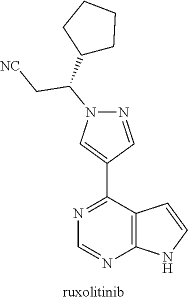 [SMILES: N#CC[C@H](C1CCCC1)n1cc(-c2ncnc3[nH]ccc23)cn1]

In some embodiments, a ruxolitinib component has the structure

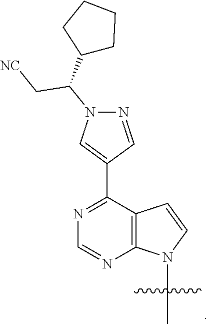 [SMILES: *n1ccc2c(-c3cnn([C@H](CC#N)C4CCCC4)c3)ncnc21]

In some embodiments, the therapeutic agent component is saridegib (e.g., ODOMZO®) or an analog of saridegib:

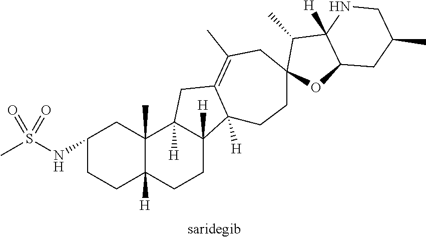

In some embodiments, a saridegib component has the structure

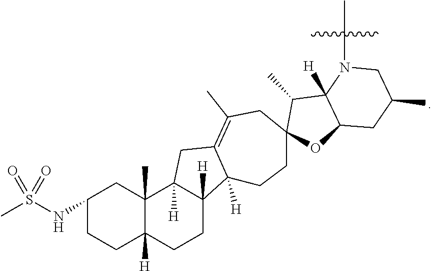

In some embodiments, a saridegib component has the structure

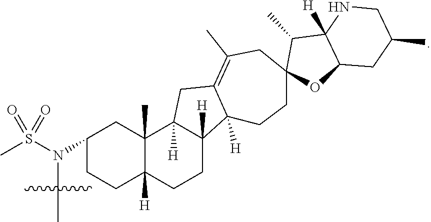

In some embodiments, a saridegib component has the structure

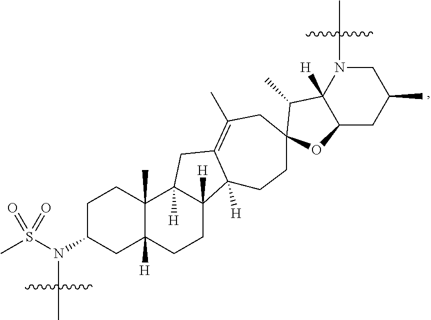

In some embodiments, the therapeutic agent component is sunitiib (e.g., SUTENT®) or an analog of sunitinib:

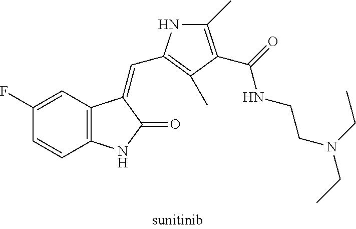 [SMILES: CCN(CC)CCNC(=O)c1c(C)[nH]c(/C=C2\C(=O)Nc3ccc(F)cc32)c1C]

In some embodiments, a sunitinib component has the structure:

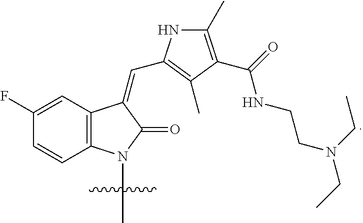 [SMILES: *N1C(=O)/C(=C\c2[nH]c(C)c(C(=O)NCCN(CC)CC)c2C)c2cc(F)ccc21]

In some embodiments, a sunitinib component has the structure

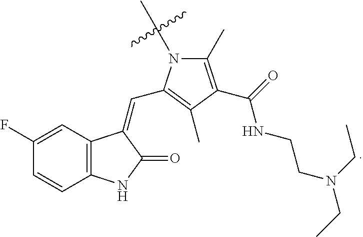 [SMILES: *n1c(C)c(C(=O)NCCN(CC)CC)c(C)c1/C=C1\C(=O)Nc2ccc(F)cc21]

In some embodiments, a sunitinib component has the structure

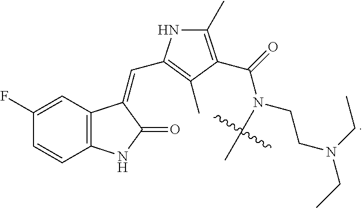 [SMILES: *N(CCN(CC)CC)C(=O)c1c(C)[nH]c(/C=C2\C(=O)Nc3ccc(F)cc32)c1C]

In some embodiments, a sunitinib component has the structure

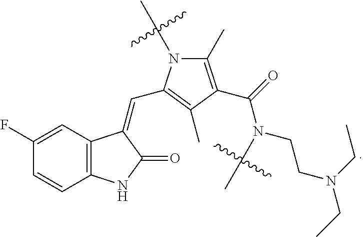 [SMILES: *N(CCN(CC)CC)C(=O)c1c(C)c(/C=C2\C(=O)Nc3ccc(F)cc32)n(*)c1C]

In some embodiments, a sunitinib component has the structure

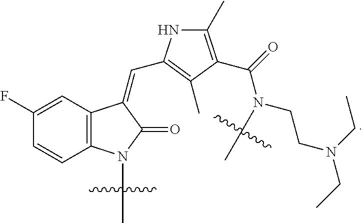 [SMILES: *N(CCN(CC)CC)C(=O)c1c(C)[nH]c(/C=C2\C(=O)N(*)c3ccc(F)cc32)c1C]

In some embodiments, a sunitinib component has the structure

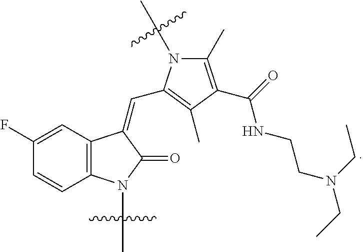 [SMILES: *N1C(=O)/C(=C\c2c(C)c(C(=O)NCCN(CC)CC)c(C)n2*)c2cc(F)ccc21]

In some embodiments, a sunitinib component has the structure

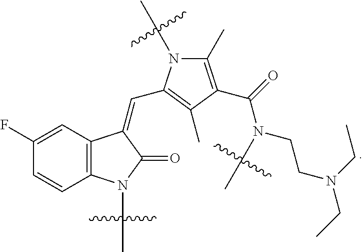 [SMILES: *N(CCN(CC)CC)C(=O)c1c(C)c(/C=C2\C(=O)N(*)c3ccc(F)cc32)n(*)c1C]

In some embodiments, the therapeutic agent component is trametinib (e.g., MEKINIST®) or an analog of trametinib:

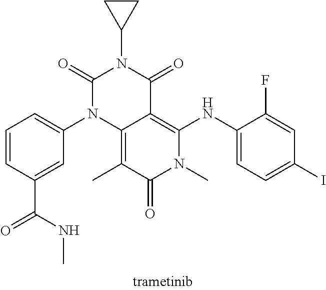 [SMILES: CNC(=O)c1cccc(-n2c(=O)n(C3CC3)c(=O)c3c(Nc4ccc(I)cc4F)n(C)c(=O)c(C)c32)c1]

In some embodiments, a trametinib component has the structure

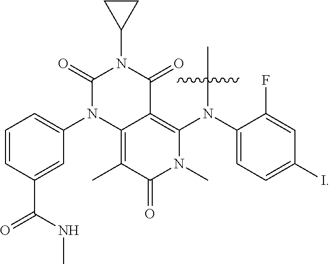 [SMILES: *N(c1ccc(I)cc1F)c1c2c(=O)n(C3CC3)c(=O)n(-c3cccc(C(=O)NC)c3)c2c(C)c(=O)n1C]

In some embodiments, a trametinib component has the structure

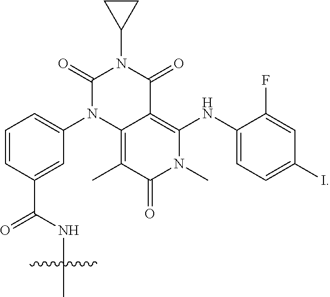 [SMILES: *NC(=O)c1cccc(-n2c(=O)n(C3CC3)c(=O)c3c(Nc4ccc(I)cc4F)n(C)c(=O)c(C)c32)c1]

In some embodiments, a trametinib component has the structure

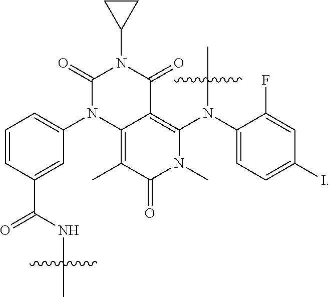 [SMILES: *NC(=O)c1cccc(-n2c(=O)n(C3CC3)c(=O)c3c(N(*)c4ccc(I)cc4F)n(C)c(=O)c(C)c32)c1]

In some embodiments, the therapeutic agent component is warfarin (e.g., COUMADIN®, JANTOVEN®) or an analog of warfarin:

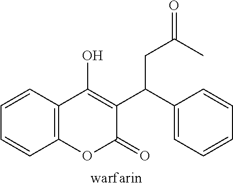 [SMILES: CC(=O)CC(c1ccccc1)c1c(O)c2ccccc2oc1=O]

In some embodiments, a warfarin component has the structure

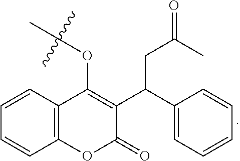 [SMILES: *Oc1c(C(CC(C)=O)c2ccccc2)c(=O)oc2ccccc12]

In some embodiments, the therapeutic agent component is daclatasvir (e.g., DAKLINZA®) or an analog of daclatasvir:

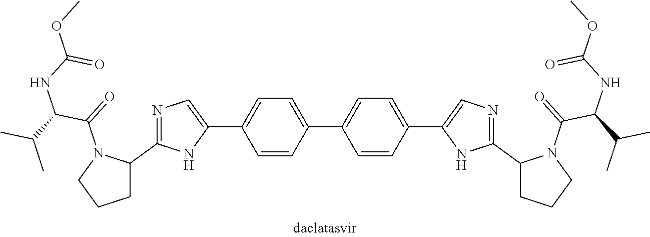 [SMILES: COC(=O)N[C@H](C(=O)N1CCCC1c1ncc(-c2ccc(-c3ccc(-c4cnc(C5CCCN5C(=O)[C@@H](NC(=O)OC)C(C)C)[nH]4)cc3)cc2)[nH]1)C(C)C]

As daclatasvir is a symmetrical drug, many multi-conjugate structures are envisioned with up to at least four cannabinoid components linked to the parent drug. In some embodiments, a daclatasvir component has a cannabinoid component linked at one or more of sites (a), (b), (c), (d), (e), and (f), illustrated below, in any combination:

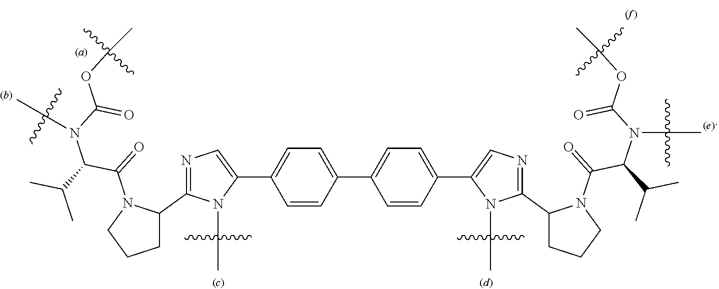 [SMILES: *OC(=O)N(*)[C@H](C(=O)N1CCCC1c1ncc(-c2ccc(-c3ccc(-c4cnc(C5CCCN5C(=O)[C@H](C(C)C)N(*)C(=O)O*)n4*)cc3)cc2)n1*)C(C)C]

In some embodiments, a cannabinoid component is linked at site (a).

In some embodiments, a cannabinoid component is linked at site (a) and site (b). In some embodiments, a cannabinoid component is linked at site (a) and site (c). In some embodiments, a cannabinoid component is linked at site (a) and site (d). In some embodiments, a cannabinoid component is linked at site (a) and site (e). In some embodiments, a cannabinoid component is linked at site (a) and site (f).

In some embodiments, a cannabinoid component is linked at site (a), site (b), and site (c). In some embodiments, a cannabinoid component is linked at site (a), site (b), and site (d). In some embodiments, a cannabinoid component is linked at site (a), site (b), and site (e). In some embodiments, a cannabinoid component is linked at site (a), site (b), and site (f).

In some embodiments, a cannabinoid component is linked at site (a), site (c), and site (d). In some embodiments, a cannabinoid component is linked at site (a), site (c), and site (e). In some embodiments, a cannabinoid component is linked at site (a), site (c), and site (f).

In some embodiments, a cannabinoid component is linked at site (a), site (d), and site (e). In some embodiments, a cannabinoid component is linked at site (a), site (d), and site (f).

In some embodiments, a cannabinoid component is linked at site (a), site (e), and site (f).

In some embodiments, a cannabinoid component is linked at site (a), site (b), site (c), and site (d). In some embodiments, a cannabinoid component is linked at site (a), site (b), site (c), and site (e). In some embodiments, a cannabinoid component is linked at site (a), site (b), site (c), and site (f).

In some embodiments, a cannabinoid component is linked at site (a), site (d), site (d), and site (e). In some embodiments, a cannabinoid component is linked at site (a), site (d), site (d), and site (f).

In some embodiments, a cannabinoid component is linked at site (a), site (d), site (e), and site (f).

In some embodiments, a cannabinoid component is linked at site (a), site (b), site (c), site (d), and site (e). In some embodiments, a cannabinoid component is linked at site (a), site (b), site (c), site (d), and site (f).

In some embodiments, a cannabinoid component is linked at site (a), site (b), site (c), site (d), site (e), and site (f).

In some embodiments, a cannabinoid component is linked at site (b).

In some embodiments, a cannabinoid component is linked at site (b) and site (c). In some embodiments, a cannabinoid component is linked at site (b) and site (d). In some embodiments, a cannabinoid component is linked at site (b) and site (e). In some embodiments, a cannabinoid component is linked at site (b) and site (f).

In some embodiments, a cannabinoid component is linked at site (b), site (c), and site (d). In some embodiments, a cannabinoid component is linked at site (b), site (c), and site (e). In some embodiments, a cannabinoid component is linked at site (b), site (c), and site (f).

In some embodiments, a cannabinoid component is linked at site (b), site (d), and site (e). In some embodiments, a cannabinoid component is linked at site (b), site (d), and site (f).

In some embodiments, a cannabinoid component is linked at site (b), site (e), and site (f).

In some embodiments, a cannabinoid component is linked at site (b), site (c), site (d), and site (e). In some embodiments, a cannabinoid component is linked at site (b), site (c), site (d), and site (f).

In some embodiments, a cannabinoid component is linked at site (b), site (d), site (e), and site (f).

In some embodiments, a cannabinoid component is linked at site (b), site (c), site (d), site (e), and site (f).

In some embodiments, a cannabinoid component is linked at site (c).

In some embodiments, a cannabinoid component is linked at site (c) and site (d). In some embodiments, a cannabinoid component is linked at site (c) and site (e). In some embodiments, a cannabinoid component is linked at site (c) and site (f).

In some embodiments, a cannabinoid component is linked at site (c), site (d), and site (e). In some embodiments, a cannabinoid component is linked at site (c), site (d), and site (f).

In some embodiments, a cannabinoid component is linked at site (c), site (e), and site (f).

In some embodiments, a cannabinoid component is linked at site (c), site (d), site (e), and site (f).

In some embodiments, a cannabinoid component is linked at site (d).

In some embodiments, a cannabinoid component is linked at site (d) and site (e). In some embodiments, a cannabinoid component is linked at site (d) and site (f).

In some embodiments, a cannabinoid component is linked at site (d), site (e), and site (f).

In some embodiments, a cannabinoid component is linked at site (e).

In some embodiments, a cannabinoid component is linked at site (e) and site (f).

In some embodiments, a cannabinoid component is linked at site (f).

In some embodiments, the therapeutic agent component is etoposide (e.g., ETOPOPHOS®, TOPOSAR®) or an analog of etoposide:

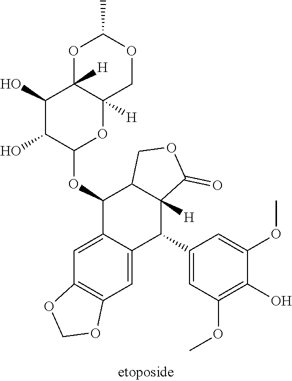

In some embodiments, an etoposide component has the structure

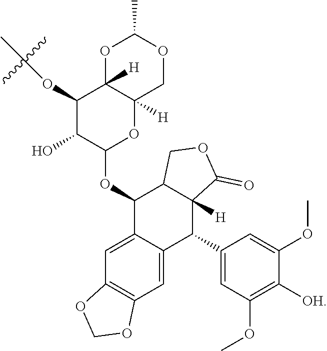

In some embodiments, an etoposide component has the structure

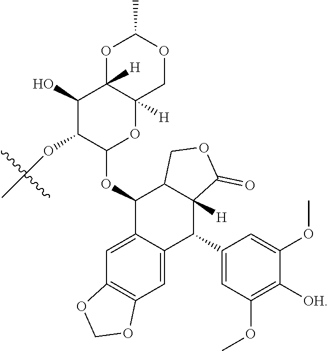

In some embodiments, an etoposide component has the structure

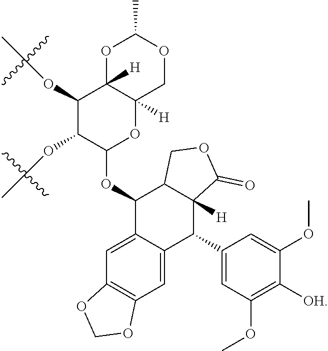

In some embodiments, an etoposide component has the structure

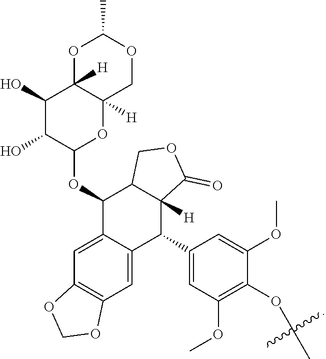

In some embodiments, an etoposide component has the structure

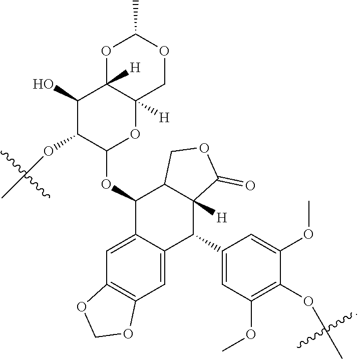

In some embodiments, an etoposide component has the structure

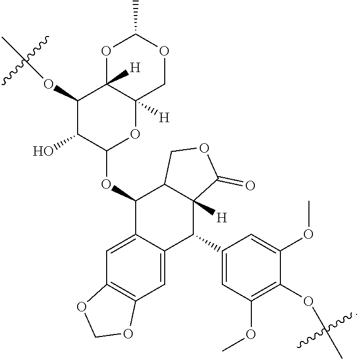

In some embodiments, an etoposide component has the structure

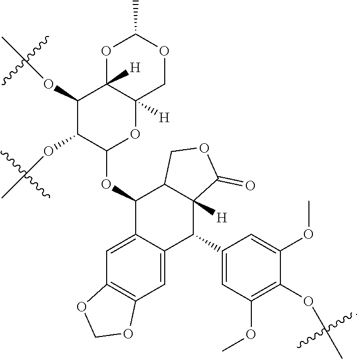

In some embodiments, the therapeutic agent component is atazanavir (e.g., REYATAZ®) or an analog of atazanavir:

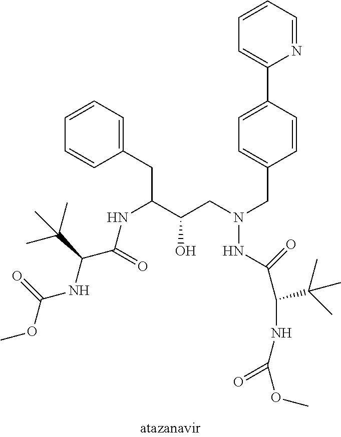 [SMILES: COC(=O)N[C@H](C(=O)NC(Cc1ccccc1)[C@@H](O)CN(Cc1ccc(-c2ccccn2)cc1)NC(=O)[C@@H](NC(=O)OC)C(C)(C)C)C(C)(C)C]

Either or both carbamates in atazanavir may be linked to a cannabinoid component in addition to the OH group or, potentially, the NH hydrazinyl group. In some embodiments, an atazanavir component has the structure

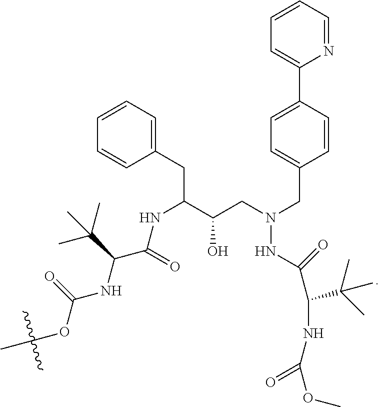 [SMILES: *OC(=O)N[C@H](C(=O)NC(Cc1ccccc1)[C@@H](O)CN(Cc1ccc(-c2ccccn2)cc1)NC(=O)[C@@H](NC(=O)OC)C(C)(C)C)C(C)(C)C]

In some embodiments, an atazanavir component has the structure

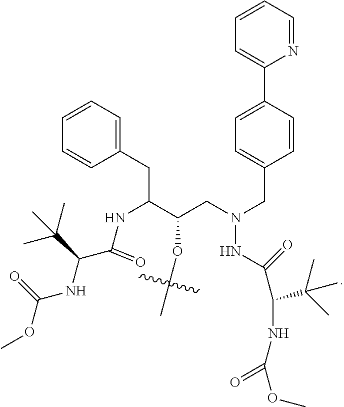 [SMILES: *O[C@@H](CN(Cc1ccc(-c2ccccn2)cc1)NC(=O)[C@@H](NC(=O)OC)C(C)(C)C)C(Cc1ccccc1)NC(=O)[C@@H](NC(=O)OC)C(C)(C)C]

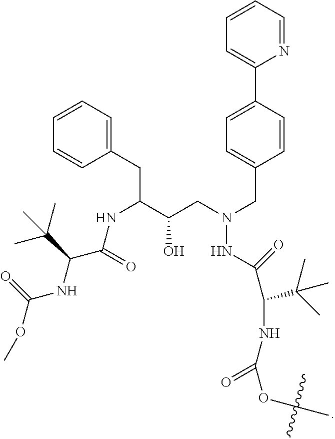 [SMILES: *OC(=O)N[C@H](C(=O)NN(Cc1ccc(-c2ccccn2)cc1)C[C@H](O)C(Cc1ccccc1)NC(=O)[C@@H](NC(=O)OC)C(C)(C)C)C(C)(C)C]

In some embodiments, an atazanavir component has the structure

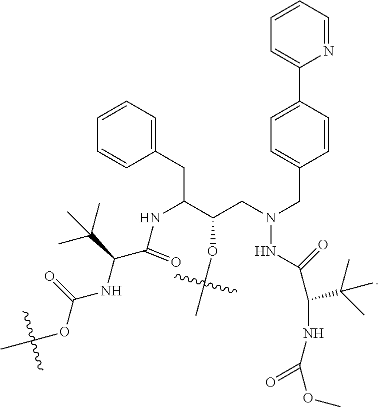 [SMILES: *OC(=O)N[C@H](C(=O)NC(Cc1ccccc1)[C@H](CN(Cc1ccc(-c2ccccn2)cc1)NC(=O)[C@@H](NC(=O)OC)C(C)(C)C)O*)C(C)(C)C]

In some embodiments, an atazanavir component has the structure

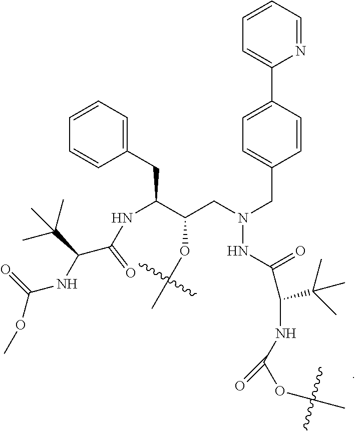 [SMILES: *OC(=O)N[C@H](C(=O)NN(Cc1ccc(-c2ccccn2)cc1)C[C@H](O*)[C@H](Cc1ccccc1)NC(=O)[C@@H](NC(=O)OC)C(C)(C)C)C(C)(C)C]

In some embodiments, an atazanavir component has the structure

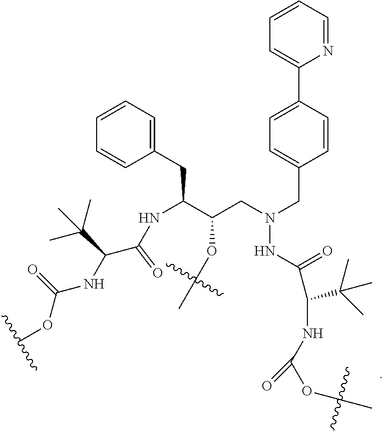 [SMILES: *OC(=O)N[C@H](C(=O)N[C@@H](Cc1ccccc1)[C@H](CN(Cc1ccc(-c2ccccn2)cc1)NC(=O)[C@@H](NC(=O)O*)C(C)(C)C)O*)C(C)(C)C]

In some embodiments, the therapeutic agent component is pravastatin (e.g., PRAVACHOL®) or an analog of pravastatin:

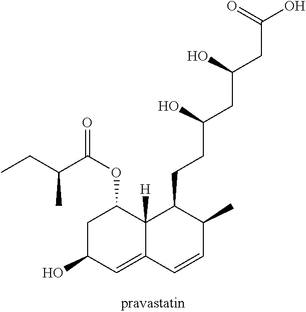 [SMILES: CC[C@H](C)C(=O)O[C@H]1C[C@H](O)C=C2C=C[C@H](C)[C@H](CC[C@@H](O)C[C@@H](O)CC(=O)O)[C@H]21]

Any or all of the three hydroxyl groups and the carboxylic acid group can be linked to a cannabinoid component. In some embodiments, a pravastatin component has one of the following structures

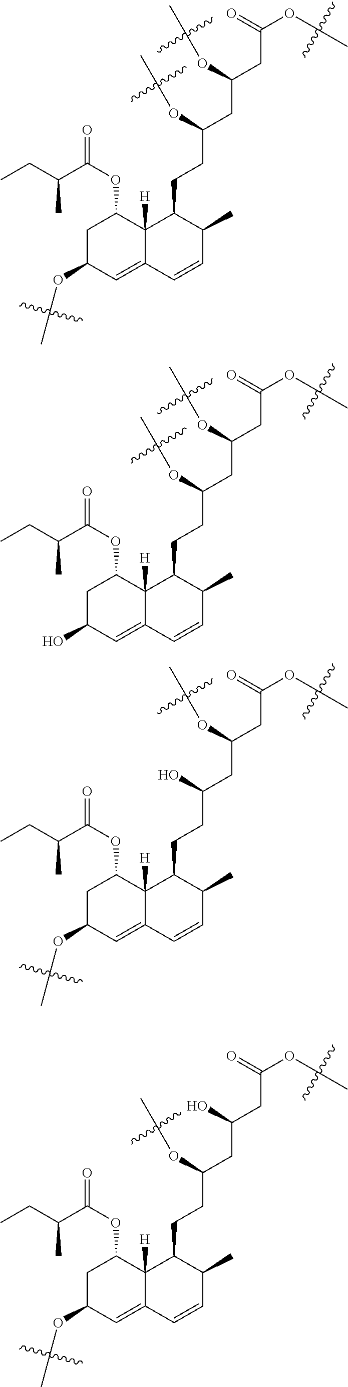 [SMILES: *OC(=O)C[C@@H](C[C@@H](CC[C@@H]1[C@@H]2C(=C[C@@H](O*)C[C@@H]2OC(=O)[C@@H](C)CC)C=C[C@@H]1C)O*)O* ; *OC(=O)C[C@@H](C[C@@H](CC[C@@H]1[C@@H]2C(=C[C@@H](O)C[C@@H]2OC(=O)[C@@H](C)CC)C=C[C@@H]1C)O*)O* ; *OC(=O)C[C@@H](C[C@H](O)CC[C@@H]1[C@@H]2C(=C[C@@H](O*)C[C@@H]2OC(=O)[C@@H](C)CC)C=C[C@@H]1C)O* ; *OC(=O)C[C@H](O)C[C@@H](CC[C@@H]1[C@@H]2C(=C[C@@H](O*)C[C@@H]2OC(=O)[C@@H](C)CC)C=C[C@@H]1C)O*]

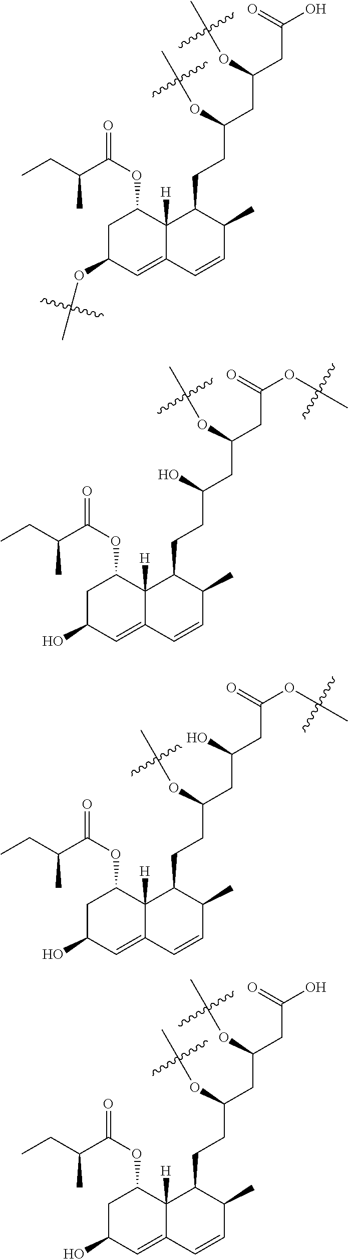 [SMILES: *O[C@H](CC[C@@H]1[C@@H]2C(=C[C@@H](O*)C[C@@H]2OC(=O)[C@@H](C)CC)C=C[C@@H]1C)C[C@H](CC(=O)O)O* ; *OC(=O)C[C@@H](C[C@H](O)CC[C@@H]1[C@@H]2C(=C[C@@H](O)C[C@@H]2OC(=O)[C@@H](C)CC)C=C[C@@H]1C)O* ; *OC(=O)C[C@H](O)C[C@@H](CC[C@@H]1[C@@H]2C(=C[C@@H](O)C[C@@H]2OC(=O)[C@@H](C)CC)C=C[C@@H]1C)O* ; *O[C@H](CC[C@@H]1[C@@H]2C(=C[C@@H](O)C[C@@H]2OC(=O)[C@@H](C)CC)C=C[C@@H]1C)C[C@H](CC(=O)O)O*]

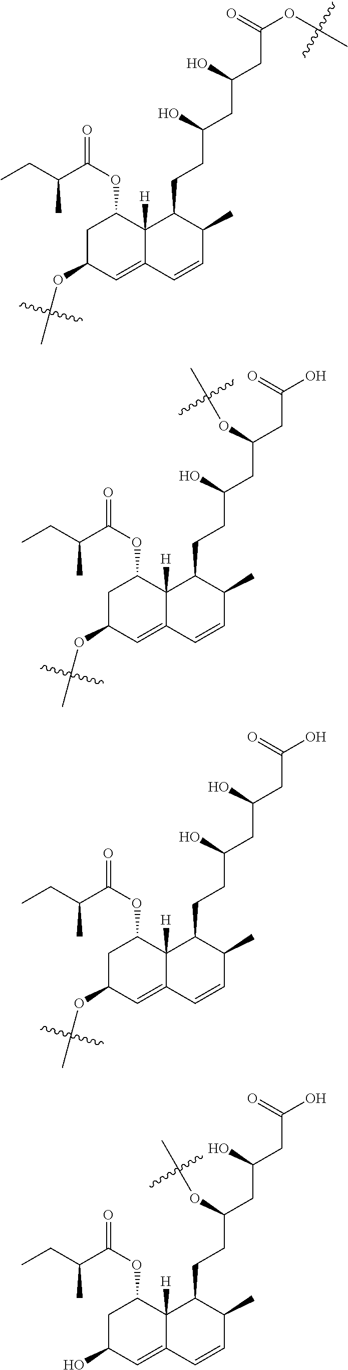 [SMILES: *OC(=O)C[C@H](O)C[C@H](O)CC[C@@H]1[C@@H]2C(=C[C@@H](O*)C[C@@H]2OC(=O)[C@@H](C)CC)C=C[C@@H]1C ; *O[C@@H](CC(=O)O)C[C@H](O)CC[C@@H]1[C@@H]2C(=C[C@@H](O*)C[C@@H]2OC(=O)[C@@H](C)CC)C=C[C@@H]1C ; *O[C@@H]1C=C2C=C[C@H](C)[C@H](CC[C@@H](O)C[C@@H](O)CC(=O)O)[C@H]2[C@@H](OC(=O)[C@@H](C)CC)C1 ; *O[C@H](CC[C@@H]1[C@@H]2C(=C[C@@H](O)C[C@@H]2OC(=O)[C@@H](C)CC)C=C[C@@H]1C)C[C@@H](O)CC(=O)O]

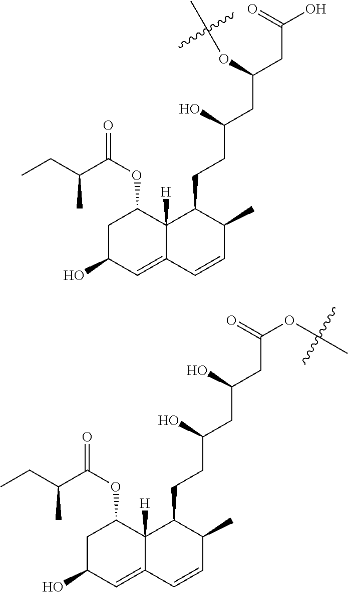 [SMILES: *O[C@@H](CC(=O)O)C[C@H](O)CC[C@@H]1[C@@H]2C(=C[C@@H](O)C[C@@H]2OC(=O)[C@@H](C)CC)C=C[C@@H]1C ; *OC(=O)C[C@H](O)C[C@H](O)CC[C@@H]1[C@@H]2C(=C[C@@H](O)C[C@@H]2OC(=O)[C@@H](C)CC)C=C[C@@H]1C]

In some embodiments, the therapeutic agent component is dasatinib (e.g., SPRYCEL®) or an analog of dasatinib:

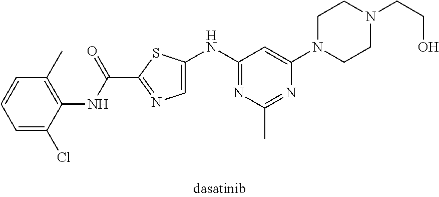 [SMILES: Cc1nc(Nc2cnc(C(=O)Nc3c(C)cccc3Cl)s2)cc(N2CCN(CCO)CC2)n1]

In some embodiments, a dasatinib component has the structure

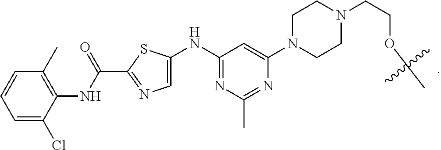 [SMILES: *OCCN1CCN(c2cc(Nc3cnc(C(=O)Nc4c(C)cccc4Cl)s3)nc(C)n2)CC1]

In some embodiments, a dasatinib component has the structure

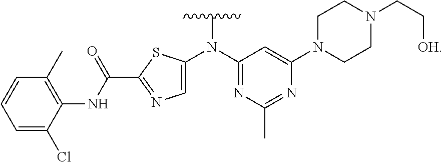 [SMILES: *N(c1cc(N2CCN(CCO)CC2)nc(C)n1)c1cnc(C(=O)Nc2c(C)cccc2Cl)s1]

In some embodiments, a dasatinib component has the structure

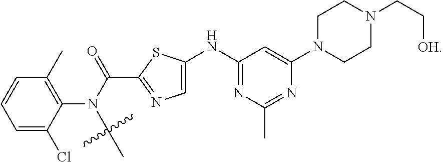 [SMILES: *N(C(=O)c1ncc(Nc2cc(N3CCN(CCO)CC3)nc(C)n2)s1)c1c(C)cccc1Cl]

In some embodiments, a dasatinib component has the structure

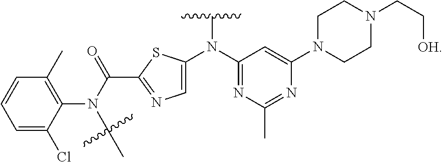 [SMILES: *N(C(=O)c1ncc(N(*)c2cc(N3CCN(CCO)CC3)nc(C)n2)s1)c1c(C)cccc1Cl]

In some embodiments, a dasatinib component has the structure

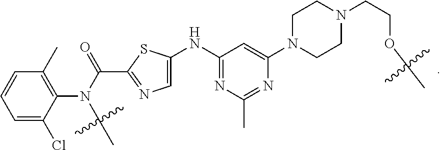 [SMILES: *OCCN1CCN(c2cc(Nc3cnc(C(=O)N(*)c4c(C)cccc4Cl)s3)nc(C)n2)CC1]

In some embodiments, a dasatinib component has the structure

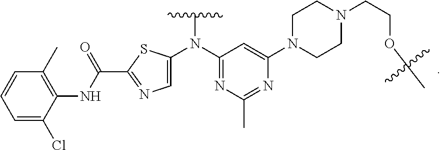 [SMILES: *OCCN1CCN(c2cc(N(*)c3cnc(C(=O)Nc4c(C)cccc4Cl)s3)nc(C)n2)CC1]

In some embodiments, a dasatinib component has the structure

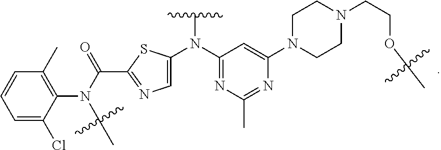 [SMILES: *OCCN1CCN(c2cc(N(*)c3cnc(C(=O)N(*)c4c(C)cccc4Cl)s3)nc(C)n2)CC1]

In some embodiments, the therapeutic agent component is didanosine (e.g., VIDEX®) or an analog of didanosine:

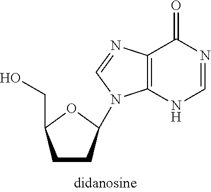

In some embodiments, a didanosine component has the structure

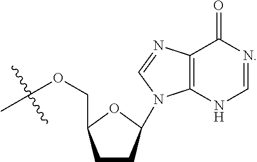

In some embodiments, a didanosine component has the structure

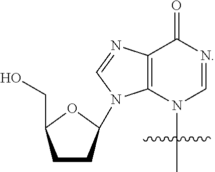

In some embodiments, a didanosine component has the structure

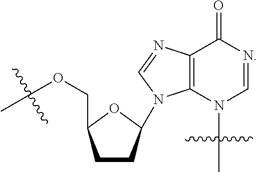

In some embodiments, the therapeutic agent component is stavudine (e.g., ZERIT®) or an analog of stavudine:

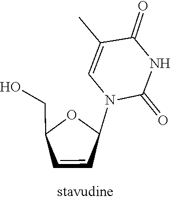 [SMILES: Cc1cn([C@H]2C=C[C@@H](CO)O2)c(=O)[nH]c1=O]

In some embodiments, a stavudine component has the structure

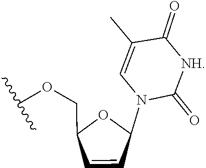 [SMILES: *OC[C@@H]1C=C[C@H](n2cc(C)c(=O)[nH]c2=O)O1]

In some embodiments, a stavudine component has the structure

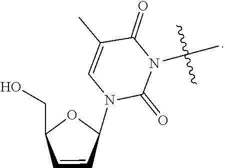 [SMILES: *n1c(=O)c(C)cn([C@H]2C=C[C@@H](CO)O2)c1=O]

In some embodiments, a stavudine component has the structure

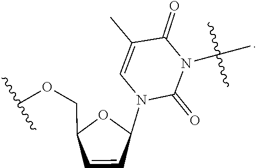 [SMILES: *OC[C@@H]1C=C[C@H](n2cc(C)c(=O)n(*)c2=O)O1]

Additional therapeutic agents can be conjugated as described above. Examples are shown in Table 1.

TABLE 1

| Therapeutic agent(s) | Example Brand Name(s) | Therapeutic Use(s) | Conjugation Options |
|---|---|---|---|
| Aceclofenac | ACECLOFENAC ® | musculoskeletal system | NH and/or COOH group |
| Aceclofenac + Paracetamol | ALTRAFLAM-P ® | anti-inflammatory, | Aceclofenac, NH and/or |
| | | analgesic, anti-pyretic | COOH group Paracetamol |
| | | | (acetaminophen), NH and/or OH |
| Aceclofenac + | ALTRADAY ® | anti-inflammatory, | Aceclofenac, NH and/ |
| Rabeprazole sodium | | analgesic + | or COOH group |
| | | anti-peptic ulcerant | |
| Aclidinium bromide | BRETARIS ® | respiratory system | OH |
| Almotriptan | AMIGNUL ® | nervous system | NH |
| Ambroxol + Theophylline | ACEBROPHYLLINE ® | respiratory system | Ambroxol, OH and/ |
| | | | or NH and/or NH₂; |
| | | | theophyllinc, the NH group |
| Amcinonide | AMCIDERM ® | dermatological | OH |
| Amlodipine | ASTUDAL ® | cardiovascular system | NH₂ and/or replace methyl |
| | | | ester with cannabinoid ester |
| Amlodipine + Atorvastatin | ASTUCOR ® | cardiovascular system | Amlodipine, NH and/or NH₂; |
| | | | atorvastatin, NH and/or |
| | | | OH and/or COOH |
| Amlodipine + Atenolol | AMLOBET ® | cardiology | Amlodipine, NH and/or NH₂; |
| | | | Atenolol, NH and/or OH and/or NH₂ |
| Amlodipine + Metoprolol | CARDIBETA ® AM | anti-hypertensive | Amlodipine, NH and/or NH₂ |
| Amlodipine + | OLMEZEST ® AM | cardiology | Amlodipine, NH and/or NH₂ |
| Olmesartan medoxiomil | | | |
| Amlodipine + Metoprolol tartrate | PROLOMET ® AM 50 | cardiology | Amlodipine, NH and/or NH₂ |
| Amlodipine + Lorsartan potassium | REPLACE-A ® | cardiology | Amlodipine, NH and/or NH₂ |
| Amlodipine + Telmisartan | TELEACT ® AM | anti-hypertensive | Amlodipine, NH and/or NH₂ |
| Amlodipine + Telmisartan + | TELEACT ® TRIO | anti-hypertensive | Amlodipine, NH and/or NH₂ |
| Hydrochlorthiazide | | | |
| Amlodipine + Lorsartan | TRILOPACE ® | cardiology | Amlodipine, NH and/or NH₂ |
| potassium usp + | | | |
| Hydrochlorothiazide ip | | | |
| Arginin + Omithin + | POLILEVO ® | alimentary tract | amino acids at either or both NH₂ |
| Vitamin B6 | | and metabolism | and/or COOH; conjugate Vit B6 at |
| | | | either or both OH and/or P-OH |
| Atenolol | ATENOLOL ® | cardiovascular system | NH and/or OH and/or NH₂ |
| Atenolol + Clortalidone | BLOKIUM-DIU ® | cardiovascular system | either component at NH and/or OH |
| Atenolol + Nifedipine usp | BETATROP ® | cardiology | Atenolol, NH and/or OH and/or NH₂ |
| Atenolol + Lercanidipine usp | LOTENSYL-AT ® | cardiology | Atenolol, NH and/or OH and/or NH₂ |
| Atenolol + Losartan | REPALOL ® H | cardiology | Atenolol, NH and/or OH and/or NH₂ |
| potassium usp | | | |
| Atenolol + Lorsartan potassium | REPALOL ® | cardiology | Atenolol, NH and/or OH and/or NH₂ |
| Azelaic acid | FINACEA ® | inflammatory papules | at either or both end OH groups |
| | | and pustules of mild | |
| | | to moderate rosacea. | |
| Balsalazide | PREMID ® | alimentary tract | OH and/or NH and/or either |
| | | and metabolism | or both COOH groups |
| Betamethasone | DIPROVATE ® PLUS | topical steroid | at any 1, 2, or 3 OH groups |
| Betamethasone dipropionate | DIPROVATE ® RD | topical steroid | at any 1, 2, or 3 OH groups |
| Betamethasone dipropionate + | DIPROVATE ® PLUS ES | topical steroid + | Betamethasone, at any 1, 2, |
| Salicylic acid | | keratolytic | or 3 OH groups; Salicyclic acid, |
| | | | the acid and/or at the OH group |
| Betamethasone dipropionate + | DIPROVATE ® PLUS N | topical steroid | Betamethasone, at any |
| Neomycin sulphate | | | 1, 2, or 3 OH groups |
| Betamethasone dipropionate + | DIPROVATE ® PLUS G | topical steroid | Betamethasone, at any |
| Gentamicin | | | 1, 2, or 3 OH groups |
| Calcipotriene | DOVONEX ® | plaque psoriasis | at any 1, 2, or 3 OH groups |
| Calcipotriene + Betamethasone | ENSTILAR ®, | plaque psoriasis | Betamethasone, at any 1, 2, |
| dipropionate | TALCONEX ® | | or 3 OH groups; calcipotriene at |
| | | | any 1, 2, or 3 OH groups |
| Calcitonin | CALCITONINA ® | systemic hormonal | at any 1 or more NH groups |
| | | preparations | |
| Candesartan cilexetil | PARAPRES ® | cardiovascular system | at acid and/or at NH on tetrazole |
| Candesartan cilexetil + | PARAPRES ® PLUS | cardiovascular system | Candesartan cilexetil, at |
| Hydrochlorothiazide | | | acid and/or at NH on tetrazole; |
| | | | Hydrochlorothiazide, |
| | | | at either or both NH and/or NH₂ |
| Capecitabine | CAXETA ® | cancer | either or both OH and/or NH |
| Cathocisteine | MUCOACTIOL ® | respiratory system | NH₂ and/or either or both |
| | | | of the two acid groups |
| Carboxymethylcysteine | CARBOXIMETILCISTEINE ® | respiratory system | NH₂ and/or either of two acid groups |
| Carfilzomib | KYPROLIS ® | cancer; proteosome | any 1, 2, 3, or 4 NH groups |
| | | inhibitor | |
| Centella asiatica + | BLASTOESTIMULINA | genitourinary system | Metronidazole, at OH |
| Metronidazole + Miconazole | ÓVULOS ® | and sex hormones | |
| Centella asiatica + Neomycin | BLASTOESTIMULINA ® | dermatological | Neomycin, any one or more |
| | | | OH and/or NH₂ groups |
| Ciclopirox + | CICLOPOLI ® | dermatological | Ciclopirox, OH |
| Hydroxypropyl chitosan | | | |
| Ciclopirox olamine | SELERGO ® | dermatological | Ciclopirox, OH |
| Cinitapride | CIDINE ® | alimentary tract | and/or NH₂ |
| | | and metabolism NE | |
| Clebopride | CLEBORIL ® | alimentary tract | and/or NH₂ |
| | | and metabolism NH | |
| Clebopride + Simeticone | FLATORIL ® | alimentary tract | Clebopride, NH and/or NH₂ |
| | | and metabolism | |
| Clindamycin phosphate + | VELTIN ® | dermatological | Clindamycin, to any 1, 2, or |
| Tretinoin | | | 3 OH and/or to the NH and/or |
| | | | to the acid group of tretinoin. |
| Dapsone | ACZONE ® | dermatological | to either or both acids |
| Delgocitinib (LP0133) | | atopic dermatitis | at NH |
| | | (proposed) | |
| Delta-9-tetrahydrocannabinol | SATIVEX ® | nervous system | as described below for |
| (THC) + Cannabidiol (CBD) | | | cannabinoid components |
| Desonide | DESONATE ® | atopic dermatitis | either or both OH and/ |
| | | | or carboxy groups |
| Dihydroergocriptine | ALFA | nervous system | NE or OH |
| | DIHYDROERGOCRYP ® | | |
| Dihydroergocristine | DIEMIL ® | cardiovascular system | Dihydroergocristine, NH and/or |
| mesylate + Piracetam | | | OH; piracetam, NH₂ |
| Dihydroergocryptine | ALMIRID-CRIPAR ® | nervous system | OH and/or NH |
| Dimethyl fumarate | SKILARENCE ® | dermatological | by replacing at least one methyl |
| | | | ester as a cannabinoid ester |
| Doxazosin | PROGANDOL ® | cardiovascular system | NH₂ |
| Doxycycline hyclate | ACTICLATE ® | dermatological | Acticlate (doxycycline), |
| | | | any 1, 2, 3, 4, or 5 OH groups |
| | | | and/or at the amide |
| Ebastine + Pseudoephedrine | RINO-BACTIL ® | respiratory system | pseudoephedrine, NH and/or OH |
| Eflornithine | VANIQA ® | dermatological | at either or both NH₂ and/or to COOH |
| Eplerenone | ELECOR ® | cardiovascular system | by replacing a methyl ester |
| | | | with a cannabinoid ester |
| Erythromycin | AKNE-MYCIN ® | dermatological | at any 1, 2, 3, 4, or 5 OH groups |
| Erythromycin + Tretinoin | AKNEMYCIN PLUS | dermatological | Erythromycin, at any 1, 2, 3, 4, or |
| | | | 5 OH groups; tretinoin, at its acid |
| Erythromycin + Zinc | ZINERYT ® | dermatological | Erythromycin, at any 1, 2, 3, 4, or |
| acetate dihydrate | | | 5 OH groups |
| Etodolac | LODINE ® | dermatological | via acid group |
| Flupredniden-21-acetate + | SALI-DECODERM ® | dermatological | Flupredniden-₂1-acetate, either or |
| Salicylic acid | | | both OH groups; salicylic acid at the |
| | | | acid and/or at the OH group |
| Fluprednidene acetate | DECODERM ® | dermatological | either or both OH groups |
| Fluprednidene acetate + | CRINOHERMAL ® | genitourinary system | conjugate either or both components |
| Estradiol | | and sex hormones | at either or both OH groups |
| Fluprednidene acetate + | DECODERM ® TRI | dermatological | Fluprednidene, either |
| Miconazole nitrate | CREAM | | or both OH groups |
| Flurandrenolide USP | CORDRAN ® | dermatological | either or both OH groups |
| Flurbiprofen | CEBUTID ® | musculoskeletal system | acid |
| Gelatin powder + Biotin | GELACET PULVER ® | alimentary tract | Biotin at acid and/ |
| | | and metabolism | or possible NH |
| Gentamicin sulfate | REFOBACIN ® | dermatological | any 1, 2, or 3 OH groups and/ |
| | | | or any 1, 2, 3, or 4 NH₂ |
| | | | groups and/or one NH group |
| Ginko biloba + Coenzyme | CLEVIA ® | alimentary tract | Vitamin B₂, NH and/or any |
| Q10 + Vitamin B2 + | | and metabolism | 1, 2, 3, or 4 OH groups |
| Commiphora mirra | | | |
| Glucosamine | CODEROL ® | musculoskeletal system | NH₂ and/or any 1, 2, 3, |
| | | | or 4 OH groups |
| Hyaluronic acid + Hop extract + | GYNOMUNAL ® | genitourinary system | vitamin E via the OH group |
| Liposomes + Vitamin E | | and sex hormones | |
| Hydrocortisone + Urea | HYDRODEXAN ® | dermatological | any 1, 2, or 3 OH groups |
| Hydrocortisone acetate | | dermatological | either or both OH groups |
| Hydrocortisone butyrate | LATICORT ® | dermatological | either or both OH groups |
| Ibrutinib | IMBRUVICA ® | B cell cancers | NH₂ |
| Ingenol mebutate | PICATO ® | topical treatment | any 1, 2, or 3 OH groups |
| | | of actinic keratosis. | |
| Isotretinoin | AKNENORMIN ® | dermatological | at its acid |
| Lorazepam | SERENASE ® | nervous system | OH and/or NH |
| Meptazinol | MEPTID ® | nervous system | OH |
| Methocarbamol | ROBAXIN ® | musculoskeletal system | OH and/or via the carbamate |
| Minocycline | AKNEMIN ® | anti-infective for | any 1, 2, 3, 4, or 5 OH groups |
| | | systemic use | and/or at the amide |
| Mometasone furoate | IVOXEL ® | dermatological | OH |
| Mupirocin | MUPIDERM ® | dermatological | any 1, 2, or 3 OH groups |
| | | | and/or at COOH |
| Naproxen sodium salt | SYNFLEX ® | musculoskeletal system | COOH |
| Nifuratel + Nystatin | DAFNEGIL ® | genitourinary system | nystatin, COOH and/or NH₂, |
| | | and sex hormones | and/or any 1, 2, 3, 4, 5, 6, |
| | | | 7, 8, 9, or 10 OH groups |
| Noretisterone | ELASTOLABO ® | genitourinary system | OH |
| | | and sex hormones | |
| Nystatin | CANDIO-HERMAL ® | dermatological | COOH and/or NH₂, and/or any |
| | | | 1, 2, 3, 4, 5, 6, 7, 8, 9, |
| | | | or 10 OH groups |
| Octopirox | MYFUNGAR ® | dermatological | N-OH moiety |
| Paracetamol | FEBRECTAL ® | nervous system | Paracetamol (acetaminophen), |
| | | | NH and/or OH |
| Paracetamol + Codein + | ALGIDOL ® | nervous system | Paracetamol, OH and/or |
| Ascorbic acid | | | NH; codeine, OH; ascorbic |
| | | | acid, any 1, 2, 3, or 4 OH groups |
| Phenol-methanal- | TANNOSYNT | dermatological | OH of phenol |
| urea polycondensate | LOTION ® | | |
| Pidotimod | POLIMOD ® | immunostimulant | acid and/or the amide |
| Piketoprofen | CALMATEL ® | musculoskeletal system | NH |
| Piracetam | METADIEMIL ® | cardiovascular system, | NH₂ |
| | | nervous system | |
| Piroctone olamine + | LYGAL DUO ® | dermatological | N-OH |
| Climbazol | | | |
| Potassium azeloyl | ROZERO ® | dermatological | Potassium azeloyl diglycinate |
| diglycinate + Vitamin E + | | | via either or both acid groups; |
| Hydroxypropyl chitosan | | | vitamin E via the OH group |
| Prednisolon + | LYGAL ® | dermatological | Prednisolon, any 1, 2, or 3 |
| Piroctone olamine | | | OH groups; piroctone, N-OH |
| Pyrithion-zink | DE-SQUAMAN | dermatological | conjugate via the |
| | HERNIAL ® | | SH or OH form |
| Retapamulin | ALTABAX ® | dermatological | OH |
| Retinol (Vitamin A) | GELACET ® | alimentary tract | OH |
| | | and metabolism | |
| Rosuvastatin | CRESTOR ® | cardiovascular system | COOH and/or either or |
| | | | both of two OH groups |
| Salicyclic acid | SPEELAC ® | anti-acne | acid and/or at OH group |
| Salicylic acid; Sodium lactate; | SOTRET ® SOAP | anti-acne | Salicyclic acid, the acid |
| Glycerine; Titanium dioxide; | | | and/or at the OH group |
| Triclosan; E.D.T.A./Codex; | | | |
| Basil extract; Mint Oil/Menthol; | | | |
| Tea tree oil; Olive oil/ Oleivem | | | |
| Sarecycline | SEYSARA ® | dermatological | any 1, 2, 3, or 4 OH |
| | | | groups and/or COOH |
| Silodosin | SILODYX ® | genitourinary system | OH and/or NH and/or NH₂ |
| | | and sex hormones | |
| Sitagliptin | TESAVEL ® | alimentary tract | NH₂ |
| | | and metabolism | |
| Sitagliptin + Metformin | EFFICIB ® | alimentary tract | Sitagliptin, NH₂; metformin, |
| | | and metabolism | NH and/or NH₂ |
| Sorafenib tosylate | | anti-cancer | urea |
| Sulfamethoxazole | SOLTRIM ® | anti-infective for | NH₂ and/or NH |
| | | systemic use | |
| Tacalcitol | CURATODERM ® | dermatological | any 1, 2, or 3 OH groups |
| Tacrolimus | PROTOPIC ® | severe atopic dermatitis | any 1, 2, or 3 OH groups |
| Tannic acid | TANNO-HERMAL ® | dermatological | conjugate to any OH group or |
| | | | combination of OH groups |
| Tazoretene | TAZORAC ® | dermatological | by replacing ethy ester with |
| | | | a cannabinoid ester |
| Tolterodine 1-tartrate | UROTROL ® | genitourinary system | OH |
| | | and sex hormones | |
| Triamterene | PRESTOLE ® | cardiovascular system | any 1, 2, or 3 NH₂ groups |
| Ucp peptide | THIOMUCASE ® | dermatological | NH₂ |
| Urea | AQEO ® | dermatological | NH₂ |
| Urea + Lauromacrogols | BALNEUM ® LOTION | dermatological | Urea, at NH₂ |
| Urea + Polidocanol | OPTIDERM ® CRÈME | dermatological | NH₂ |
| Urea + Sodium lanreth | BALNEUM INTENSIV ® | dermatological | Urea, at NH₂ |
| Venlafaxine hydrochloride | DOBUPAL ® | nervous system | OH |
| Vitamin B1 + Vitamin B6 + | HIDROXIL ® | alimentary tract | Vitamin B1, OH and/or NH₂; |
| Vitamin B12 | | and metabolism | vitamin B6, OH and/or P-OH |
| Vitamin C | FEMINELLA | genitourinary system | any 1, 2, 3, or 4 OH groups |
| | VAGI C ® | and sex hormones | |
| Xanthinol furosemide + | SALIDUR ® | cardiovascular system | Furosemide, conjugate via the |
| Triamterene | | | acid and/or the NH and/or |
| | | | NH₂ groups; Triamterene, |
| | | | any 1, 2, or 3 NH₂ groups |
| Acamprosate calcium | ACAMPROL ® | Neurology and | S-OH or NH |
| | | Psychiatry | |
| Entacapone | ADCAPONE ® | Neurology and | OH |
| | | Psychiatry | |
| Methyl phenidate | ADDWIZE ® | Neurology and | NH |
| hydrochloride vsp | | Psychiatry | |
| S-Adenosyl methionine | ADESAM ® | Neurology and | OH and/or acid and/or |
| | | Psychiatry | one or both NH₂ |
| Adefovir | ADHEB ® | Anti-viral | Conjugate as phosphate |
| | | | cannabinoid ester |
| Adefovir dipivoxil | ADFOV1R ® | Infections | Conjugate as phosphate |
| | | | cannabinoid ester |
| Memantine hydrochloride | ADMENTA ® | Neurology and | NH₂ |
| | | Psychiatry | |
| Doxorubicin hydrochloride | ADVADOX ® | Anti-cancer | any of 1, 2, 3, 4, or 5 |
| | | | OH and/or the NH₂ |
| Epalrestat | ALDORACE ® | Diabetc neuropathy | to acid |
| Fexofenadine | ALTIVA ® | Anti-histamine | either or both OH or to acid |
| Amisulpride | AMIVAL ® | Anti-psychotic | NH₂ or NH |
| Amitriptyline ip, | AMIXIDE ® | Neurology and | chloridiazepoxide, |
| Chloridiazepoxide ip | | Psychiatry | to NH |
| Amlodipine, Atenolol ip | AMLOBET ® | Cardiology | Amlodipine, NH₂; atenolol, |
| | | | OH and/or NH and/or NH₂ |
| Amlodipine | AMLOSUN ® | Cardiology | NH₂ |
| Bicalutamide | ANDROBLOK ® | Cancer | OH |
| Oxazepam | ANXOZAP ® | Neurology and | OH |
| | | Psychiatry | |
| Hydrolchlorothiazide ip | AQUAZIDE ® | Cardiology | NH or NH₂ |
| Aripiprazole | ARPIZOL ® | Neurology and | NH |
| | | Psychiatry | |
| Atomoxetine | ATTENTROL ® | Neurology and | NH |
| | | Psychiatry | |
| Atorvastatin | AZTOR ® | Cardiology | either of both OH groups and/or |
| | | | COOH and/or NH groups |
| Atorvastatin, Aspirin ip | AZTORE ® | Cardiology | Atorvastatin, either or both of |
| | | | two OH groups and/or |
| | | | COOH and/or NH groups |
| Atorvastatin, Ezetimibe | AZTOR ® | Cardiology | Atorvastatin, either or both of |
| | | | two OH groups and/or |
| | | | COOH and/or NH groups |
| Mycophenolate | BAXMUNE ® | Immuno supressant | |
| | | | OH or COOH |
| Propranolol hydrochloride Ip | BETACAP ® | Neurology and | OH or NH |
| | | Psychiatry | |
| Propranolol hydrochloride Ip, | BETACAP ® PLUS | Neurology and | Propanolol, OH or NH |
| Flunarizine | | Psychiatry | |
| Nifedipine usp, Atenolol bp | BETATROP ® | Cardiology | Nifedipine, NH or replace a |
| | | | methyl ester with a |
| | | | cannabinoid ester; atenolol, |
| | | | OH and/or NH and/or NH₂ |
| Betahistine hydrochloride | BETA VERT ® | Nausea | NH |
| Brimonidine tartrate, Timolol, | BRIMOLOL ® | Opthalmology | Brimonidine, NH; timolol, |
| Benzalkonium chloride | | | NH and/or OH |
| Brimonidine tartrate, | BRIMOSUN ® | Opthalmology | Brimonidine, NH |
| Oxychloro complex | | | |
| Brimonidine tartrate | BRIMOSUN ®-P | Opthalmology | NH |
| Bortezomib | VELCADE ® | multiple myeloma and | NH or link as a borate ester. |
| | | mantle cell lymphoma | |
| Paclitaxel | ABRAXANE ® | lung, ovarian, and breast | any OH and/or any NH |
| | | cancer, Kaposi sarcoma | |
| Docetaxel | DOCEFREZ ®, | breast, lung, prostate, stomach, | any OH and/or any NH |
| | TAXOTERE ® | and head and neck cancer | |
| Efavirenz | SUSTIVA ® | HIV | NH |
| Irinotecan | ONIVYDE ®, | cancer of the | OH |
| | CAMPTOSAR ® | colon or rectum | |
| Tenofovir | VEMLIDY ®, | hepatitis B and | link by making a cannabinoid |
| | VIREAD ® | HIV infection | phosphonate ester prodrug |
| Lopinavir | KALETRA ® | HIV | OH and/or any 1,2, or 3 NH |
| Ritonavir | | | OH and/or either or both NH |
| Lamivudine | EPIVIR ® HBV, | hepatitis B and HIV | OH and/or NH₂ |
| | EPIVIR ® | | |
| Zidovudine | RETROVIR ® | HIV | OH and/or NH |
| Nevirapine | VIRAMUNE ® XR, | HIV | NH |
| | VIRAMUNE ® | | |
| Ganciclovir | ZIRGAN ® | cytomegalovirus | either or both OH and/or NH₂ |
| Valacyclovir | VALTREX ® | herpes, chicken pox | either or both NH₂ |
| Ledipasvir | HARVONI ® | hepatitis C | any or all NH |
| | (w/sofubusvir) | | |
| Valganciclovir | VALCYTE | cytomegalovirus | OH and/or either Of both NH₂ |

In any of the embodiments described above in which two or more cannabinoid components can be attached, each cannabinoid component can be the same or different, and, when linkers are used, each linker can be the same or different.

Linkers

In some embodiments, linkers used to connect a therapeutic agent component and a cannabinoid component are typically two to 10 atoms in length and are functionalized to facilitate release of the cannabinoid. In some embodiments, this release may occur approximately when the therapeutic agent engages its biological target.

A variety of linkers can be used in the conjugate molecules. Examples are shown below.

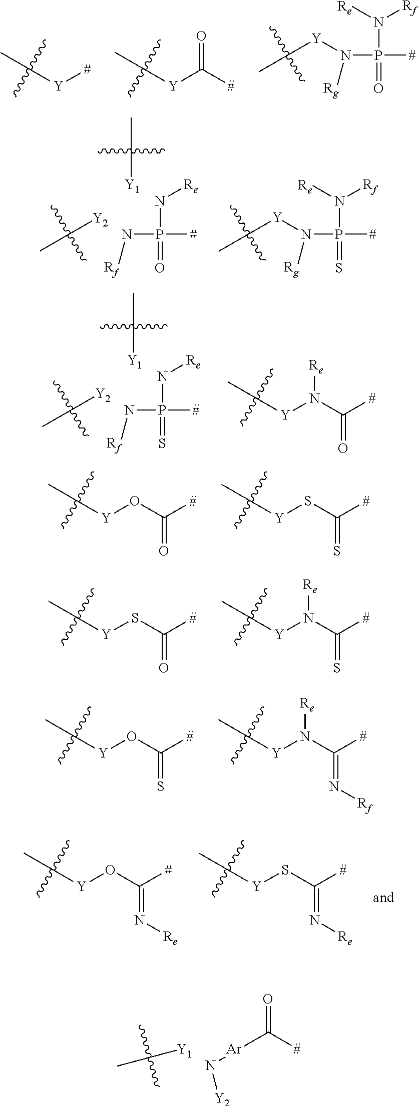

in which [symbol] marks a bond attaching the linker to the therapeutic agent component, # indicates a site of covalent attachment to the cannabinoid component, and in which:
Y, Y₁, and Y₂ independently are absent or Y, Y₁, and Y₂ independently are selected from the group consisting of
   (a) C1-C12 linear or branched alkyl, optionally substituted with
      (1) 1, 2, 3, 4, 5, 6, 7, 8, or 9 fluorine atoms; and/or
      (2) 1, 2, or 3 substituents selected from the Group One Substituents;
   (b) C2-C12 linear or branched alkenyl, optionally substituted with
      (1) 1, 2, 3, 4, 5, 6, 7, 8, or 9 fluorine atoms; and/or
      (2) 1, 2, or 3 substituents selected from the Group One Substituents;
   (c) C1-C12 linear or branched heteroalkyl containing 1, 2, 3, or 4 heteroatoms independently selected from O, N, and S, optionally substituted with
      (1) 1, 2, 3, 4, 5, 6, 7, 8, or 9 fluorine atoms; and/or
      (2) 1, 2, or 3 substituents selected from the Group One Substituents;
   (d) a 6- to 10-membered aromatic, optionally substituted with 1, 2, 3, or 4 substituents independently selected from the group consisting of
      (1) phenyl,
      (2) halide,
      (3) C1-C6 linear or branched alkyl, optionally substituted with
         (i) 1, 2, 3, 4, 5, 6, 7, 8, or 9 fluorine atoms; and/or
         (ii) 1, 2, or 3 substituents independently selected from the Group Two Substituents, and
      (4) C1-C6 linear or branched heteroalkyl containing 1, 2, or 3 atoms independently selected from O, N, and S and optionally substituted with
         (i) 1, 2, 3, 4, 5, 6, 7, 8, or 9 fluorine atoms; and/or
         (ii) 1, 2, or 3 substituents independently selected from the Group Two Substituents;
   (e) a 6- to 10-membered heteroaromatic comprising 1, 2, 3, 4, 5, or 6 heteroatoms independently selected from O, N, and S and optionally substituted with 1, 2, 3, or 4 substituents independently selected from
      (1) phenyl,
      (2) halide,
      (3) trifluoromethyl,
      (4) C1-C6 linear or branched alkyl, optionally substituted with
         (i) 1, 2, 3, 4, 5, 6, 7, 8, or 9 fluorine atoms; and/or
         (ii) 1, 2, or 3 substituents independently selected from the Group Two Substituents, and
      (5) C1-C6 linear or branched heteroalkyl containing 1, 2, or 3 atoms independently selected from O, N, and S and optionally substituted with
         (i) 1, 2, 3, 4, 5, 6, 7, 8, or 9 fluorine atoms; and/or
         (ii) 1, 2, or 3 substituents independently selected from the Group Two Substituents; and
   (f) a C1-C24 linear or branched heteroalkyl containing 1, 2, 3, 4, 5, 6, 7, or 8 heteroatoms independently selected from O, N, and S, optionally substituted with
      (i) 1, 2, 3, 4, 5, 6, 7, 8, or 9 fluorine atoms; and/or
      (ii) 1, 2, 3, 4, 5, or 6 substituents selected from the Group One Substituents;
Ar is either:
   (a) a 6- to 10-membered aromatic, optionally substituted with 1, 2, 3, or 4 substituents independently selected from the group consisting of
      (1) phenyl,
      (2) halide,
      (3) C1-C6 linear or branched alkyl, optionally substituted with
         (i) 1, 2, 3, 4, 5, 6, 7, 8, or 9 fluorine atoms; and/or
         (ii) 1, 2, or 3 substituents independently selected from the Group Two Substituents; or
   (b) a 6- to 10-membered heteroaromatic comprising 1, 2, 3, 4, 5, or 6 heteroatoms independently selected from O, N, and S and optionally substituted with 1, 2, 3, or 4 substituents independently selected from
      (1) phenyl,
      (2) halide,
      (3) trifluoromethyl,
      (4) C1-C6 linear or branched alkyl, optionally substituted with
         (i) 1, 2, 3, 4, 5, 6, 7, 8, or 9 fluorine atoms; and/or
         (ii) 1, 2, or 3 substituents independently selected from the Group Two Substituents, and
      (5) C1-C6 linear or branched heteroalkyl containing 1, 2, or 3 atoms independently selected from O, N, and S and optionally substituted with
         (i) 1, 2, 3, 4, 5, 6, 7, 8, or 9 fluorine atoms; and/or
         (ii) 1, 2, or 3 substituents independently selected from the Group Two Substituents; and
            Rₑ, R_{f}, and R_{g} independently are R as defined above.

In other embodiments, a number of other types of linkers can be used. These linkers include self-cleaving linkers such as acid-labile linkers and protease-labile linkers, linkers comprising negatively charged groups, linkers comprising sugar moieties, and others.

Examples of acid-labile linkers include acetals, hydrazones (including acylhydrazones, hydrazines), imines, esters, linkers containing disulfide bonds, and linkers containing pH-sensitive chelators. See, e.g., Vlahov & Leamon, Bioconjug. Chem. 23, 1357-69, 2012); Xiao et al., Nanoscale 4, 7185-93, 2012; Abu et al., Eur. J. Cancer 48, 2054-65, 2011; DiJoseph et al., Clin Cancer Res. 12, 242-49, 2006; Kale & Torchilin, Bioconjugate Chemistry 18, 363-70, 2007; Sawant et al., Bioconjugate Chemistry 17, 943-49, 2006; Reddy et al., Sci. Rep. 8, 8943, 2018.

Examples of protease-labile linkers include linkers comprising a valine-citrulline bond, 3-glucuronic acid-based linkers, and imides. See, e.g., Weinstain et al., Chem. Commun. (Camb.) 46, 553-55, 2010; Shao et al., Cancer 118, 2986-96, 2010; Liang et al., J. Controlled Release 160, 618-29, 2012; Barthel et al., J. Med. Chem. 55, 6595-607, 2012; Nolting, Methods Mol. Biol. 1045, 71-100, 2013; Erickson, Cancer Res. 66, 4426-33, 2006; Jeffrey et al., Bioconjugate Chem. 17, 831-40, 2006; Dubowchik et al., Bioconjugate Chem. 13, 855-69, 2002; Mhidia et al., Org. Lett. 12, 3982-85, 2010.

Examples of linkers comprising negatively charged groups are disclosed, for example, in Leamon et al., J. Pharm. Exp. Ther. 336, 336-43, 2011.

Examples of linkers containing sugar moieties are disclosed, for example in Mikuni et al., Biol. Pharm. Bull. 31, 1155-58, 2008.

Other types of linkers include thioether-based linkers and N-succinimidyl-4-(N-maleimidylmethyl) cyclohexane-1-carboxylate (SMCC) linker (see, e.g., Juárez-Hernández et al., ACS Med. Chem. Lett. 3, 799-803, 2012) and linkers comprising an acetamide moiety and linkers comprising sulfur-containing amides or esters (Davaran et al., J. Pharm. Pharmacol. 55, 513-17, 2003).

Cannabinoid Component

A “cannabinoid component” as used in this disclosure is that portion of the cannabinoid that is present in the conjugate molecule and covalently attached to the linker, as shown in the examples below.

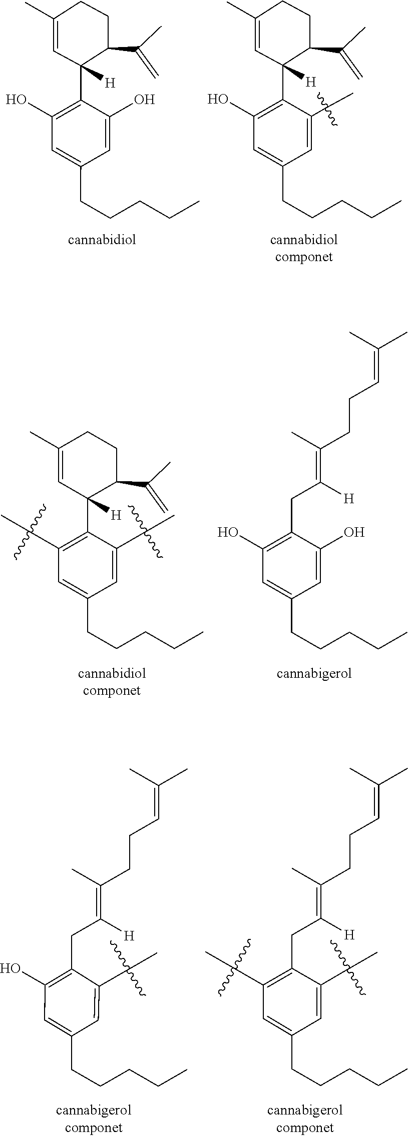 [SMILES: C=C(C)[C@@H]1CCC(C)=C[C@H]1c1c(O)cc(CCCCC)cc1O ; *c1cc(CCCCC)cc(O)c1[C@@H]1C=C(C)CC[C@H]1C(=C)C ; *c1cc(CCCCC)cc(*)c1[C@@H]1C=C(C)CC[C@H]1C(=C)C ; CCCCCc1cc(O)c(C/C=C(\C)CCC=C(C)C)c(O)c1 ; *c1cc(CCCCC)cc(O)c1C/C=C(\C)CCC=C(C)C ; *c1cc(CCCCC)cc(*)c1C/C=C(\C)CCC=C(C)C]

The cannabinoid component can be provided by any cannabinoid that contains a hydroxy group to which the linker can be attached or to which a therapeutic agent component can be covalently attached or a carboxylic acid to which a linker can be connected by way of an ester, amide, or thioester bond. The cannabinoid can be a naturally occurring molecule, either isolated or synthesized, or a modified version of a naturally occurring molecule. See, for example, Morales et al., Frontiers in Pharmacology June 2017 review, 1-18.

Examples of cannabinoids include, but are not limited to, cannabigerols, cannabichromenes, cannabidiols, tetrahydrocannabinols, cannabicyclols, cannabielsoins, cannabinols, cannabinodiols, cannabitriols, dehydrocannabifurans, cannabifurans, cannabichromanons, and cannabiripsols.

Examples of cannabigerols include cannabigerolic acid (CBGA), cannabigerolic acid monomethylether (CBGAM), cannabigerol (CBG), cannabigerol monomethyleither (CBGM), cannabigerovarinic acid (CBGVA), and cannabigerovarin (CBGV).

Examples of cannabichromenes include cannabichromenic acid (CBC), cannabichromene (CBC), cannabichromevarinic acid (CBCVA), and cannabichromevarin (CBCV).

Examples of cannabidiols include cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), and cannabidiorcol (CBD-C₁).

Examples of tetrahydrocannabinols include Δ-9-tetrahydrocannabinolic acid A (THCA-A), Δ-9-tetrahydrocannabinolic acid B (THCA-B), Δ-9-tetrahydrocannabinol (THC), Δ-9-tetrahydrocannabinolic acid-C₄ (THCA-C₄), Δ-9-tetrahydrocannabinol-C4 (THC-C4), Δ-9-tetrahydrocannabivarinic acid (THCVA), Δ-9-tetrahydrocannabivarin (THCV), Δ-9-tetrahydrocannabiorcolic acid (THCA-C₁), Δ-9-tetrahydrocannabiorcol (THC-C₁), Δ-7-cis-tetrahydrocannabivarin, Δ-8-tetrahydrocannabinolic acid (Δ⁸-THCA), and Δ-8-tetrahydrocannabinol (Δ⁸-THC).

Examples of cannabicyclols include cannabicyclolic acid (CBLA), cannabicyclol (CBL), and cannabicyclovarin (CBLV).

Examples of cannabielsoins include cannabielsoic acid A (CBEA-A), cannabielsoic acid B (CBEA-B), and cannabielsoin (CBE).

Examples of cannabinols and cannabinodiols include cannabinolic acid (CBNA), cannabinol (CBN), cannabinol-C₄ (CBN-C₄), cannabivarin (CBV), cannabinol-C₂ (CBN-C₂), cannabiorcol (CBN-C₁), cannabinodiol (CBND), and cannabinodivarin (CBVD).

Examples of cannabitriols include cannabitriol (CBT), 10-ethoxy-9-hydroxy-Δ-6a-tetrahydrocannabinol, cannabitriolvarin (CBTV), and ethoxy-cannabitriolvarin (CBTVE).

Cannabifurans include dehydrocannabifuran (DCBF) and cannabifuran (CBF).

Examples of other cannabinoids include cannabichromanon (CBCN), 10-oxo-Δ-6a-tetrahydrocannabinol (OTHC), cannabiripsol (CBR), and trihydroxy-Δ-9-tetrahydrocannabinol (triOH-THC).

In some embodiments, the cannabinoid component is provided by cannabidiol.

Conjugate Molecules Comprising Two Therapeutic Agent Components

In some embodiments, in which the cannabinoid component has two hydroxyl groups, a second therapeutic agent component can be covalently attached to the second hydroxyl group by means of a second linker such that the conjugate molecule contains a first therapeutic agent component and a second therapeutic agent component covalently attached to the cannabinoid component by means of a first linker and a second linker, respectively.

Conjugate molecules in which at least one of the linkers is

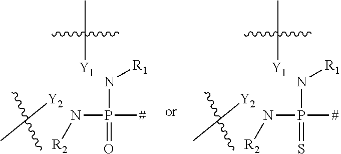

can comprise a second therapeutic agent covalently attached to the linker rather than to the cannabinoid component. In some embodiments, first therapeutic agent component is covalently attached at Y₂. In some embodiments, the first therapeutic agent component is covalently attached at Y₁.

In conjugate molecules comprising two therapeutic agent components, the therapeutic agent components can be the same or different.

Conjugate Molecules Comprising Two Cannabinoid Components

Conjugate molecules in which the therapeutic agent components is

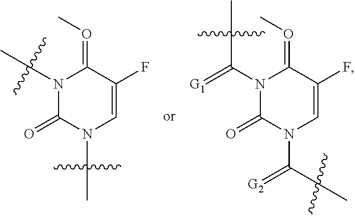

for example, can have a cannabinoid component covalently attached at both nitrogen atoms. In some embodiments, the two cannabinoid components are the same. In some embodiments, the two cannabinoid components are different.

Examples of Conjugate Molecules

In the examples below, “CBN” is a cannabinoid component. Cannabinoid stereochemistry is generally not shown in examples as a reminder that all stereoisomers are allowed. Examples that show stereochemistry do not exclude other isomers. Examples shown include linkers derived from ester, carbonate, and carbamate functionalities. Additional linkers as described above can also be used.

### Conjugate Molecules Comprising Temozolomide Analog Components

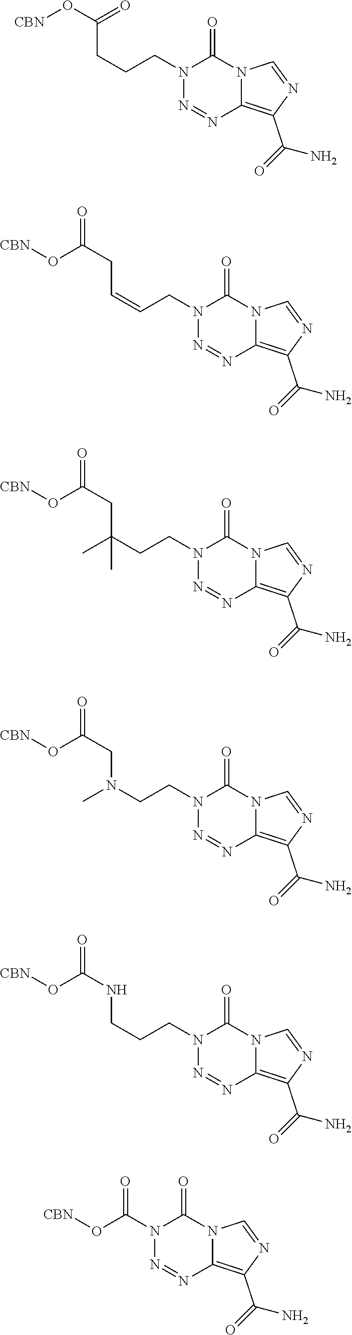

### Conjugate Molecules Comprising 5-Fluorouracil Analog Components

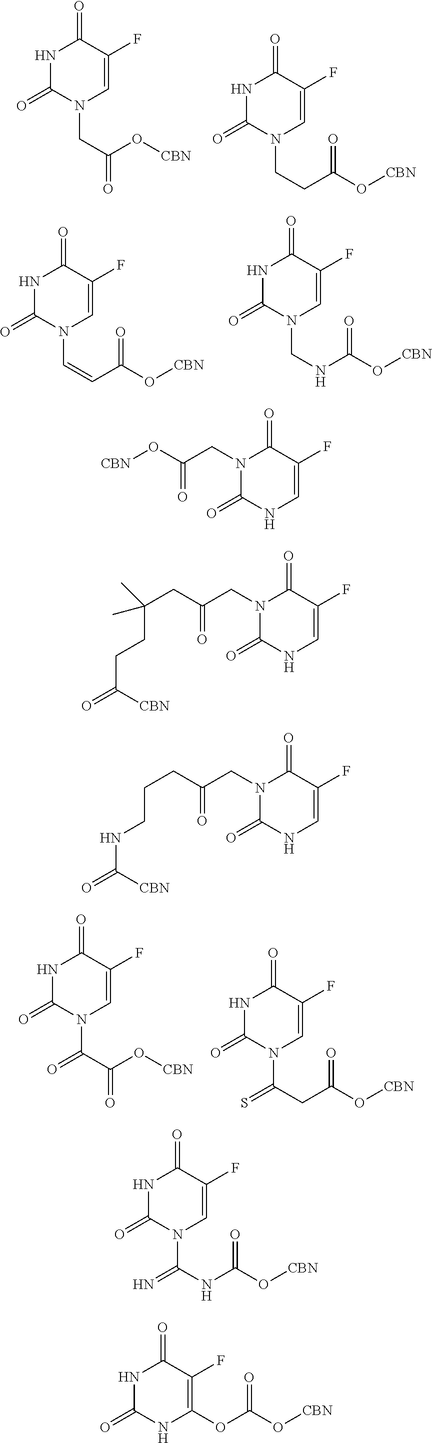

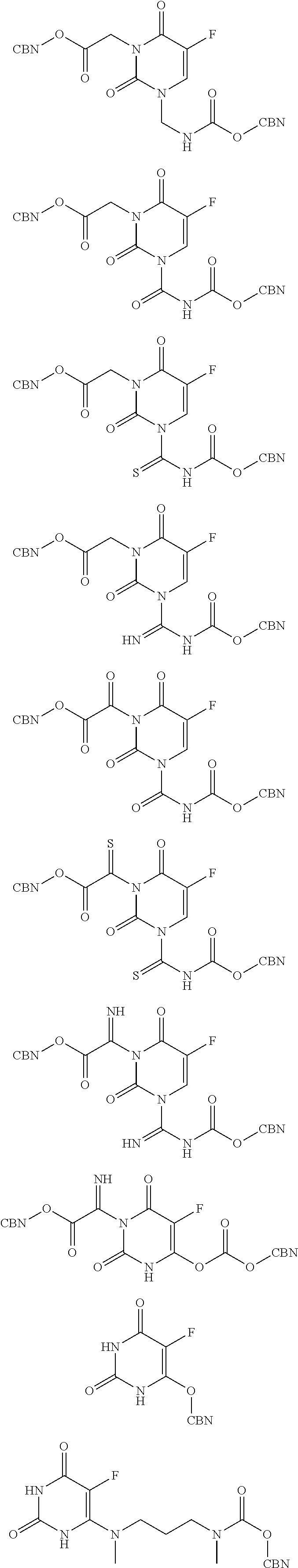

### Conjugate Molecules Comprising Diclofenac Components

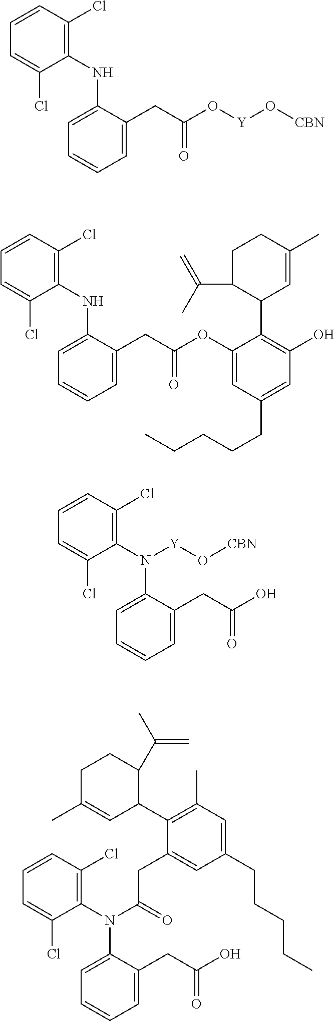

### Conjugate Molecules Comprising Celecoxib Components

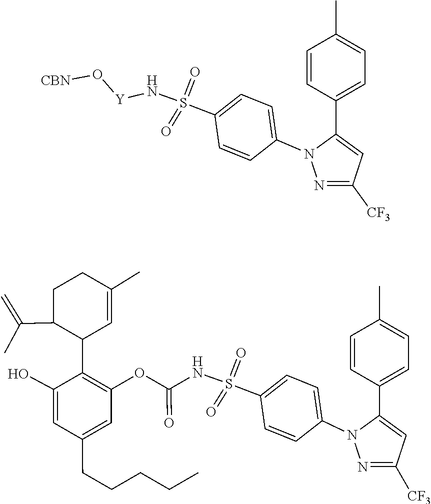

### Conjugate Molecules Comprising Gemcitabine Components

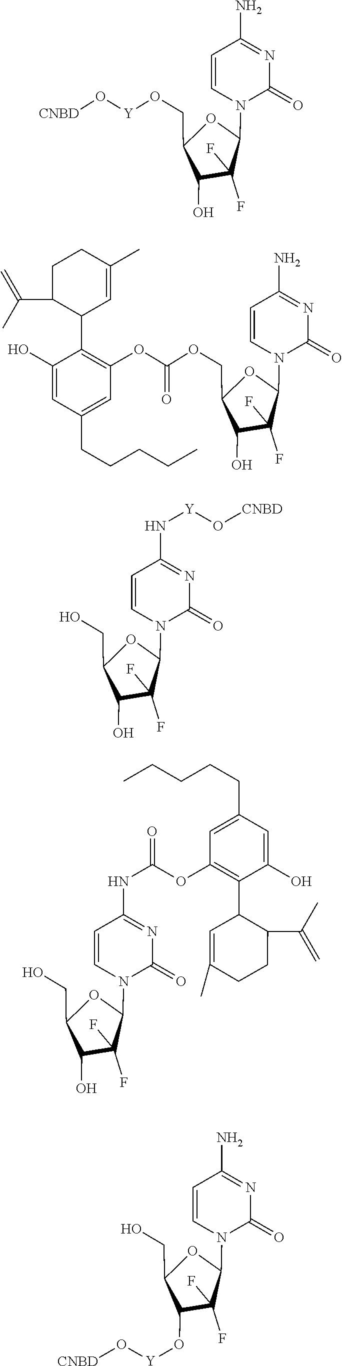

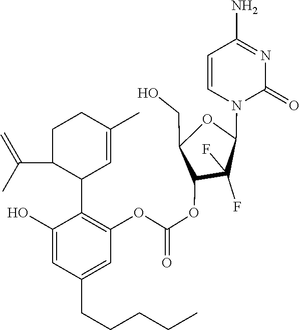

### Conjugate Molecules Comprising Axitinib Components

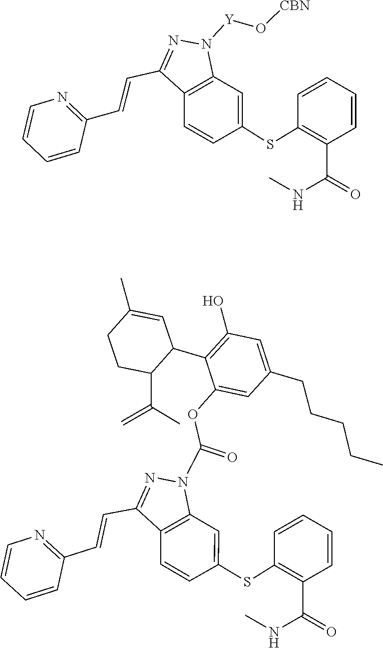

### Conjugate Molecules Comprising Batimastat Components

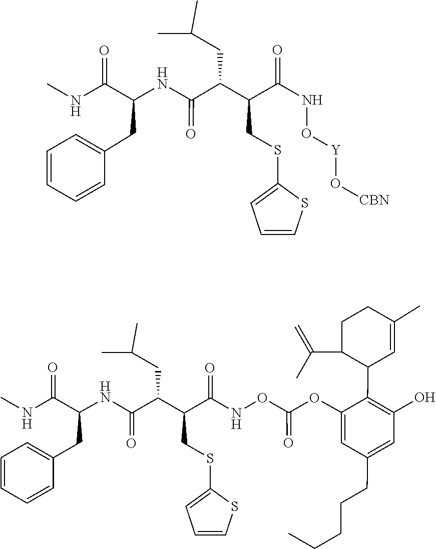

### Conjugate Molecules Comprising Bosutinib Components

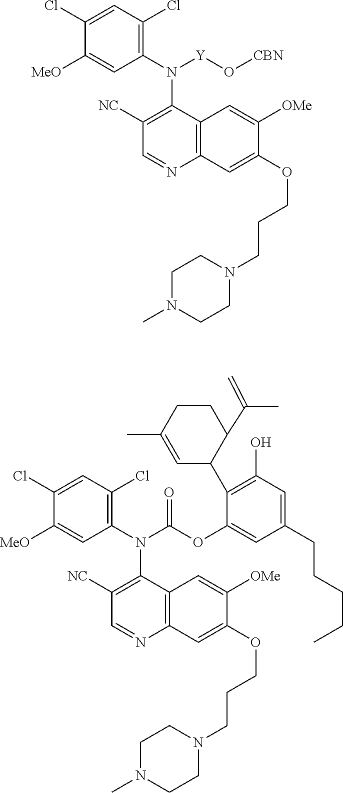

### Conjugate Molecules Comprising Crizotinib Components

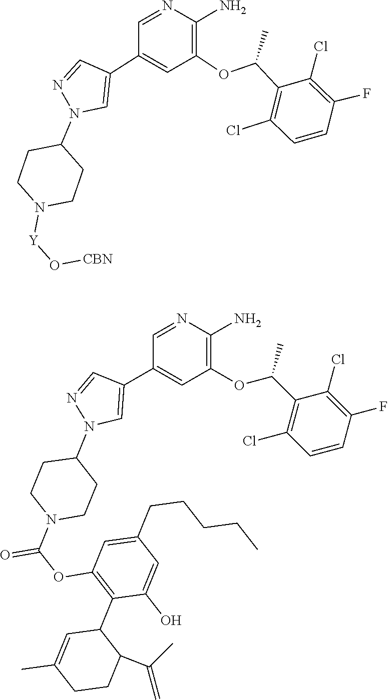

### Conjugate Molecules Comprising Erlotinib Components

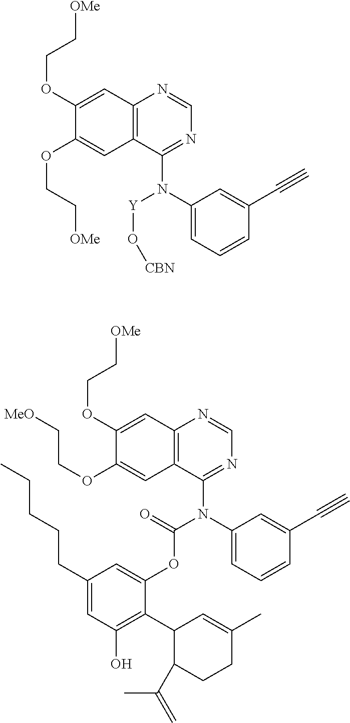

### Conjugate Molecules Comprising Everolimus Components

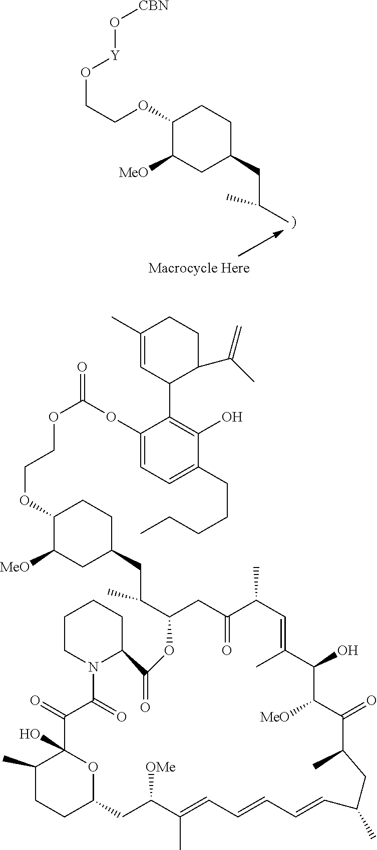

### Conjugate Molecules Comprising Ganetespib Components

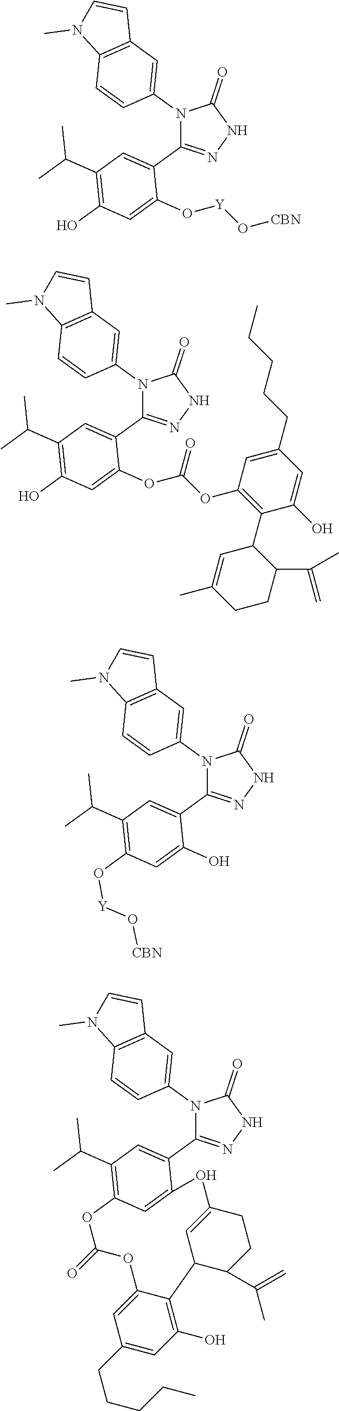

### Conjugate Molecules Comprising Glasdegib Components

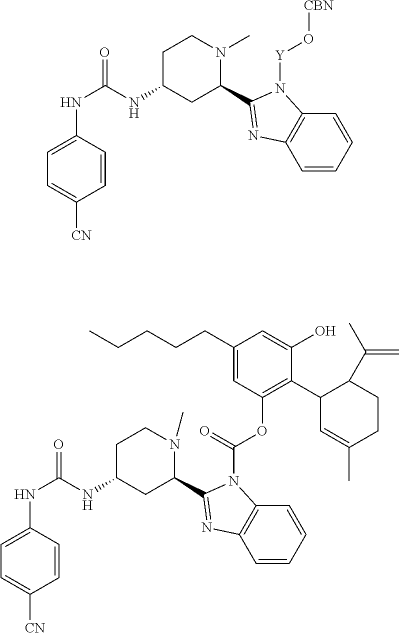

### Conjugate Molecules Comprising Imatinib Components

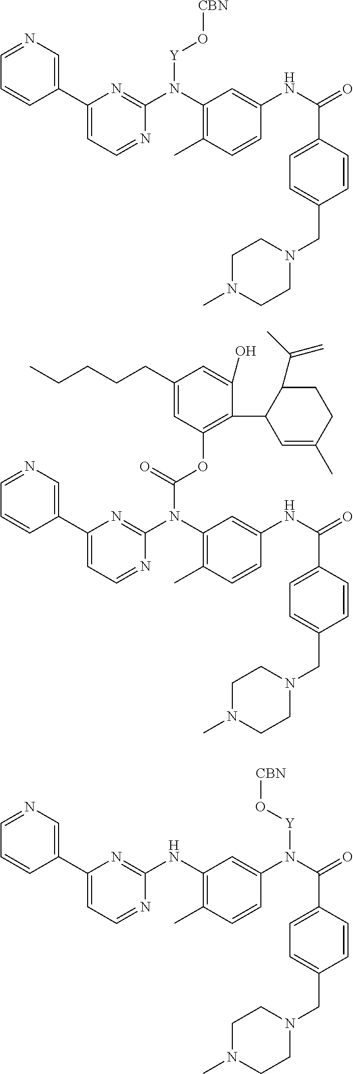

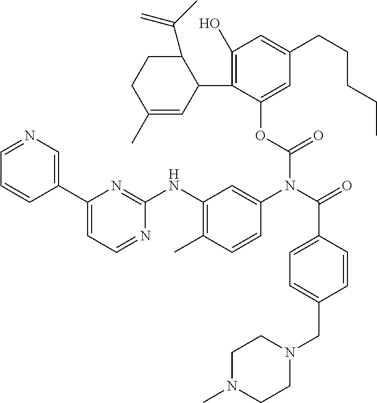 [SMILES: C=C(C)C1CCC(C)=CC1c1c(O)cc(CCCCC)cc1OC(=O)N(C(=O)c1ccc(CN2CCN(C)CC2)cc1)c1ccc(C)c(Nc2nccc(-c3cccnc3)n2)c1]

### Conjugate Molecules Comprising Lapatinib Components

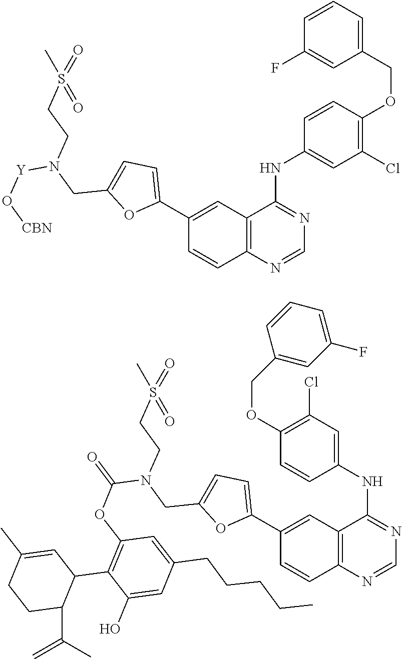

### Conjugate Molecules Comprising Navitoclax Components

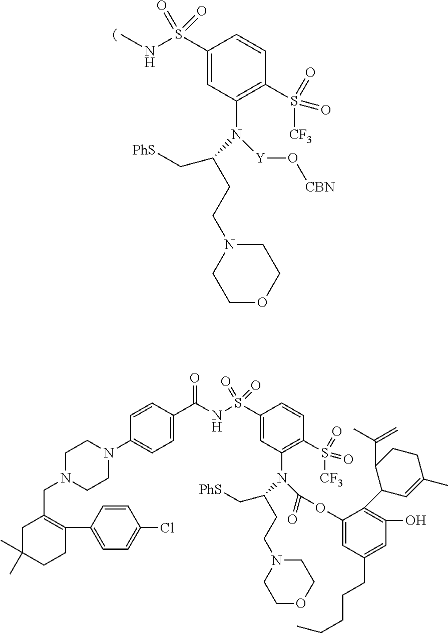

### Conjugate Molecules Comprising Nilotinib Components

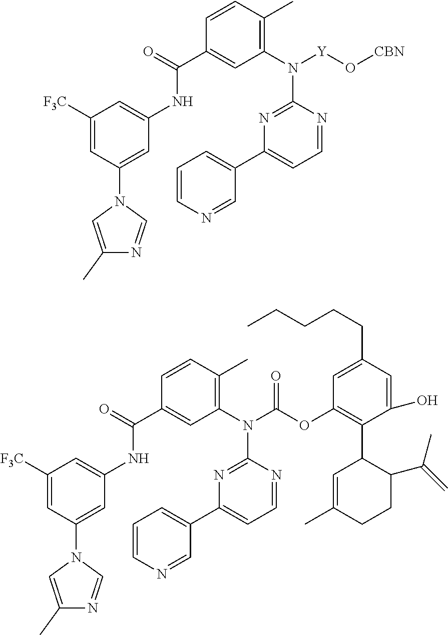

### Conjugate Molecules Comprising Luminespib (NVP-AUY922) Components

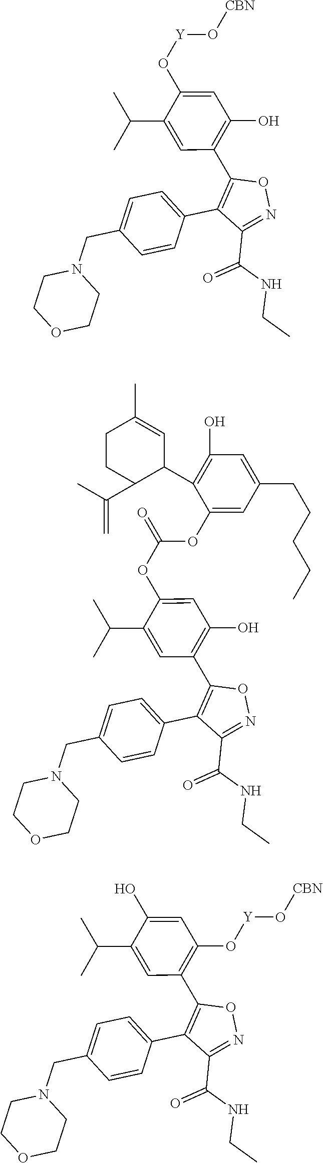

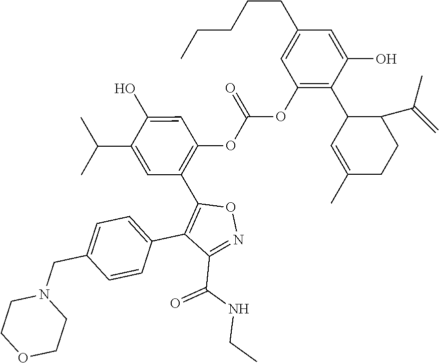 [SMILES: C=C(C)C1CCC(C)=CC1c1c(O)cc(CCCCC)cc1OC(=O)Oc1cc(O)c(C(C)C)cc1-c1onc(C(=O)NCC)c1-c1ccc(CN2CCOCC2)cc1]

### Conjugate Molecules Comprising Obatoclax Components

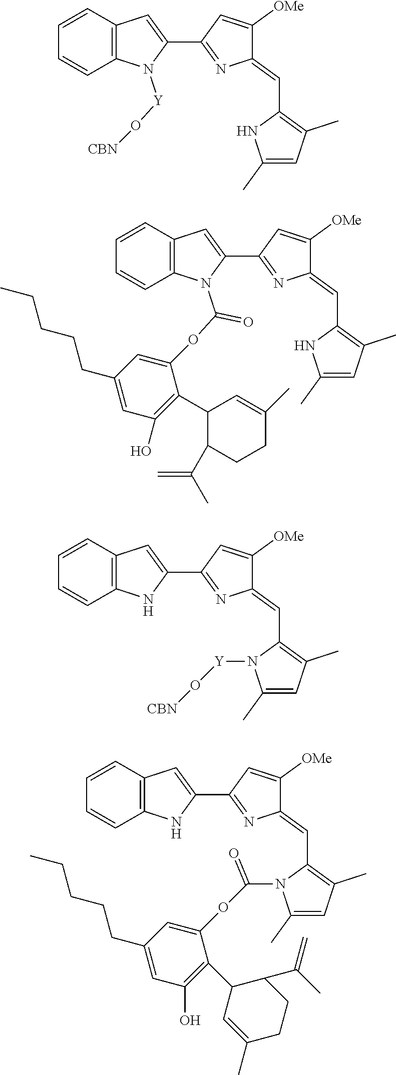

### Conjugate Molecules Comprising Ruxolitinib Components

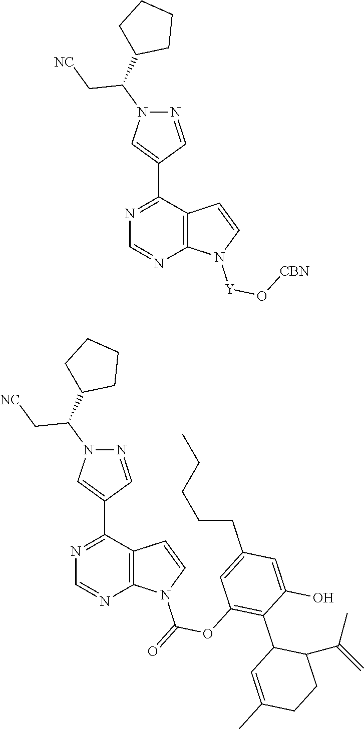

### Conjugate Molecules Comprising Saridegib Components

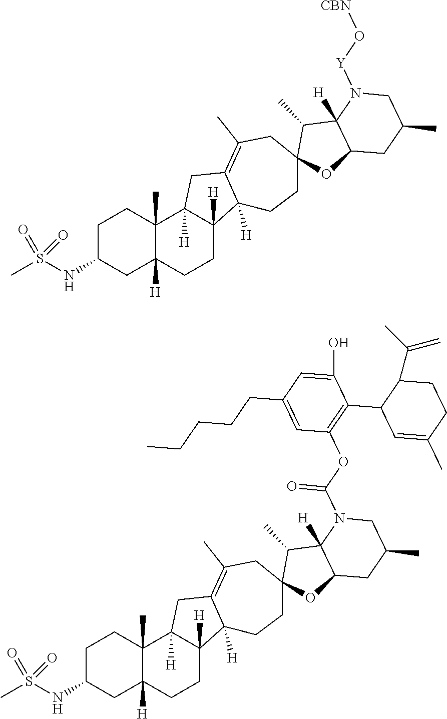

### Conjugate Molecules Comprising Sunitinib Components

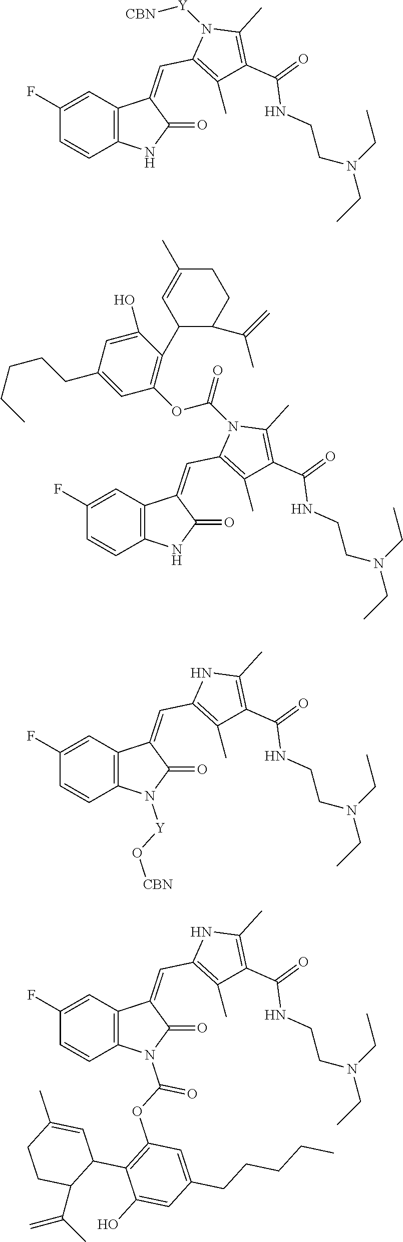

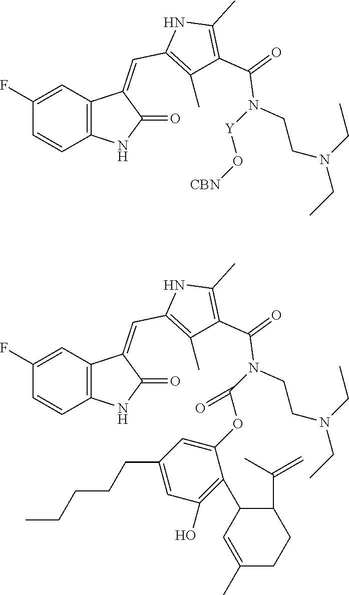

### Conjugate Molecules Comprising Trametinib Components

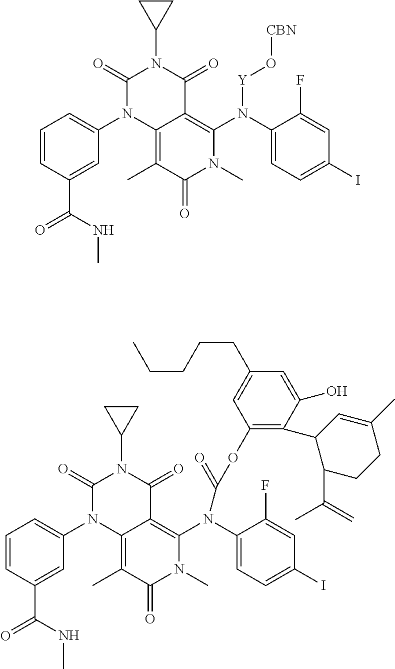

### Conjugate Molecules Comprising Warfarin Components

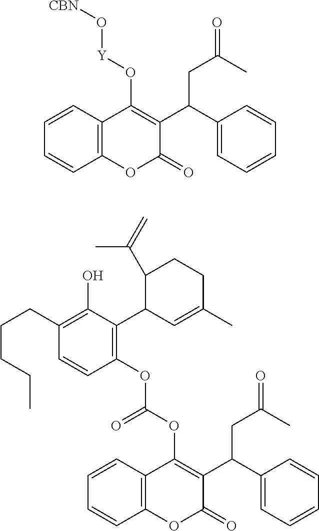

### Conjugate Molecules Comprising Daclatasvir Components

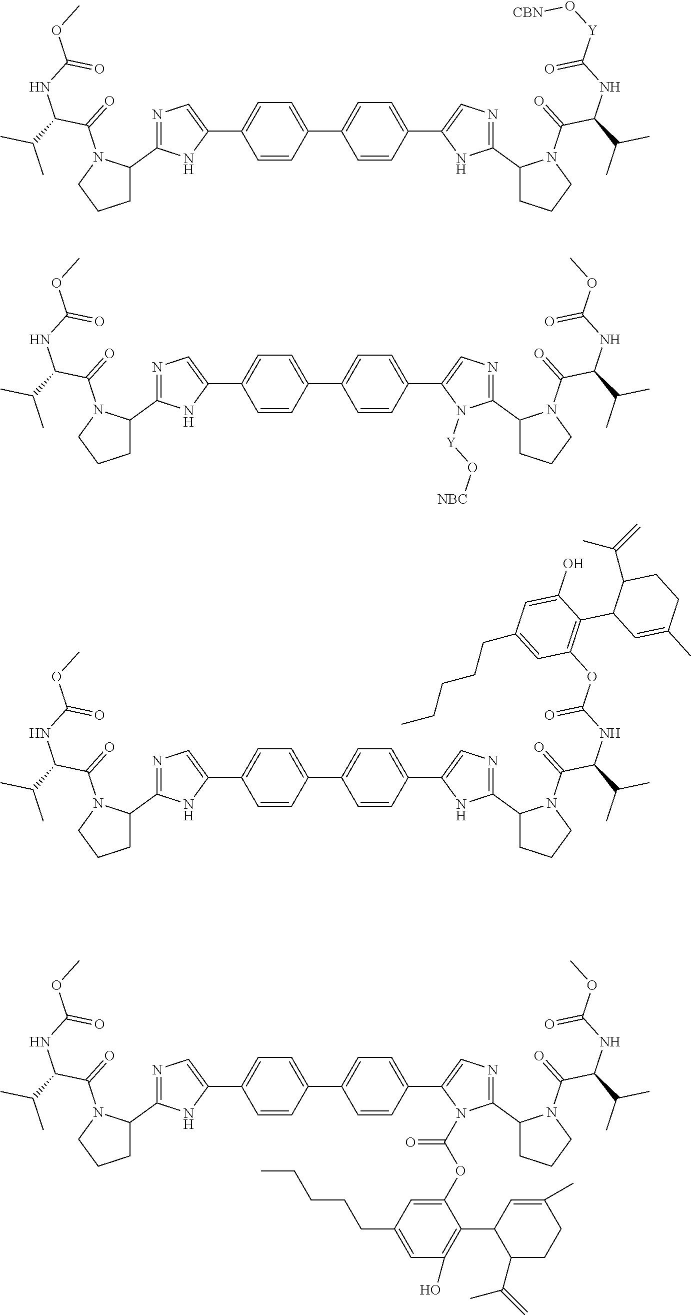

### Conjugate Molecules Comprising Etoposide Components

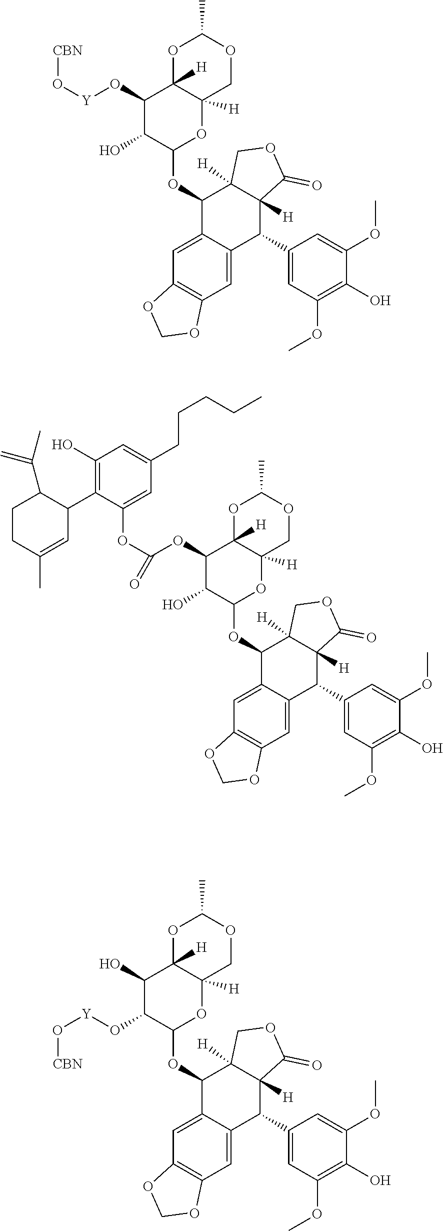

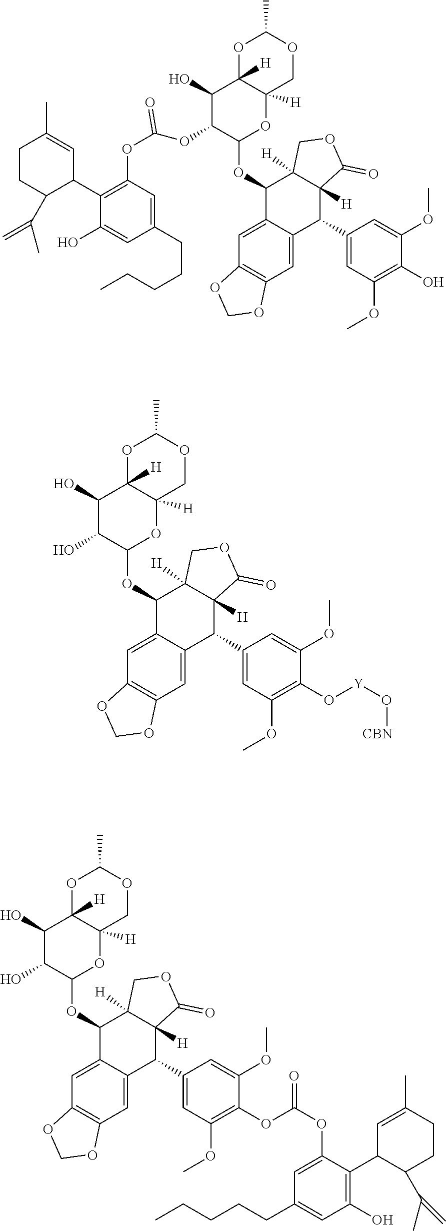

### Conjugate Molecules Comprising Atazanavir Components

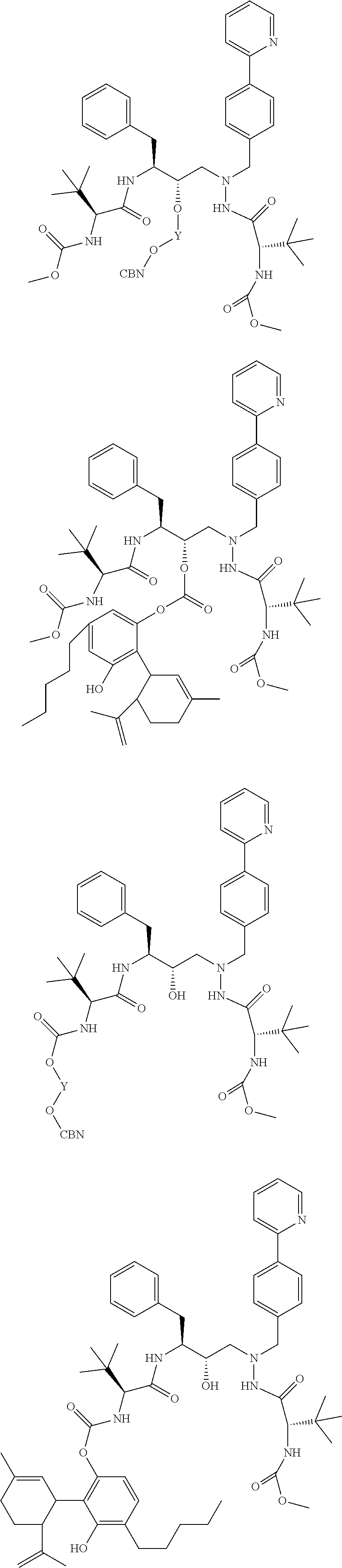

### Conjugate Molecules Comprising Pravastatin Components

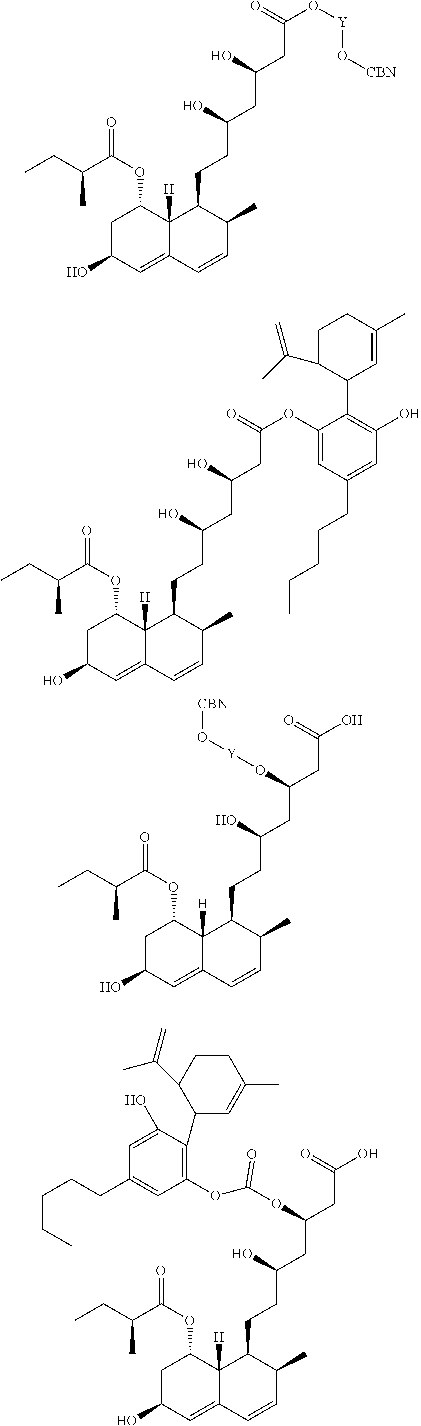

### Conjugate Molecules Comprising Dasatinib Components

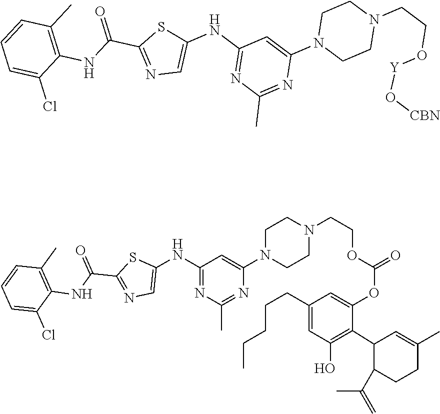

### Conjugate Molecules Comprising Didanosine Components

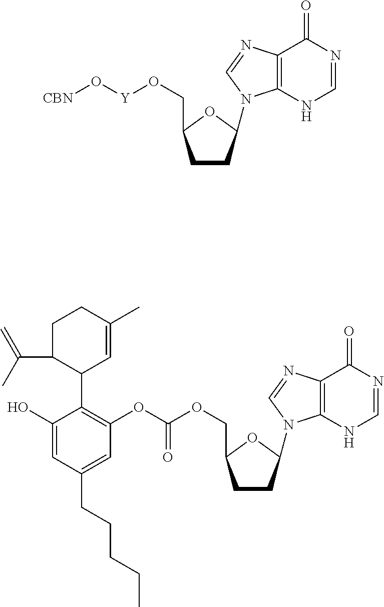

conjugate molecules comprising stavudine components

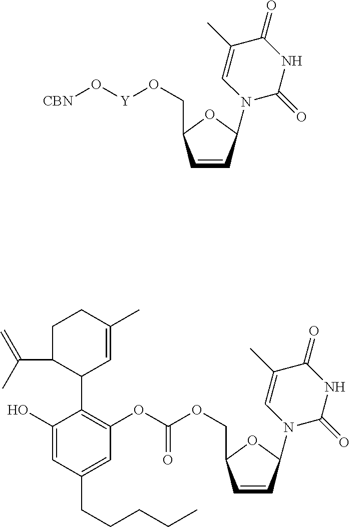

### Examples of Conjugate Molecules Containing Epoxide, Aziridine, Sulfonate, or Halide Components

Examples of conjugate molecules containing epoxide, aziridine, sulfonate, or halide components using a variety of linker types are shown below. For simplicity, the cannabinoid component is a cannabidiol component linked to a single therapeutic agent moiety. “X” in some of the examples represents a halide (Cl, Br, or I).

Carbamate Linkers

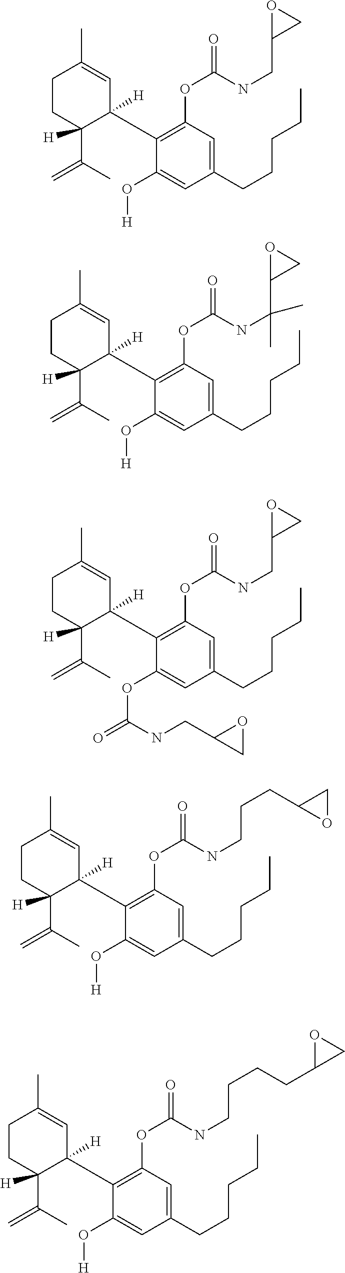

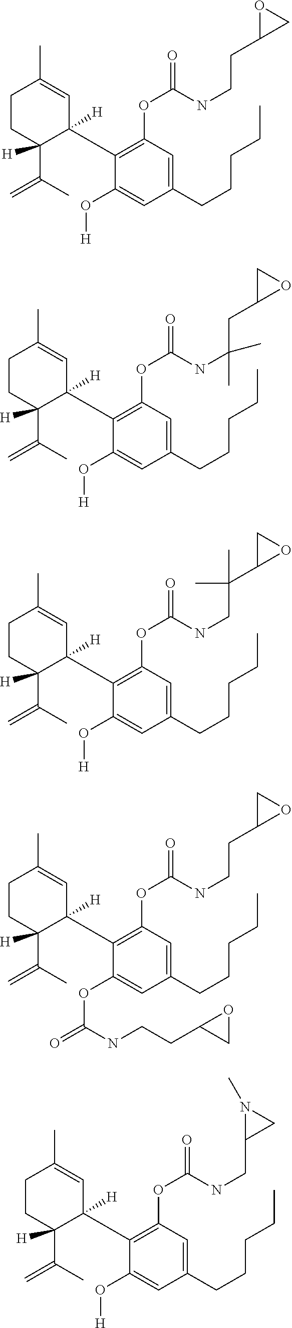

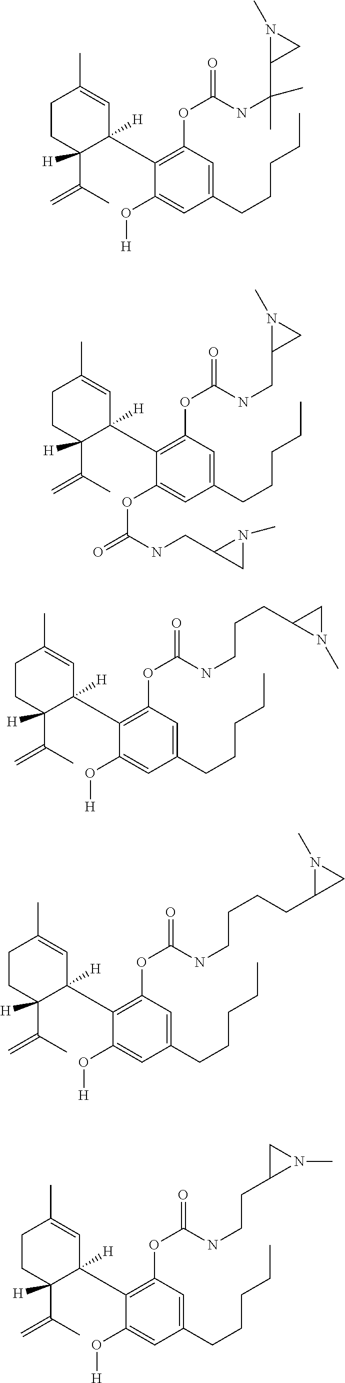

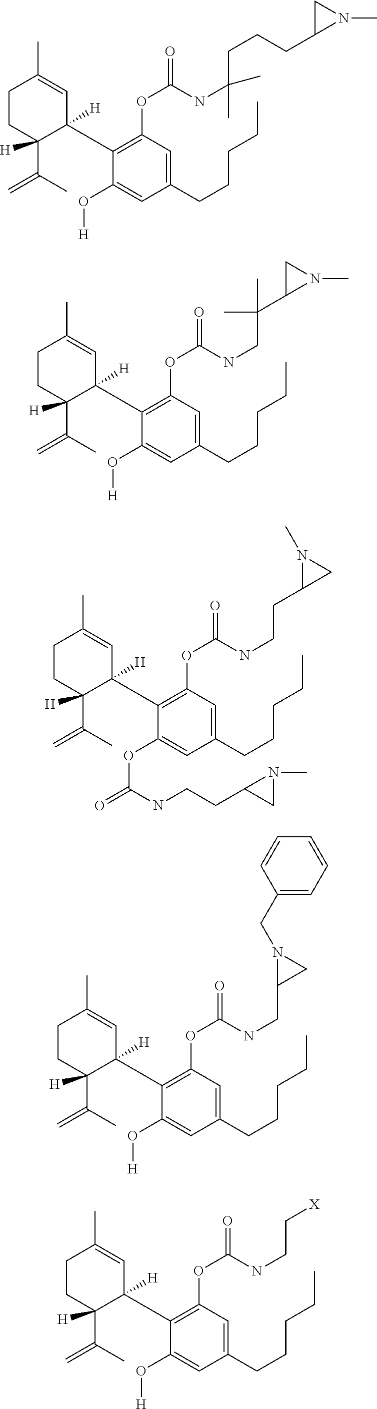

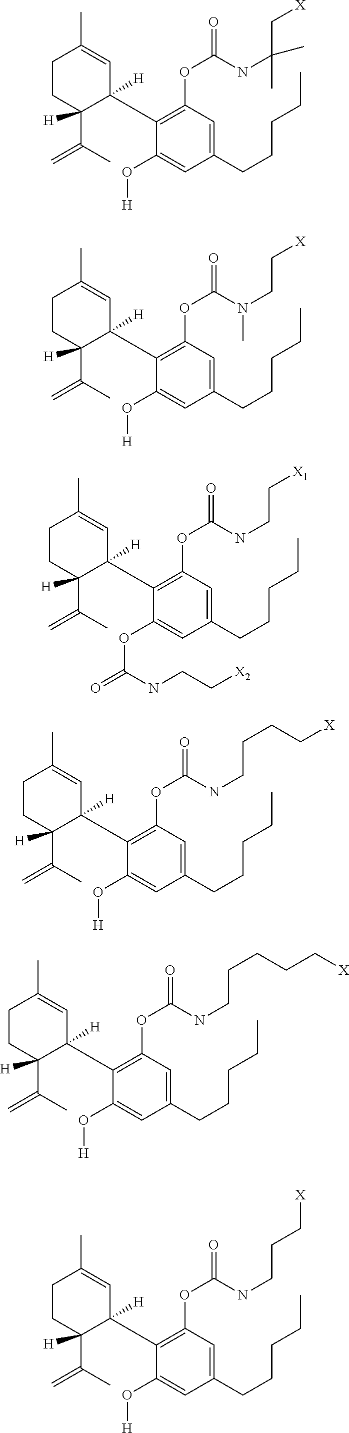

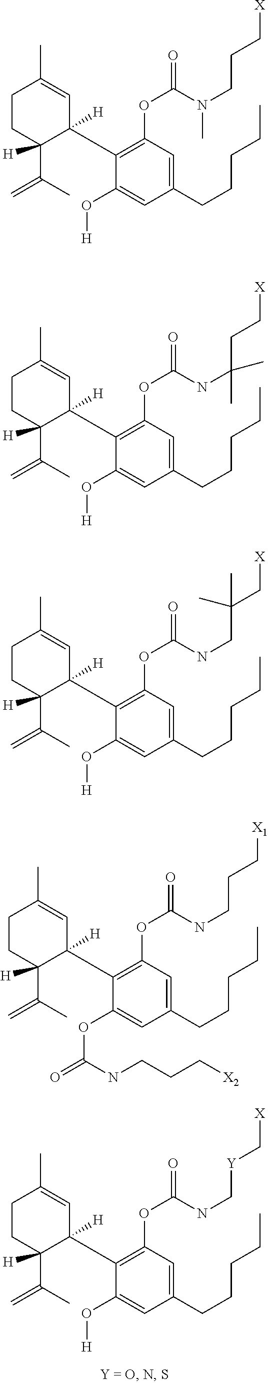

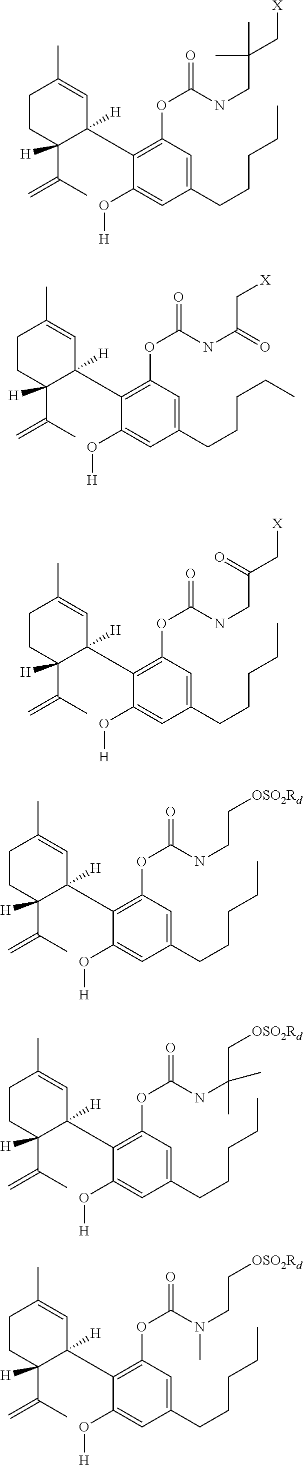

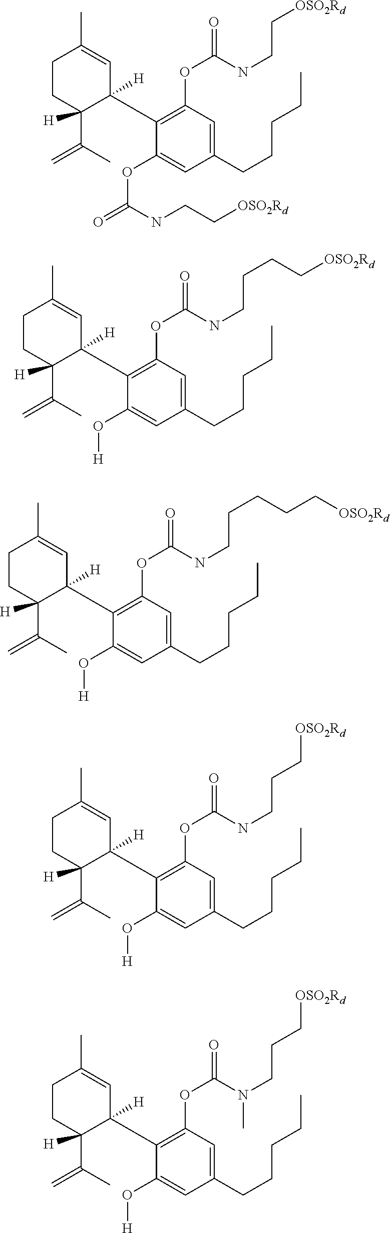

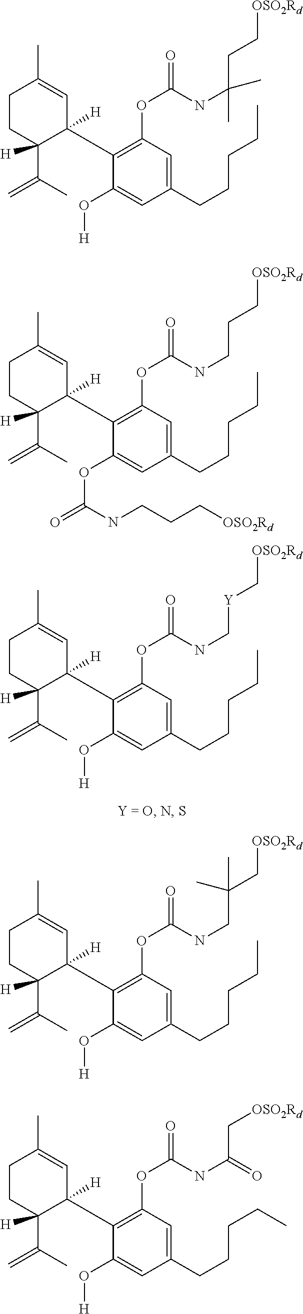

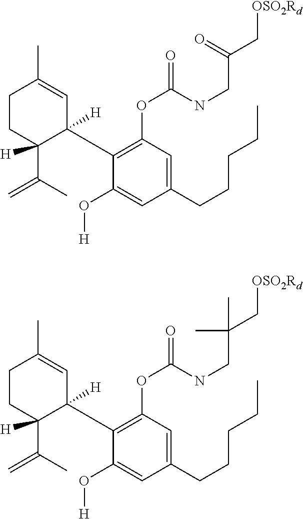

### Carbonate Linkers

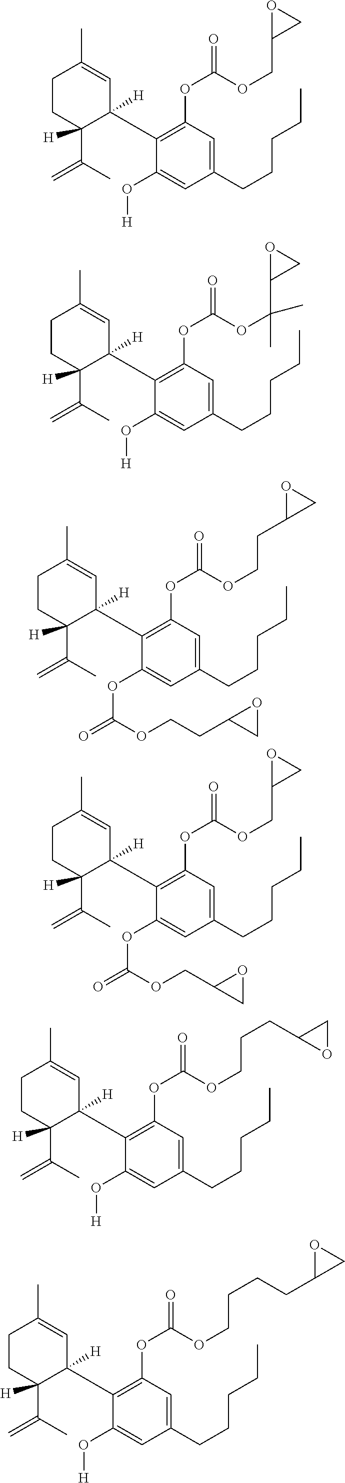

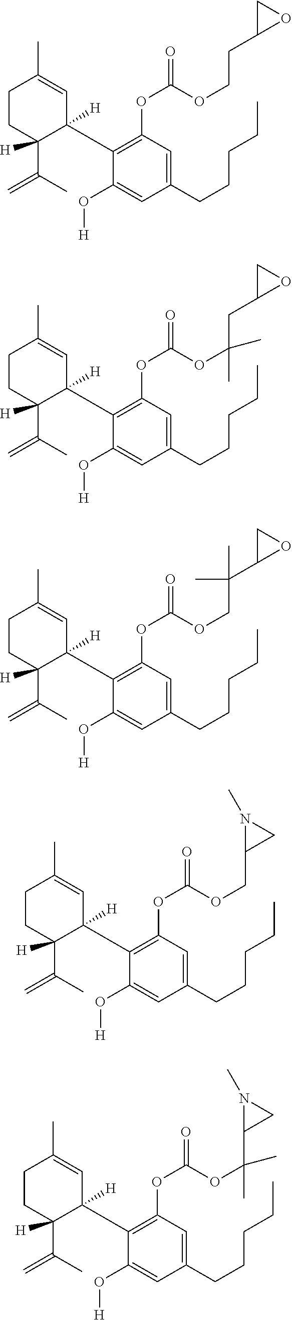 [SMILES: C=C(C)[C@@H]1CCC(C)=C[C@H]1c1c(O)cc(CCCCC)cc1OC(=O)OCCC1CO1 ; C=C(C)[C@@H]1CCC(C)=C[C@H]1c1c(O)cc(CCCCC)cc1OC(=O)OC(C)(C)CC1CO1 ; C=C(C)[C@@H]1CCC(C)=C[C@H]1c1c(O)cc(CCCCC)cc1OC(=O)OCC(C)(C)C1CO1 ; C=C(C)[C@@H]1CCC(C)=C[C@H]1c1c(O)cc(CCCCC)cc1OC(=O)OCC1CN1C ; C=C(C)[C@@H]1CCC(C)=C[C@H]1c1c(O)cc(CCCCC)cc1OC(=O)OC(C)(C)C1CN1C]

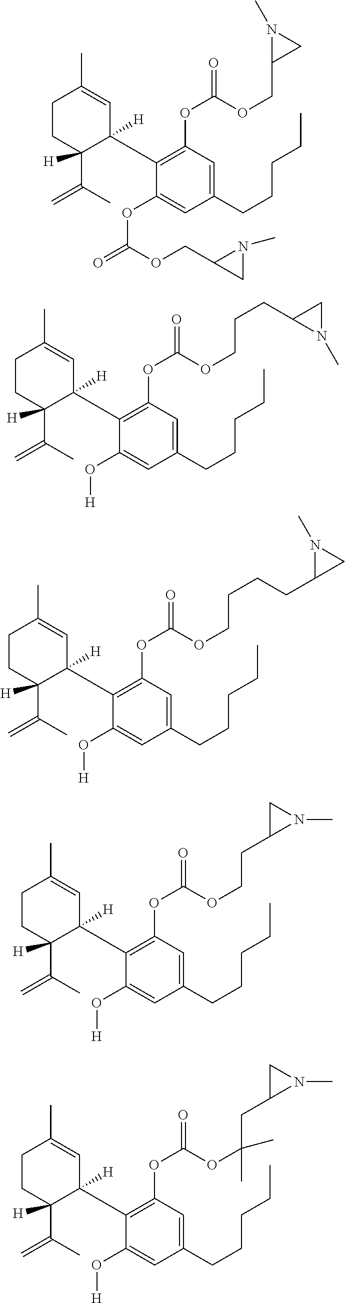

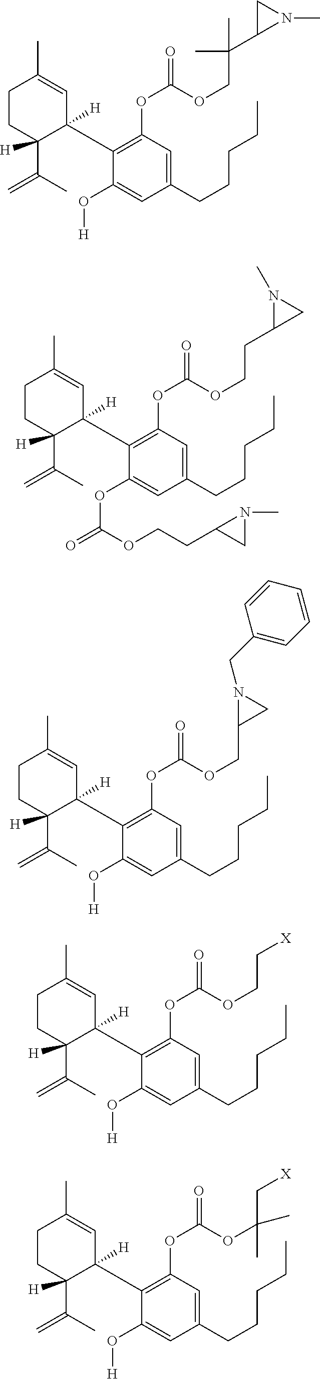

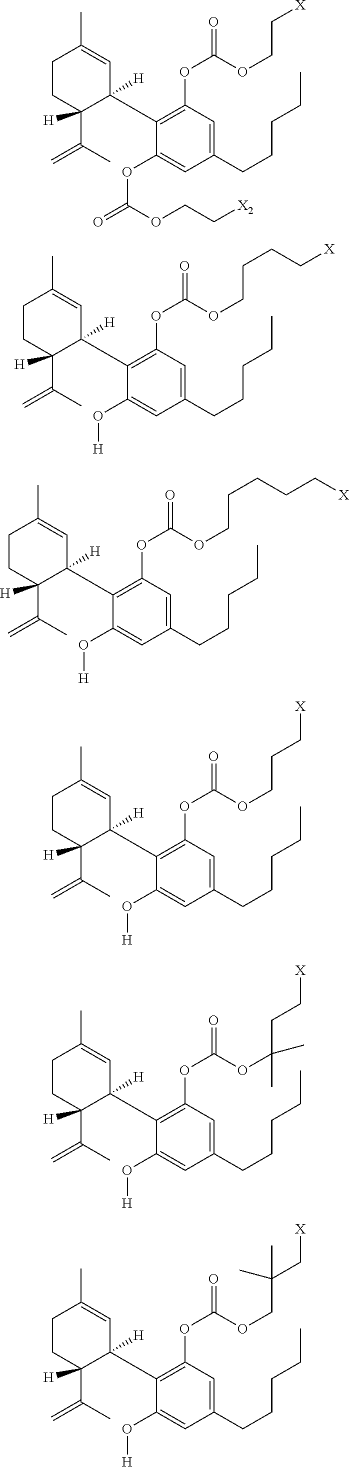

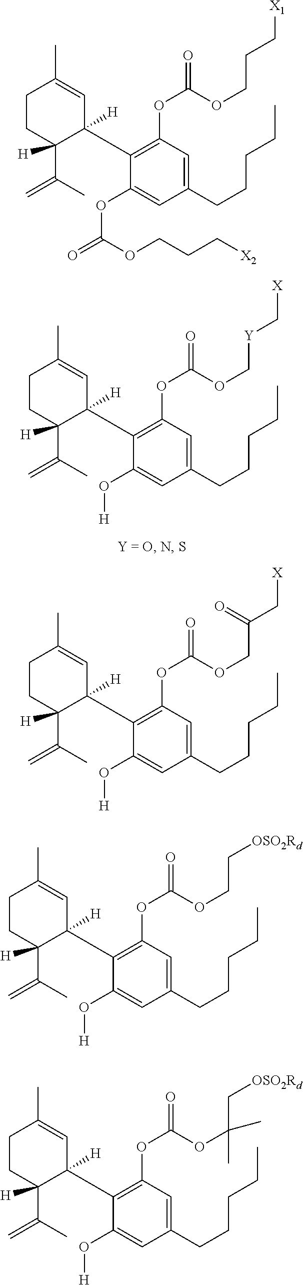

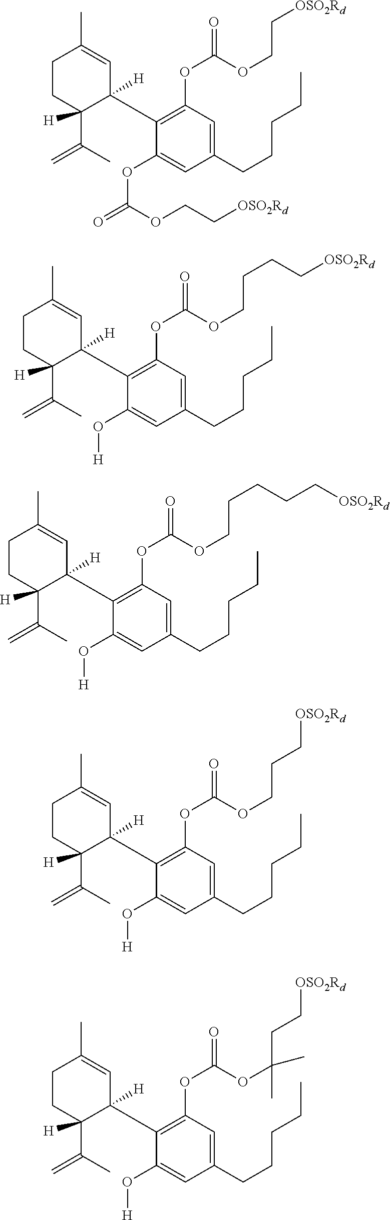

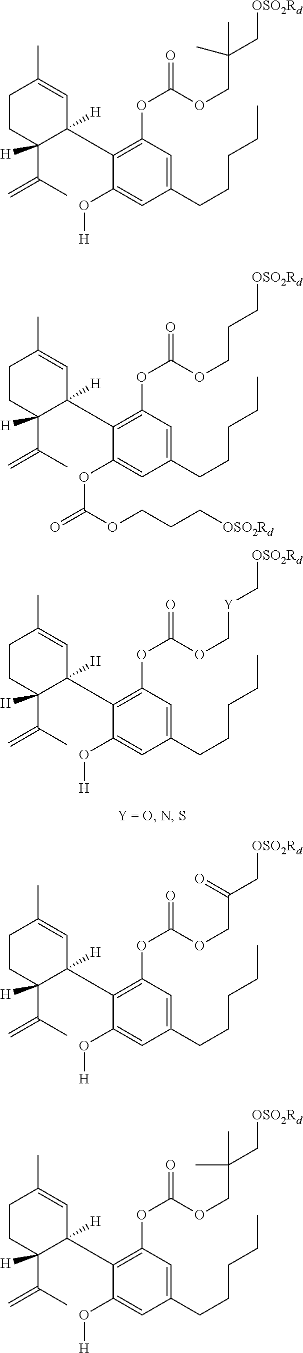

### Xanthate Linkers

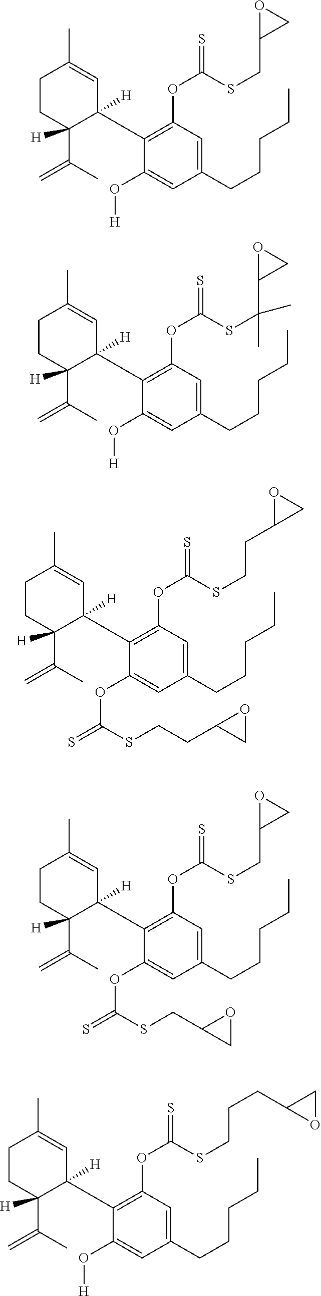 [SMILES: C=C(C)[C@@H]1CCC(C)=C[C@H]1c1c(O)cc(CCCCC)cc1OC(=S)SCC1CO1 ; C=C(C)[C@@H]1CCC(C)=C[C@H]1c1c(O)cc(CCCCC)cc1OC(=S)SC(C)(C)C1CO1 ; C=C(C)[C@@H]1CCC(C)=C[C@H]1c1c(OC(=S)SCCC2CO2)cc(CCCCC)cc1OC(=S)SCCC1CO1 ; C=C(C)[C@@H]1CCC(C)=C[C@H]1c1c(OC(=S)SCC2CO2)cc(CCCCC)cc1OC(=S)SCC1CO1 ; C=C(C)[C@@H]1CCC(C)=C[C@H]1c1c(O)cc(CCCCC)cc1OC(=S)SCCCC1CO1]

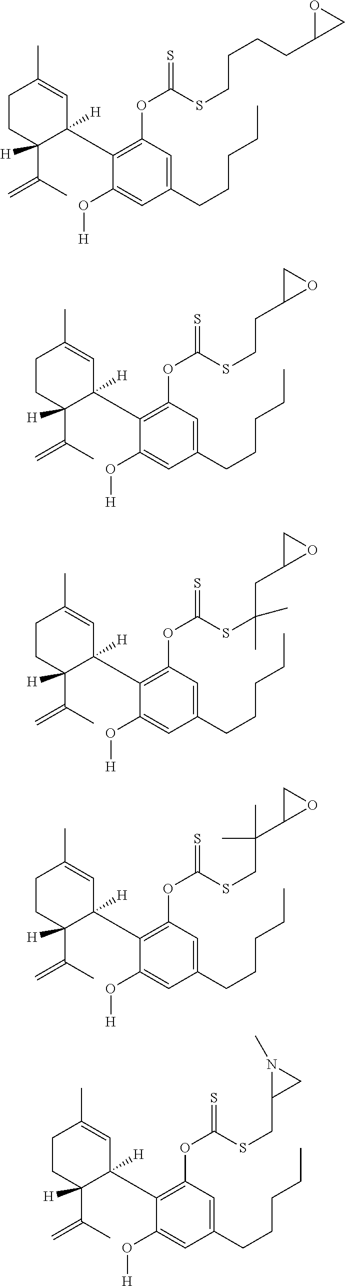 [SMILES: C=C(C)[C@@H]1CCC(C)=C[C@H]1c1c(O)cc(CCCCC)cc1OC(=S)SCCCCC1CO1 ; C=C(C)[C@@H]1CCC(C)=C[C@H]1c1c(O)cc(CCCCC)cc1OC(=S)SCCC1CO1 ; C=C(C)[C@@H]1CCC(C)=C[C@H]1c1c(O)cc(CCCCC)cc1OC(=S)SC(C)(C)CC1CO1 ; C=C(C)[C@@H]1CCC(C)=C[C@H]1c1c(O)cc(CCCCC)cc1OC(=S)SCC(C)(C)C1CO1 ; C=C(C)[C@@H]1CCC(C)=C[C@H]1c1c(O)cc(CCCCC)cc1OC(=S)SCC1CN1C]

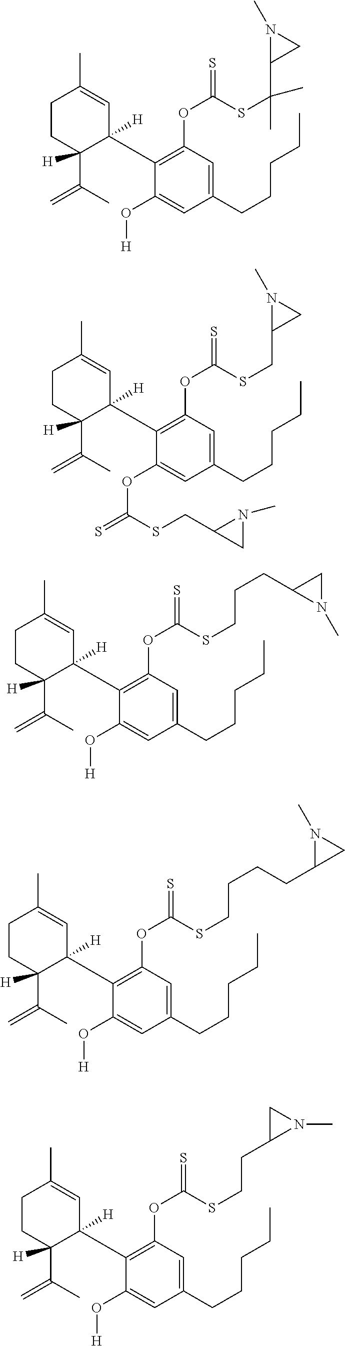

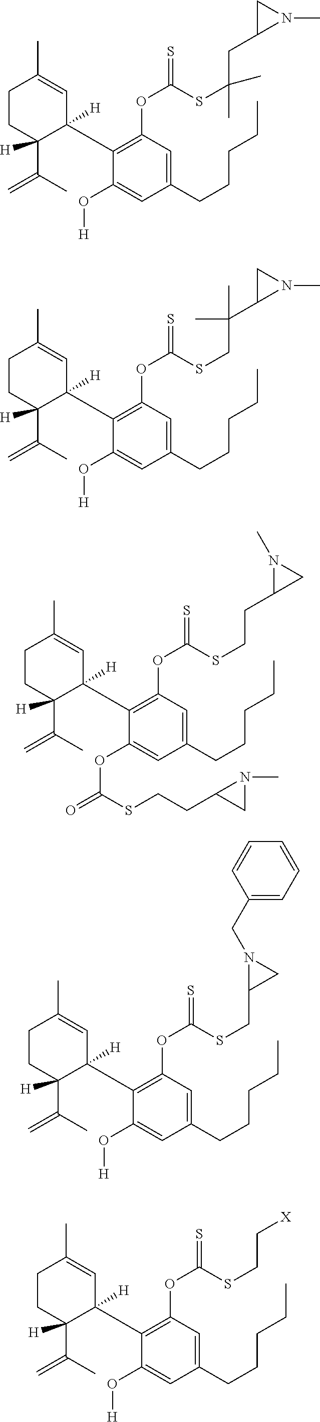

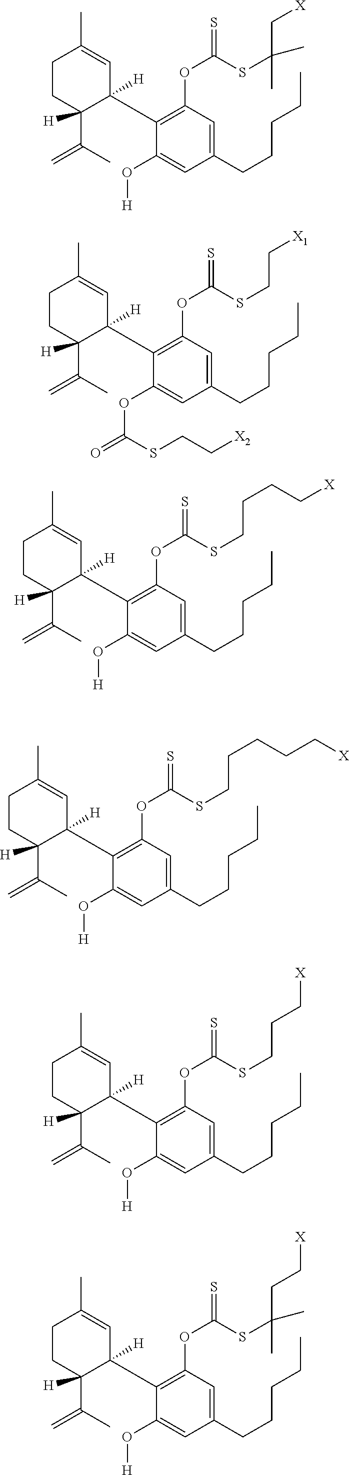

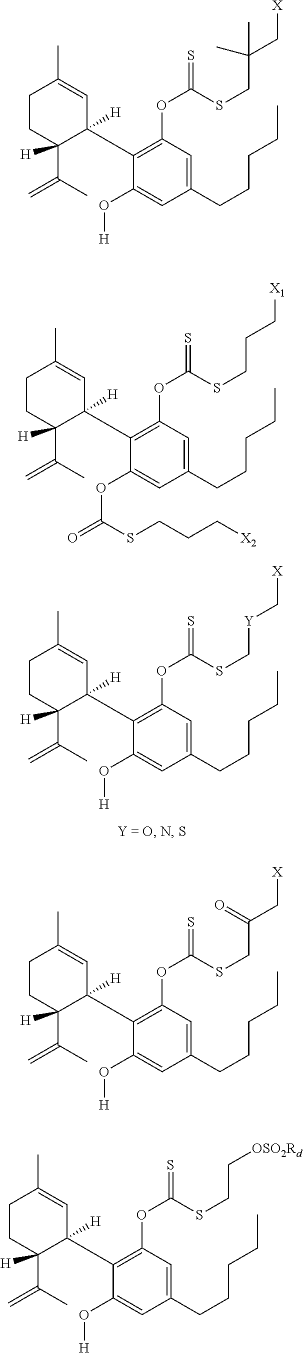

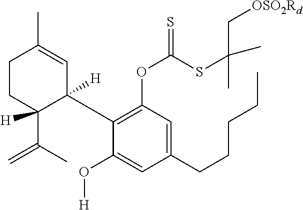

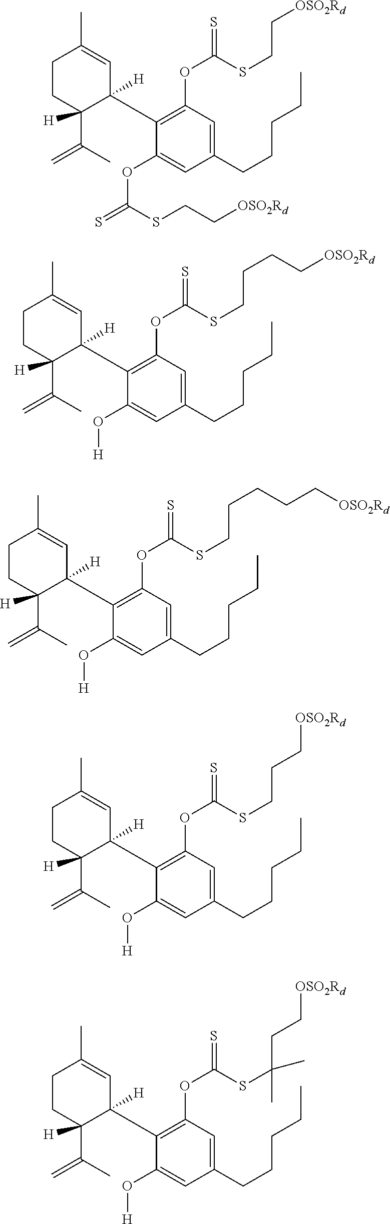

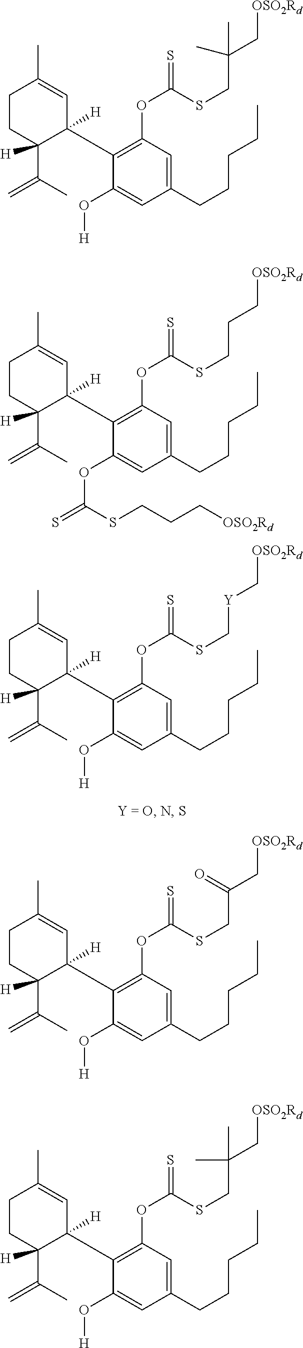

### Ester Linkers

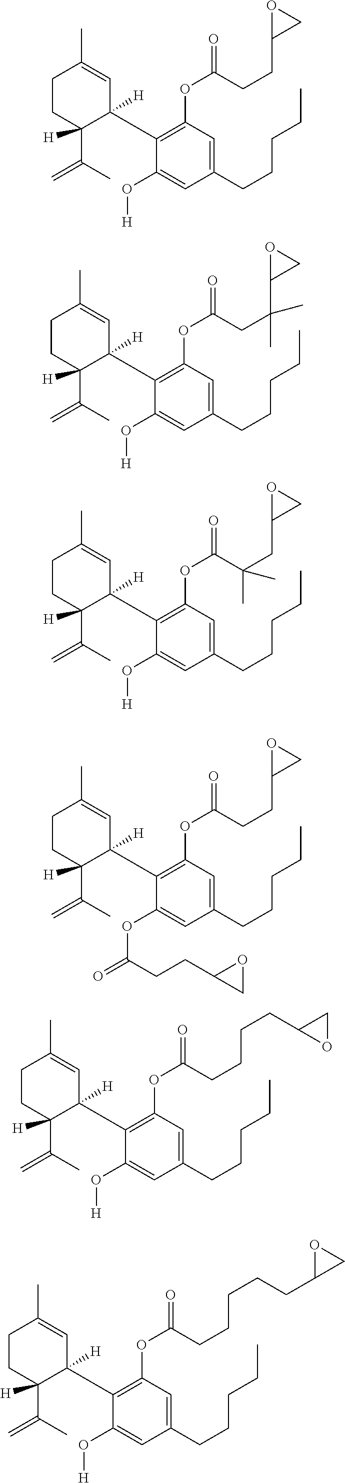

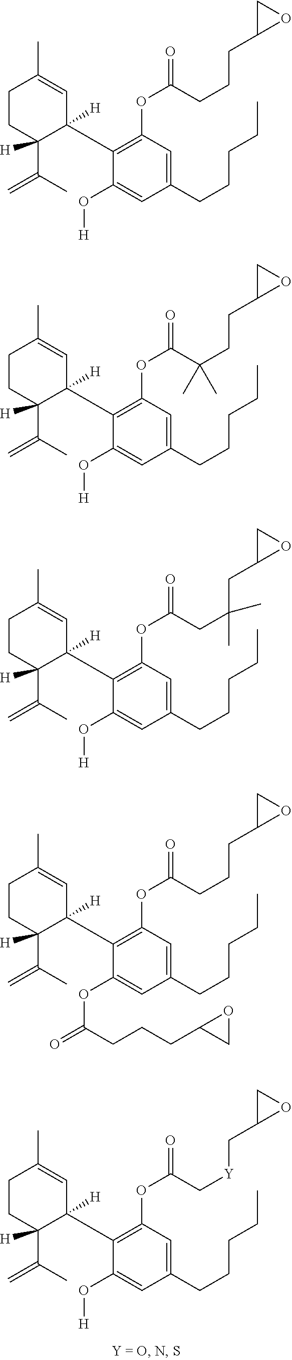

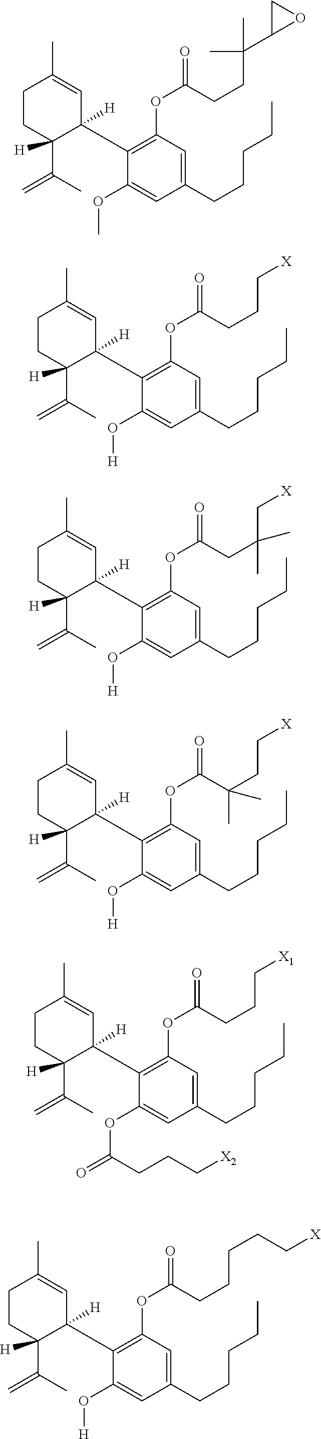

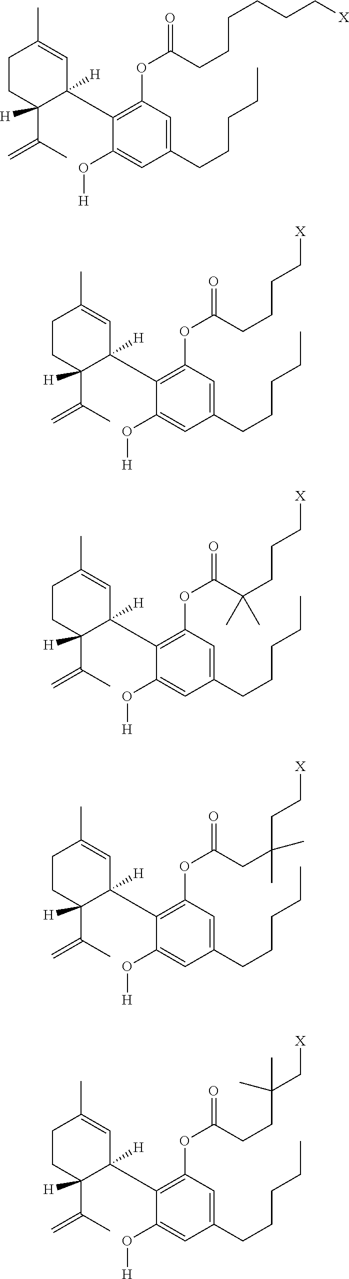

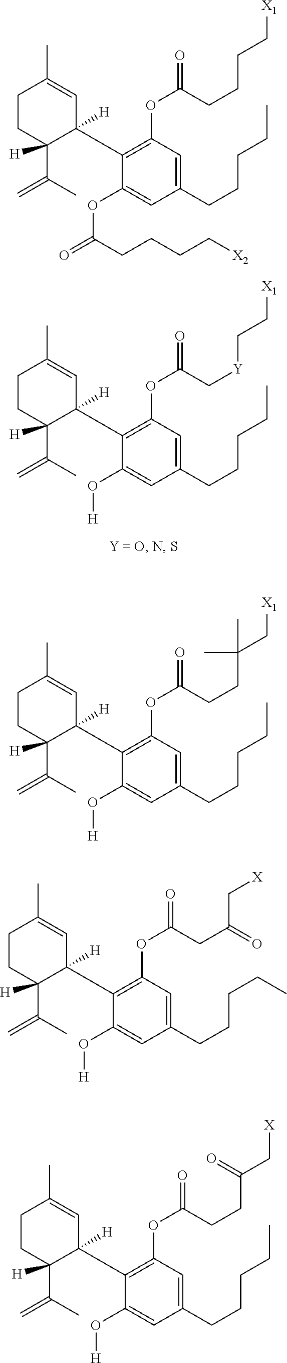

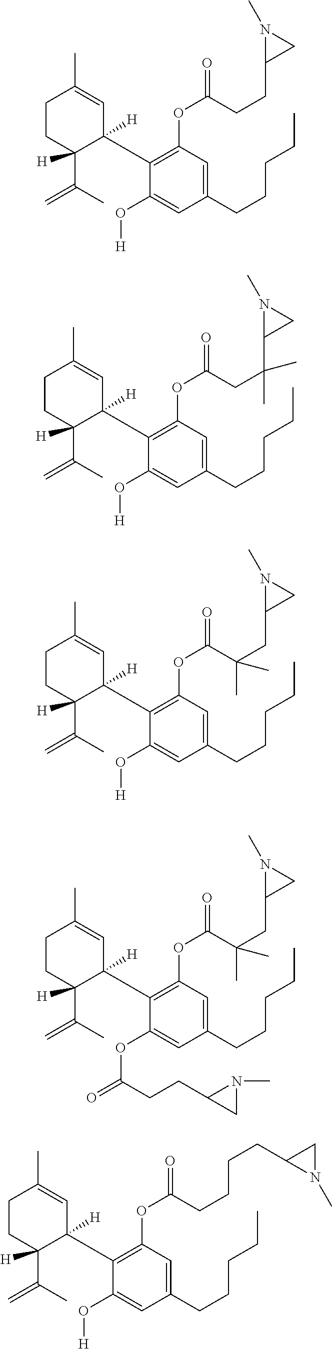

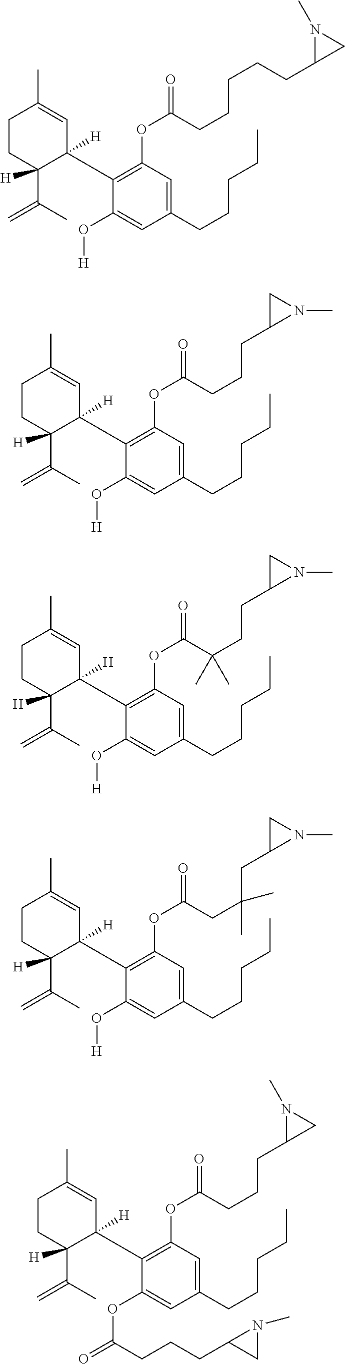 [SMILES: C=C(C)[C@@H]1CCC(C)=C[C@H]1c1c(O)cc(CCCCC)cc1OC(=O)CCCCCC1CN1C ; C=C(C)[C@@H]1CCC(C)=C[C@H]1c1c(O)cc(CCCCC)cc1OC(=O)CCCC1CN1C ; C=C(C)[C@@H]1CCC(C)=C[C@H]1c1c(O)cc(CCCCC)cc1OC(=O)C(C)(C)CCC1CN1C ; C=C(C)[C@@H]1CCC(C)=C[C@H]1c1c(O)cc(CCCCC)cc1OC(=O)CC(C)(C)CC1CN1C ; C=C(C)[C@@H]1CCC(C)=C[C@H]1c1c(OC(=O)CCCC2CN2C)cc(CCCCC)cc1OC(=O)CCCC1CN1C]

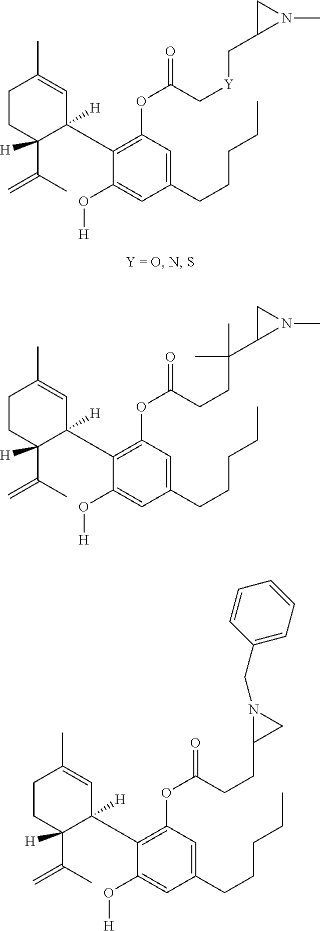

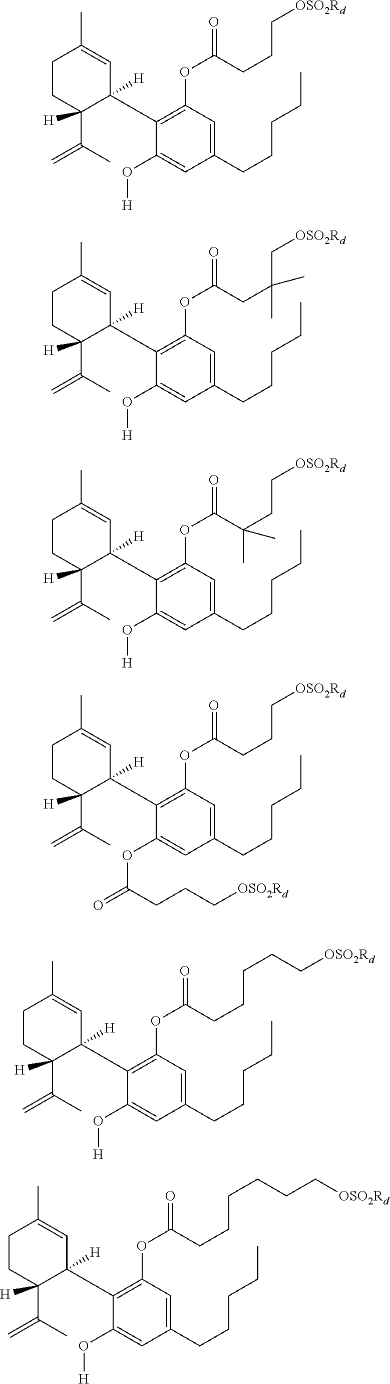

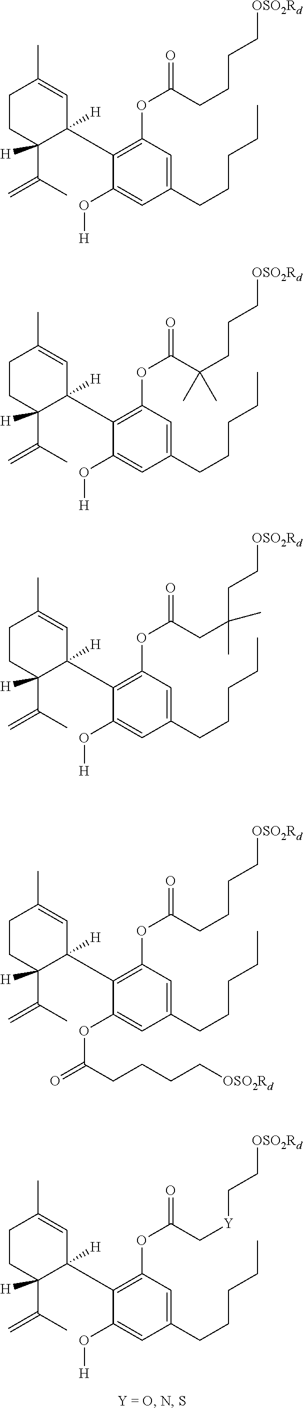

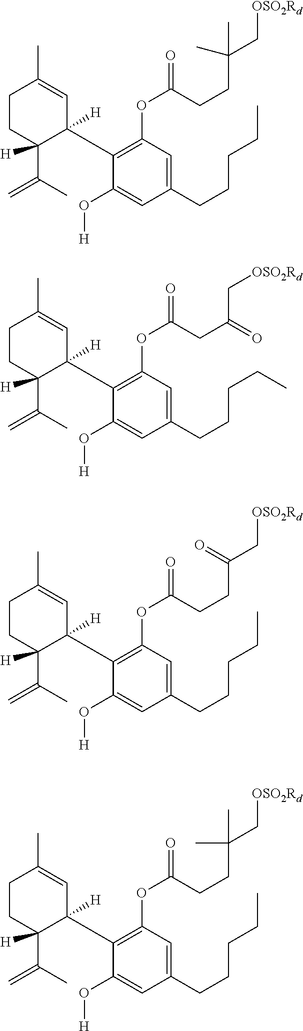

### Thiocarbamate Linkers

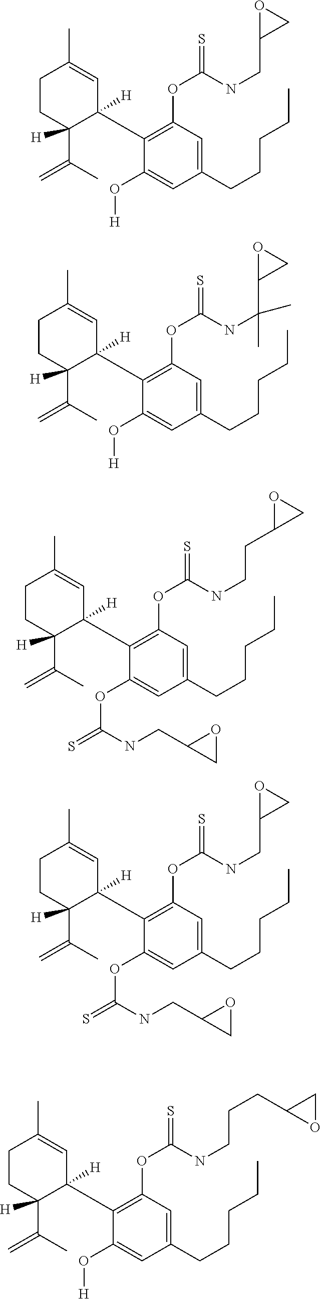 [SMILES: C=C(C)[C@@H]1CCC(C)=C[C@H]1c1c(O)cc(CCCCC)cc1OC(=S)NCC1CO1 ; C=C(C)[C@@H]1CCC(C)=C[C@H]1c1c(O)cc(CCCCC)cc1OC(=S)NC(C)(C)C1CO1 ; C=C(C)[C@@H]1CCC(C)=C[C@H]1c1c(OC(=S)NCCC2CO2)cc(CCCCC)cc1OC(=S)NCC1CO1 ; C=C(C)[C@@H]1CCC(C)=C[C@H]1c1c(OC(=S)NCC2CO2)cc(CCCCC)cc1OC(=S)NCC1CO1 ; C=C(C)[C@@H]1CCC(C)=C[C@H]1c1c(O)cc(CCCCC)cc1OC(=S)NCCCC1CO1]

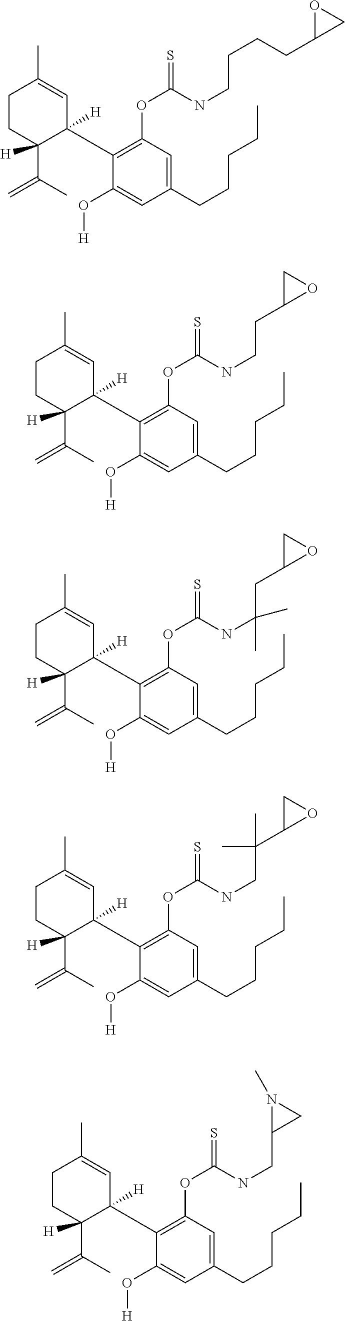 [SMILES: C=C(C)[C@@H]1CCC(C)=C[C@H]1c1c(O)cc(CCCCC)cc1OC(=S)NCCCCC1CO1 ; C=C(C)[C@@H]1CCC(C)=C[C@H]1c1c(O)cc(CCCCC)cc1OC(=S)NCCC1CO1 ; C=C(C)[C@@H]1CCC(C)=C[C@H]1c1c(O)cc(CCCCC)cc1OC(=S)NC(C)(C)CC1CO1 ; C=C(C)[C@@H]1CCC(C)=C[C@H]1c1c(O)cc(CCCCC)cc1OC(=S)NCC(C)(C)C1CO1 ; C=C(C)[C@@H]1CCC(C)=C[C@H]1c1c(O)cc(CCCCC)cc1OC(=S)NCC1CN1C]

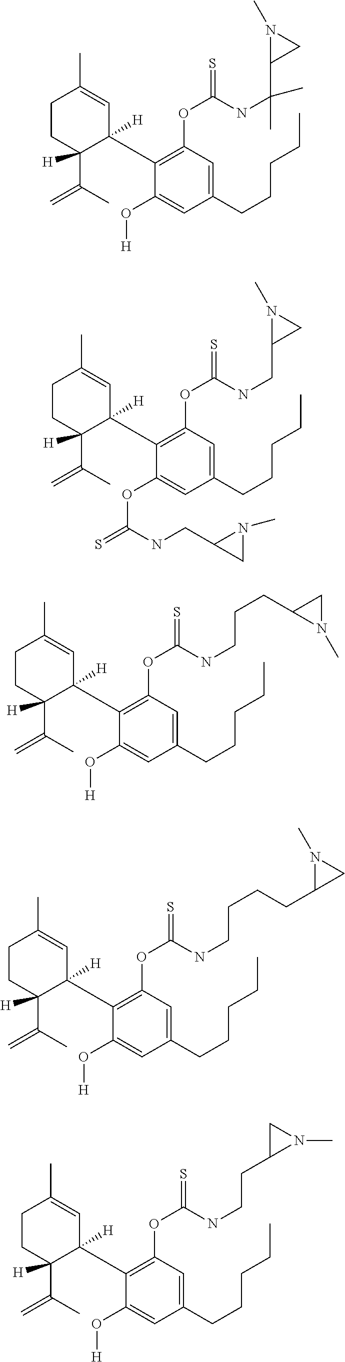

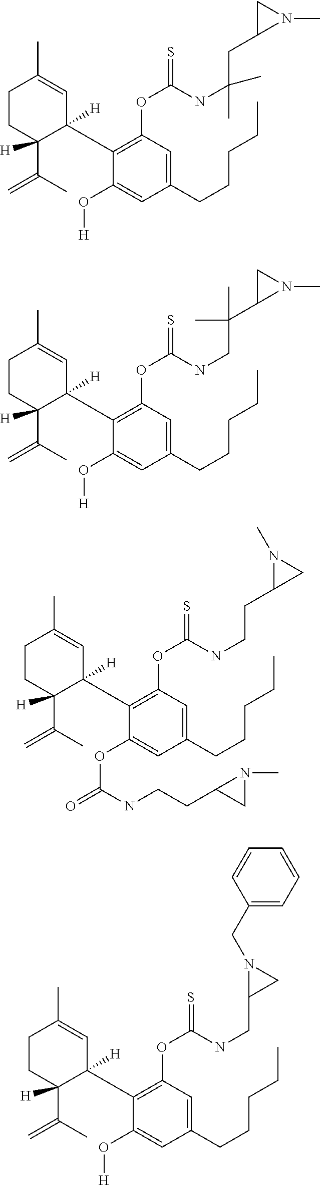

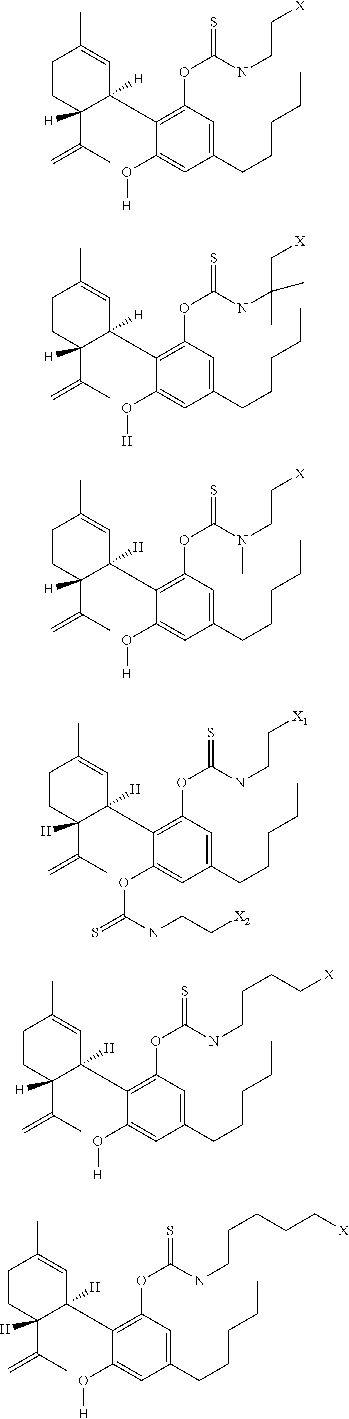

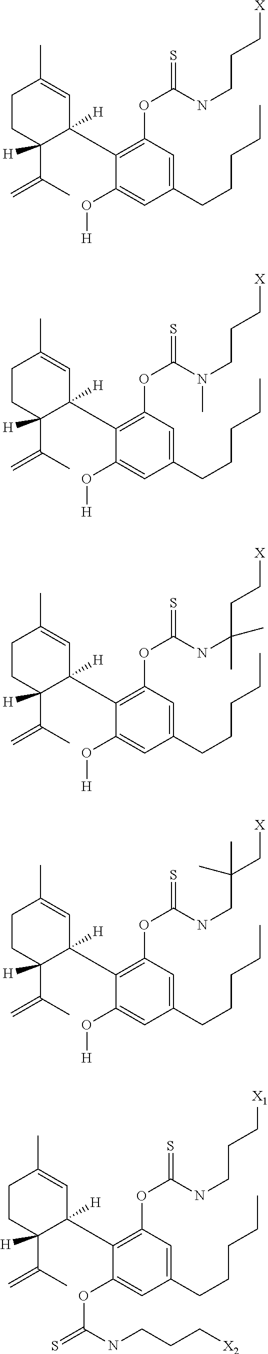

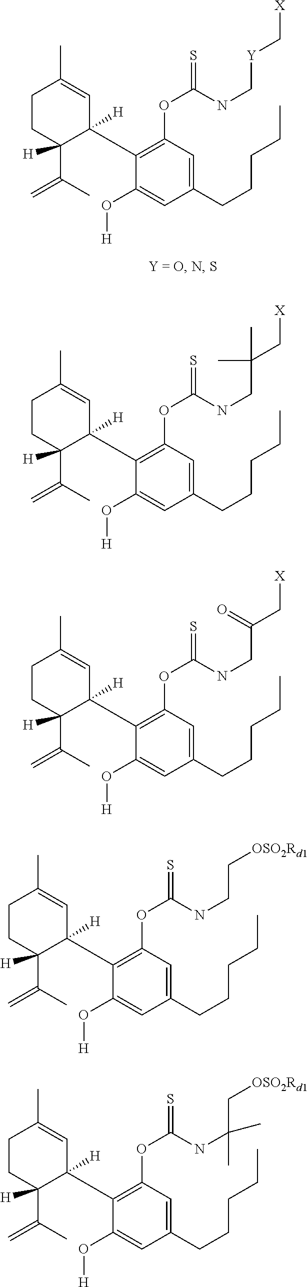

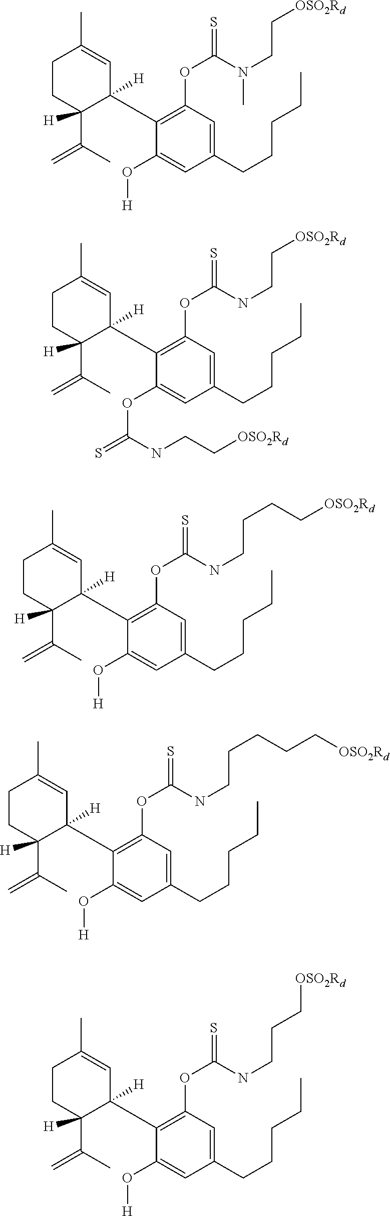

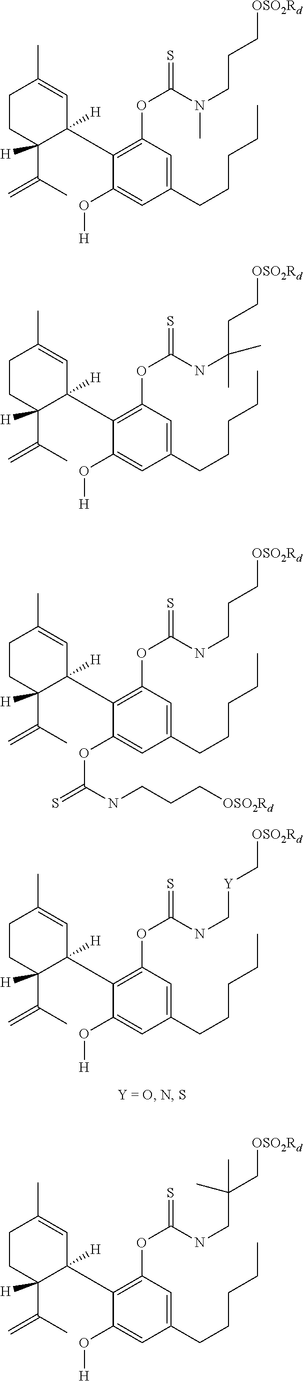

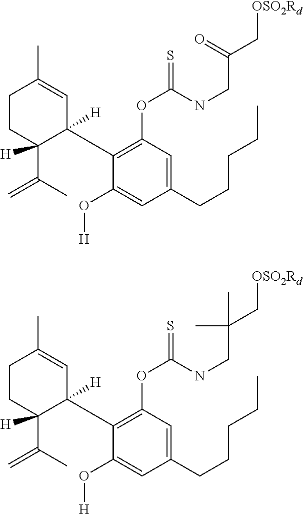

### Thiocarbonate Linkers

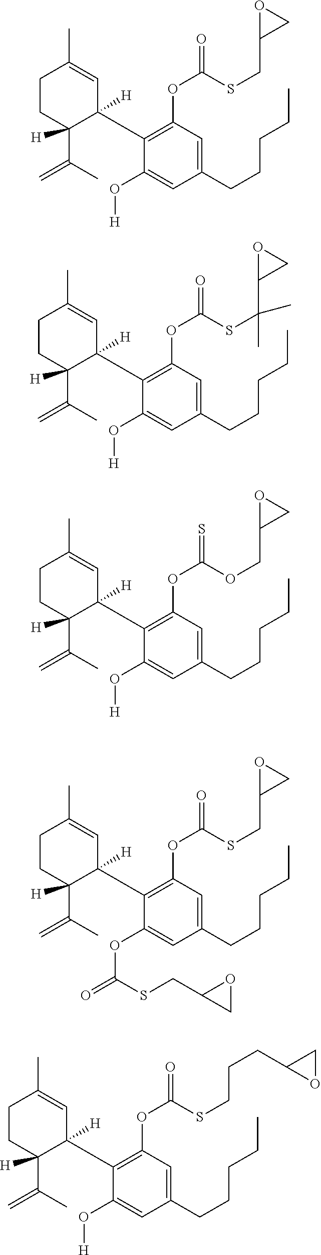 [SMILES: C=C(C)[C@@H]1CCC(C)=C[C@H]1c1c(O)cc(CCCCC)cc1OC(=O)SCC1CO1 ; C=C(C)[C@@H]1CCC(C)=C[C@H]1c1c(O)cc(CCCCC)cc1OC(=O)SC(C)(C)C1CO1 ; C=C(C)[C@@H]1CCC(C)=C[C@H]1c1c(O)cc(CCCCC)cc1OC(=S)OCC1CO1 ; C=C(C)[C@@H]1CCC(C)=C[C@H]1c1c(OC(=O)SCC2CO2)cc(CCCCC)cc1OC(=O)SCC1CO1 ; C=C(C)[C@@H]1CCC(C)=C[C@H]1c1c(O)cc(CCCCC)cc1OC(=O)SCCCC1CO1]

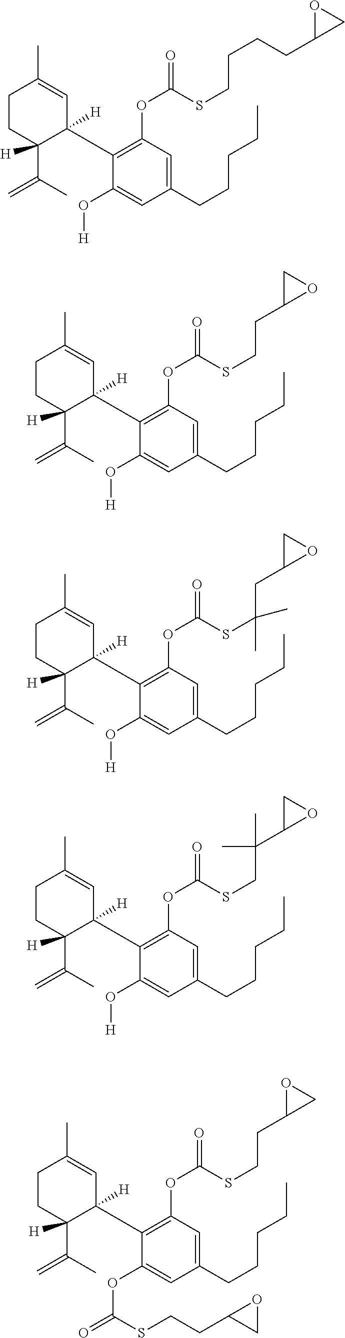 [SMILES: C=C(C)[C@@H]1CCC(C)=C[C@H]1c1c(O)cc(CCCCC)cc1OC(=O)SCCCCC1CO1 ; C=C(C)[C@@H]1CCC(C)=C[C@H]1c1c(O)cc(CCCCC)cc1OC(=O)SCCC1CO1 ; C=C(C)[C@@H]1CCC(C)=C[C@H]1c1c(O)cc(CCCCC)cc1OC(=O)SC(C)(C)CC1CO1 ; C=C(C)[C@@H]1CCC(C)=C[C@H]1c1c(O)cc(CCCCC)cc1OC(=O)SCC(C)(C)C1CO1 ; C=C(C)[C@@H]1CCC(C)=C[C@H]1c1c(OC(=O)SCCC2CO2)cc(CCCCC)cc1OC(=O)SCCC1CO1]

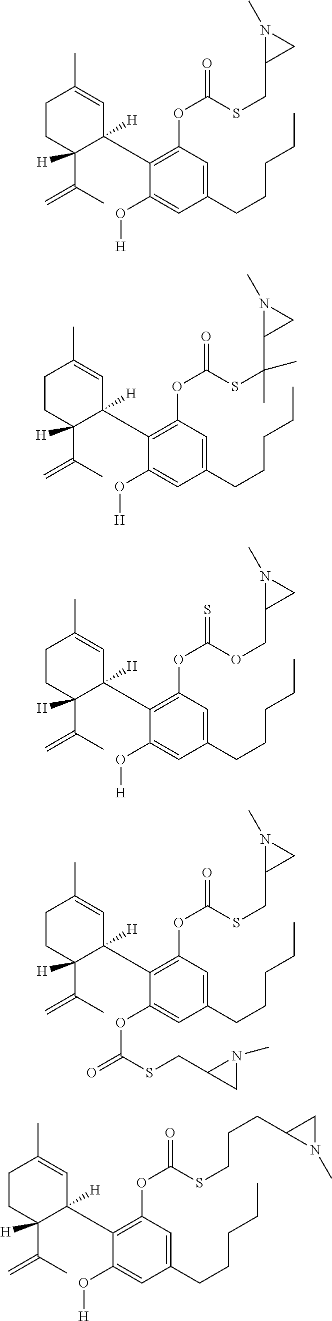

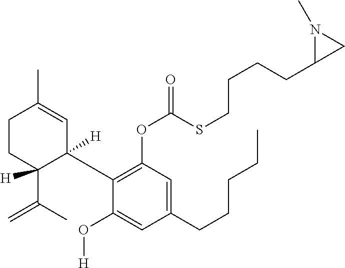 [SMILES: C=C(C)[C@@H]1CCC(C)=C[C@H]1c1c(O)cc(CCCCC)cc1OC(=O)SCCCCC1CN1C]

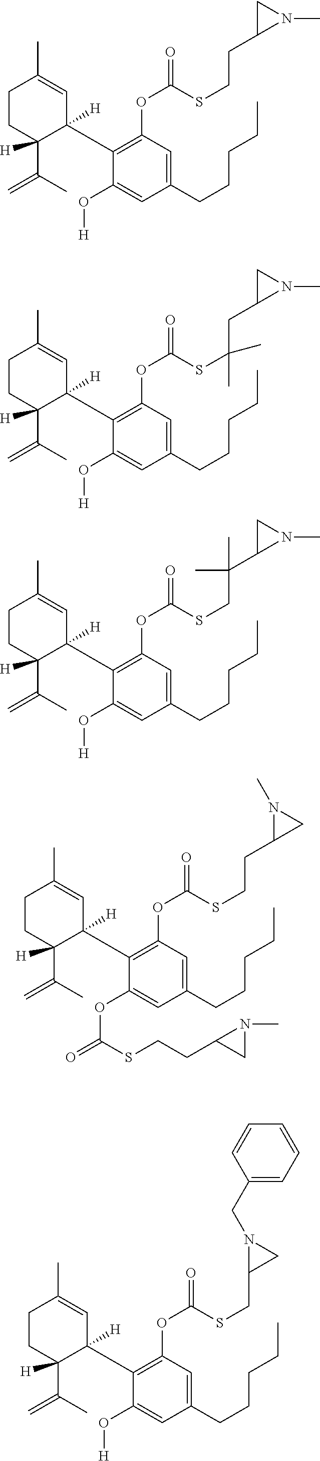 [SMILES: C=C(C)[C@@H]1CCC(C)=C[C@H]1c1c(O)cc(CCCCC)cc1OC(=O)SCCC1CN1C ; C=C(C)[C@@H]1CCC(C)=C[C@H]1c1c(O)cc(CCCCC)cc1OC(=O)SC(C)(C)CC1CN1C ; C=C(C)[C@@H]1CCC(C)=C[C@H]1c1c(O)cc(CCCCC)cc1OC(=O)SCC(C)(C)C1CN1C ; C=C(C)[C@@H]1CCC(C)=C[C@H]1c1c(OC(=O)SCCC2CN2C)cc(CCCCC)cc1OC(=O)SCCC1CN1C ; C=C(C)[C@@H]1CCC(C)=C[C@H]1c1c(O)cc(CCCCC)cc1OC(=O)SCC1CN1Cc1ccccc1]

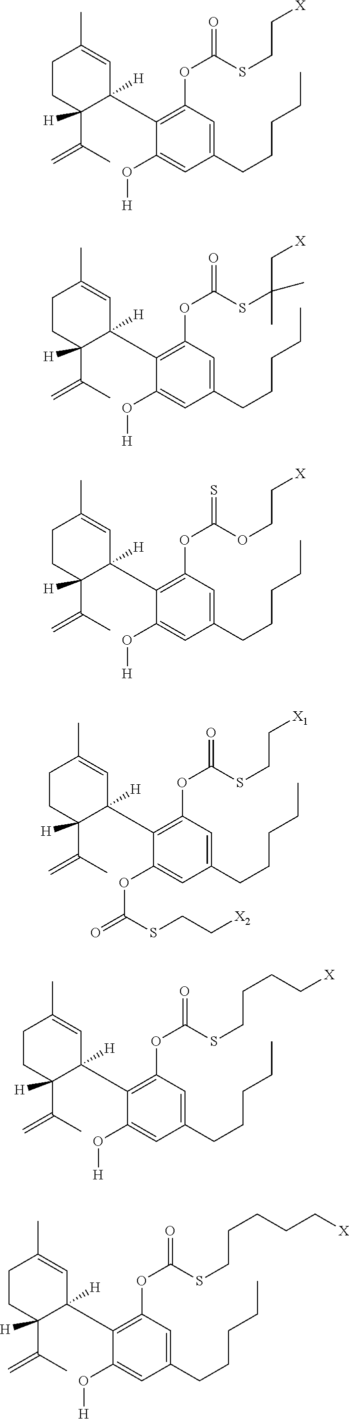

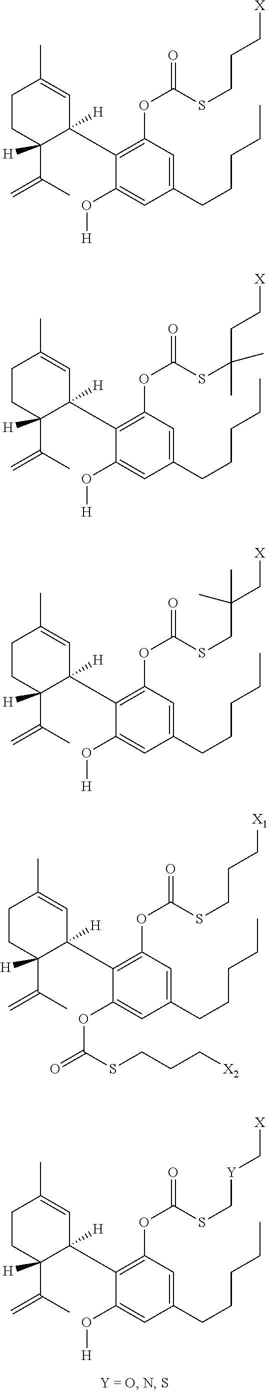

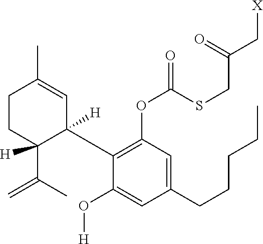

Pharmaceutical Compositions, Routes of Administration, and Dosages

One or more conjugate molecules, which can be the same or different, can be provided in a pharmaceutical composition together with a pharmaceutically acceptable vehicle. The “pharmaceutically acceptable vehicle” can comprise one or more substances which do not affect the biological activities of the conjugate molecules and, when administered to a patient, do not cause an adverse reaction. Excipients, such as calcium carbonate, calcium phosphate, various sugars and types of starch, cellulose derivatives, and gelatin can be included. Pharmaceutically acceptable vehicles for liquid compositions include, but are not limited to, water, saline, polyalkylene glycols (e.g., polyethylene glycol), vegetable oils, and hydrogenated naphthalenes. Controlled release, for example, can be achieved using biocompatible, biodegradable polymers of lactide or copolymers of lactide/glycolide or polyoxyethylene/polyoxypropylene.

Methods of preparing pharmaceutical compositions are well known. Pharmaceutical compositions can be prepared as solids, semi-solids, or liquid forms, such as tablets, capsules, powders, granules, ointments, solutions, suspensions, emulsions, suppositories, injections, inhalants, gels, microspheres, aerosols, and mists. Liquid pharmaceutical compositions can be lyophilized. Lyophilized compositions can be provided in a kit with a suitable liquid, typically water for injection (WFI) for use in reconstituting the composition.

Typical administration routes include, but are not limited to, oral, topical, transdermal, inhalation, parenteral, sublingual, buccal, rectal, vaginal, and intranasal.

The dose of a pharmaceutical composition can be based on the doses typically used for the particular therapeutic agent(s) which provide the therapeutic agent component(s) of a conjugate molecule. These doses are well known in the art.

Therapeutic Methods

The disclosed conjugate molecules have a variety of therapeutic uses depending on which therapeutic agent component(s) are included in a conjugate molecule. “Treat” as used in this disclosure means reducing or inhibiting the progression of one or more symptoms of the disorder or disease for which the conjugate molecule is administered, such as inflammation or pain.

In come embodiments, conjugate molecules are particularly useful for treating proliferative disorders, including cancer. For example, treatment of cancer may include inhibiting the progression of a cancer, for example, by reducing proliferation of neoplastic or pre-neoplastic cells; destroying neoplastic or pre-neoplastic cells; or inhibiting metastasis or decreasing the size of a tumor. Cancers that can be treated include, but are not limited to, multiple myeloma (including systemic light chain amyloidosis and Waldenström's macroglobulinemia/lymphoplasmocytic lymphoma), myelodysplastic syndromes, myeloproliferative neoplasms, gastrointestinal malignancies (e.g., esophageal, esophagogastric junction, gallbladder, gastric, colon, pancreatic, hepatobiliary, anal, and rectal cancers), leukemias (e.g., acute myeloid, acute myelogenous, chronic myeloid, chronic myelogenous, acute lymphocytic, acute lymphoblastic, chronic lymphocytic, and hairy cell leukemia), Hodgkin lymphoma, non-Hodgkin's lymphomas (e.g., B-cell lymphoma, hairy cell leukemia, primary cutaneous B-cell lymphoma, and T-cell lymphoma), lung cancer (e.g., small cell and non-small cell lung cancers), basal cell carcinoma, plasmacytoma, breast cancer, bladder cancer, kidney cancer, neuroendocrine tumors, adrenal tumors, bone cancer, soft tissue sarcoma, head and neck cancer, thymoma, thymic carcinoma, cervical cancer, uterine cancers, ovarian cancer (e.g., Fallopian tube and primary peritoneal cancers), vaginal cancer, vulvar cancer, penile cancer, testicular cancer, prostate cancer, melanoma (e.g., cutaneous and uveal melanomas), non-melanoma skin cancers (e.g., basal cell skin cancer, dermatofibrosarcoma protuberans, Merkel cell carcinoma, and squamous cell skin cancer), malignant pleural mesothelioma, central nervous system (CNS) cancers (e.g., astrocytoma, oligodendroglioma, anaplastic glioma, glioblastoma, intra-cranial ependymoma, spinal ependymoma, medulloblastoma, CNS lymphoma, spinal cord tumor, meningioma, brain metastases, leptomeningeal metastases, metastatic spine tumors), and occult primary cancers (i.e., cancers of unknown origin).

Conjugate molecules described herein can be administered in conjunction with one or more other cancer therapies such as chemotherapies, immunotherapies, tumor-treating fields (TTF; e.g., OPTUNE® system), radiation therapies (XRT), and other therapies (e.g., hormones, autologous bone marrow transplants, stem cell reinfusions). “In conjunction with” includes administration together with, before, or after administration of the one or more other cancer therapies.

Chemotherapies include, but are not limited to, FOLFOX (leucovorin calcium, fluorouracil, oxaliplatin), FOLFIRI (leucovorin calcium, fluorouracil, irinotecan), FOLFIRINOX (leucovorin calcium, fluorouracil, irinotecan, oxaliplatin), irinotecan (e.g., CAMPTOSAR®), capecitabine (e.g., XELODA®), gemcitabine (e.g., GEMZAR®), paclitaxel (e.g., ABRAXANE®), dexamethasone, lenalidomide (e.g., REVLIMID®), pomalidomide (e.g., POMALYST®), cyclophosphamide, regorafenib (e.g., STIVARGA®), erlotinib (e.g., TARCEVA®), ixazomib (e.g., NINLARO®), bevacizumab (e.g., AVASTIN®), bortezomib (e.g., VELCADE®, NEOMIB®), cetuximab (e.g., ERBITUX®), daratumumab (e.g., DARZALEX®), elotumumab (e.g., EMPLICITI™), carfilzomib (e.g., KYPROLIS®), palbociclib (e.g., IBRANCE®), fulvestrant (e.g., FASLODEX®), carboplatin, cisplatin, taxol, nab paclitaxel (e.g., ABRAXANE®), 5-fluorouracil, RVD (lenalidomide, bortezomib, dexamethasone), pomolidamide (e.g., POMALYST®), temozolomide (e.g., TEMODAR®), PCV (procarbazine, lomustine, vincristine), methotrexate (e.g., TREXALL®, RASUV®, XATMEP®), carmustine (e.g., BICNU®, GLIADEL WAFER®), etoposide (e.g., ETOPOPHOS®, TOPOSAR®), sunitinib (e.g., SUTENT®), everolimus (e.g., ZORTRESS®, AFINITOR®), rituximab (e.g., RITUXAN®, MABTHERA®), R-MPV (vincristine, procarbazine, rituximab), cytarabine (e.g., DEPOCYT®, CYTOSAR-U®), thiotepa (e.g., TEPADINA®), busulfan (e.g., BUSULFEX®, MYLERAN®), TBC (thiotepa, busulfan, cyclophosphamide), ibrutinib (e.g., IMBRUVICA®), topotecan (e.g., HYCAMTIN®), pemetrexed (e.g., ALIMTA®), vemurafenib (e.g., ZELBORAF®), cobimetinib (e.g., COTELLIC®), dabrafenib (e.g., TAFINLAR®), trametinib (e.g., MEKINIST®), alectinib (e.g., ALECENSA®), lapatinib (e.g., TYKERB®), neratinib (e.g., NERLYNX®), ceritinib (e.g., ZYKADIA®), brigatinib (e.g., ALUNBRIG®), afatinib (e.g., GILOTRIF®, GIOTRIF®), gefitinib (e.g., IRESSA®), osimertinib (e.g., TAGRISSO®, TAGRIX®), and crizotinib (e.g., XALKORI®).

Immunotherapies include, but are not limited to, checkpoint inhibitors, including monoclonal antibodies such as ipilimumab (e.g., YERVOY®), nivolumab (e.g., OPDIVO®), pembrolizumab (e.g., KEYTRUDA®); cytokines; cancer vaccines; and adoptive cell transfer.

In some embodiments, one or more conjugate molecules described above are administered to a patient with a cancer, including any of those cancers listed above. In some embodiments, as described below, the patient has colon cancer, rectal cancer, pancreatic cancer, multiple myeloma, or glioblastoma multiforme and the conjugate molecule(s) are administered in conjunction with an additional therapy appropriate for the particular cancer.

The disclosed conjugate molecules can be used to treat these and other disorders in the same way the therapeutic agent components of the molecules are used, and these methods are well known. For example, conjugate molecules containing entecavir, emtricitabine, daclatasvir, atazanavir, didanosine, and/or stavudine can be used to treat viral infections; conjugate molecules containing diclofenac or celecoxib components can be used as anti-inflammatory agents; conjugate molecules containing a warfarin component can be used as anticoagulants; and conjugate molecules containing pravastatin components can be used to treat cardiovascular disorders. An advantage of conjugate molecules, however, is that the cannabinoid can be delivered directly to the site of action of the therapeutic agent, where the released cannabinoid can provide further therapeutic benefits. The therapeutic benefits and potential benefits of cannabinoids are well known. For example, see Dzierzanowski, Cancers 11, 129-41, 2019 (oncology and palliative care); Urits et al., Pain Ther. 8, 41-51, 2019 (pain); Hillen et al., Ther. Adv. Drug Safety 10, 1-23 2019 (neuropsychiatric symptoms in dementia).

### EXAMPLES

The following procedures for synthesizing various types and classes of compounds are general representative procedures for building in the primary functionality of the compounds. The reagent system, reaction conditions, and protecting group strategy may vary for any specific analog. Specific building blocks vary in accordance with the specific desired product. The bromide compounds may be synthesized as corresponding chloride or iodide compounds. The procedures below show cannabidiol (CBD) as a representative cannabinoid, although other cannabinoids containing hydroxyl groups may be substituted to generate alternative analogs.

### Example 1. Epoxide-Containing Conjugate Molecules

Epoxide carbamate linked compounds are synthesized as follows. A cannabinoid (CBD in this example) is reacted with phosgene (or a suitable phosgene surrogate) and an aminoepoxide ([5689-75-8] in this example) under standard basic conditions to form the desired carbamate linked product.

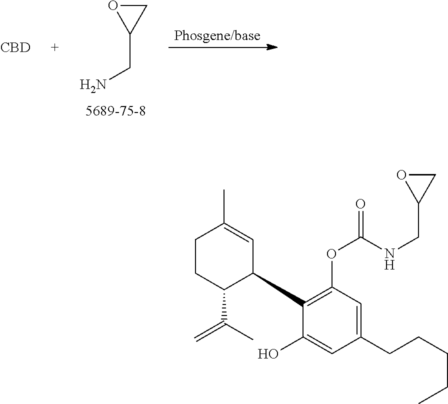 [SMILES: "CBD" ; "+" ; NCC1CO1 ; C=C(C)[C@@H]1CCC(C)=C[C@H]1c1c(O)cc(CCCCC)cc1OC(=O)NCC1CO1]

Epoxide carbonate linked compounds are synthesized as follows. A cannabinoid (CBD in this example) is reacted with phosgene (or a suitable phosgene surrogate) and a hydroxyepoxide ([556-52-5] in this example) under standard basic conditions to form the desired carbonate linked product.

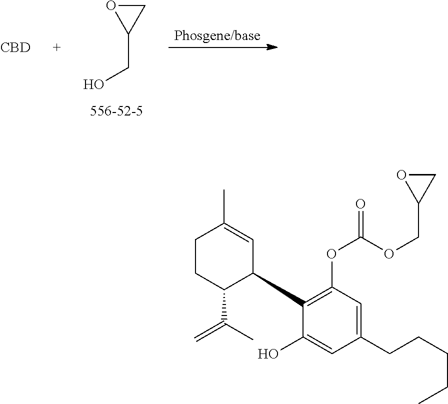 [SMILES: "CBD" ; "+" ; OCC1CO1 ; C=C(C)[C@@H]1CCC(C)=C[C@H]1c1c(O)cc(CCCCC)cc1OC(=O)OCC1CO1]

Epoxide ester linked compounds are synthesized as follows. A cannabinoid (CBD in this example) is esterified under standard conditions, in this example with the epoxy acid building block [86310-98-7] to give the desired product.

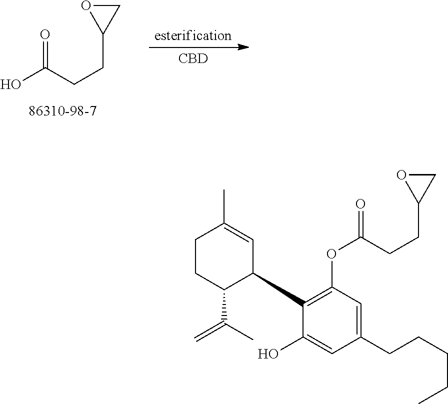 [SMILES: O=C(O)CCC1CO1 ; "CBD" ; C=C(C)[C@@H]1CCC(C)=C[C@H]1c1c(O)cc(CCCCC)cc1OC(=O)CCC1CO1]

Epoxide imidate linked compounds are synthesized as follows. A cannabinoid (CBD in this example) is reacted with an imidocarbonyl chloride (in this case [5652-90-4]) and a hydroxyepoxide ([556-52-5] in this example) under standard basic conditions to form the desired imidate linked product.

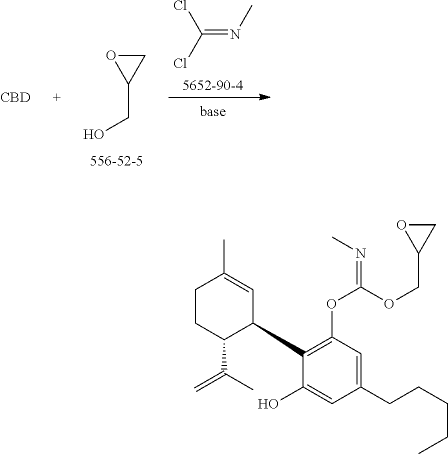

Epoxide isourea linked compounds are synthesized as follows. A cannabinoid (CBD in this example) is reacted with an imidocarbonyl chloride (in this case [5652-90-4]) and an aminoepoxide ([5689-75-8] in this example) under standard basic conditions to form the desired isourea linked product.

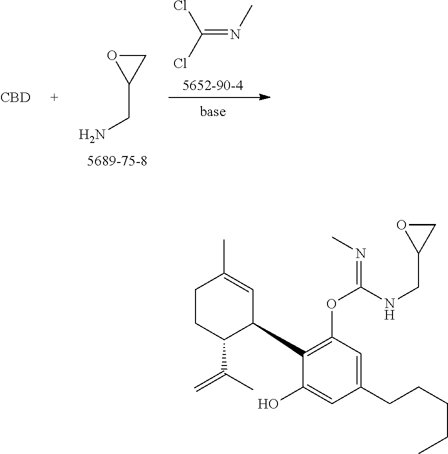

Epoxide phosphorodiamide linked compounds are synthesized as follows. Using conditions similar to those referenced in the Scheme, N,N-Dimethylphosphoramidodichloridate ([677-43-0]) is reacted with an aminoepoxide ([5689-75-8] in this example). The adduct is then reacted with a cannabinoid (CBD in this example) under standard basic conditions to form the desired product.

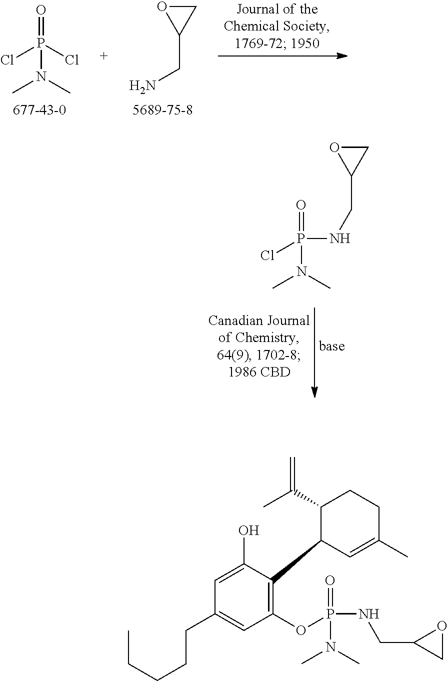

Epoxide S-alkyl thiocarbonate linked compounds are synthesized as follows. A cannabinoid (CBD in this example) is reacted with phosgene (or a suitable phosgene surrogate) and a thiol-epoxide ([45357-98-0] in this example) under standard basic conditions to form the desired S-alkyl thiocarbonate linked product.

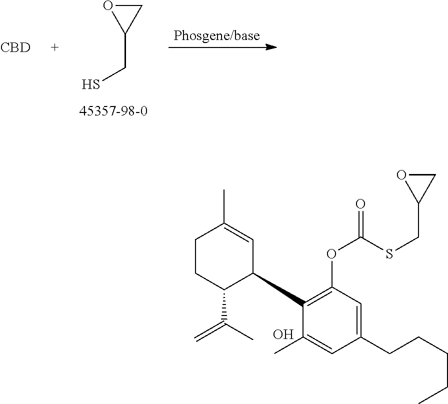

Epoxide thiocarbamate linked compounds are synthesized as follows. A cannabinoid (CBD in this example) is reacted with thiophosgene (or a suitable thiophosgene surrogate) and an aminoepoxide ([5689-75-8] in this example) under standard basic conditions to form the desired thiocarbamate linked product.

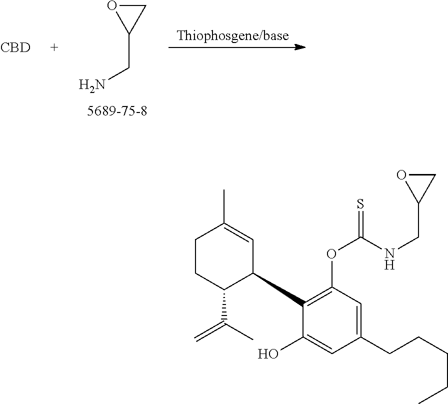 [SMILES: "CBD" ; "+" ; NCC1CO1 ; C=C(C)[C@@H]1CCC(C)=C[C@H]1c1c(O)cc(CCCCC)cc1OC(=S)NCC1CO1]

Epoxide thiocarbonate linked compounds are synthesized as follows. A cannabinoid (CBD in this example) is reacted with thiophosgene (or a suitable thiophosgene surrogate) and a hydroxyepoxide ([556-52-5] in this example) under standard basic conditions to form the desired thiocarbonate linked product.

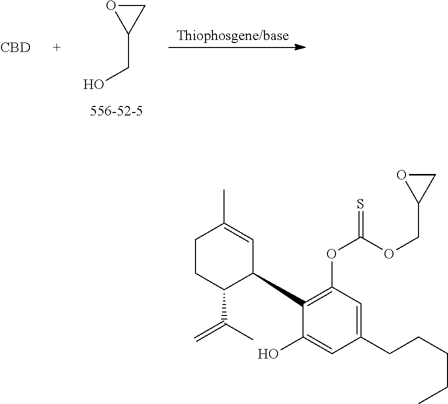 [SMILES: "CBD" ; "+" ; OCC1CO1 ; C=C(C)[C@@H]1CCC(C)=C[C@H]1c1c(O)cc(CCCCC)cc1OC(=S)OCC1CO1]

Epoxide thioimidate linked compounds are synthesized as follows. A cannabinoid (CBD in this example) is reacted with an imidocarbonyl chloride (in this case [5652-90-4]) and a thiol-epoxide ([45357-98-0] in this example) under standard basic conditions to form the desired thioimidate linked product.

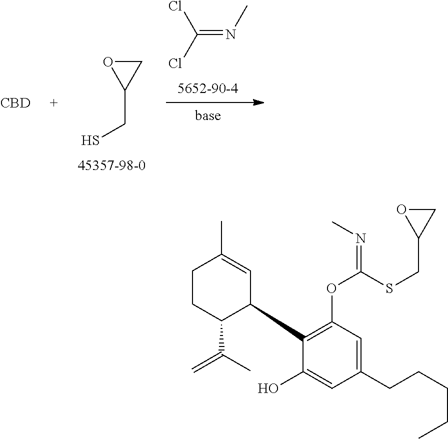

Epoxide thiophosphinodiamide linked compounds are synthesized as follows. Using conditions similar to those referenced in the Scheme, dimethylphosphoramidothioic dichloride ([1498-65-3]) is reacted with an aminoepoxide ([5689-75-8] in this example). The adduct is then reacted with a cannabinoid (CBD in this example) under standard basic conditions, to form the desired product.

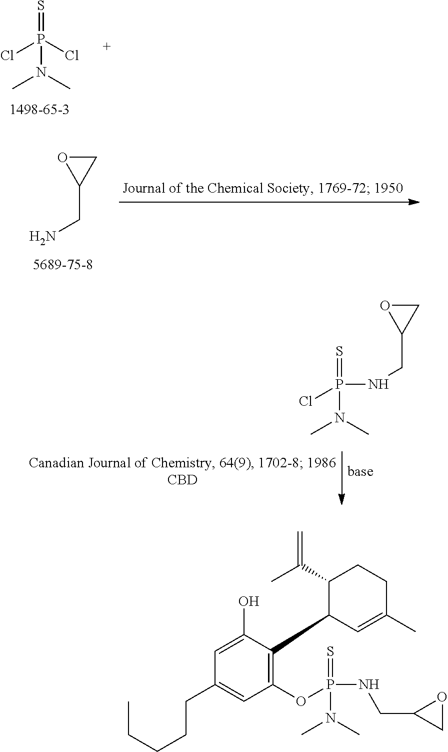

Epoxide xanthate linked compounds are synthesized as follows. A cannabinoid (CBD in this example) is reacted with thiophosgene (or a suitable thiophosgene surrogate) and a thiol-epoxide ([45357-98-0] in this example) under standard basic conditions to form the desired xanthate linked product.

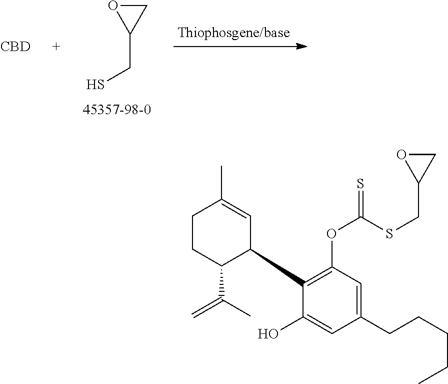 [SMILES: "CBD" ; SCC1CO1 ; C=C(C)[C@@H]1CCC(C)=C[C@H]1c1c(O)cc(CCCCC)cc1OC(=S)SCC1CO1]

### Example 2. Aziridine-Containing Conjugate Molecules

Aziridine carbamate linked compounds are synthesized as follows. A cannabinoid (CBD in this example) is reacted with phosgene (or a suitable phosgene surrogate) and an aminoaziridine ([88714-40-3] in this example) under standard basic conditions to form the desired carbamate linked product.

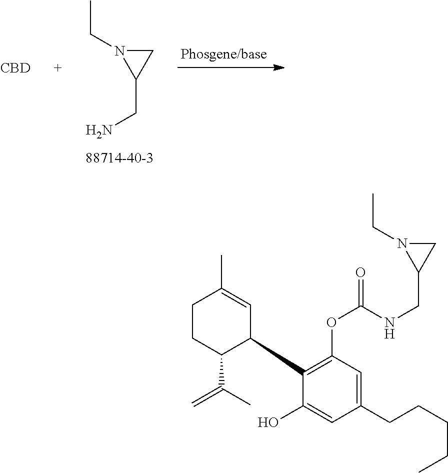

Aziridine carbonate linked compounds are synthesized as follows. A cannabinoid (CBD in this example) is reacted with phosgene (or a suitable phosgene surrogate) and a hydroxyaziridine ([25662-15-1] in this example) under standard basic conditions to form the desired carbonate linked product.

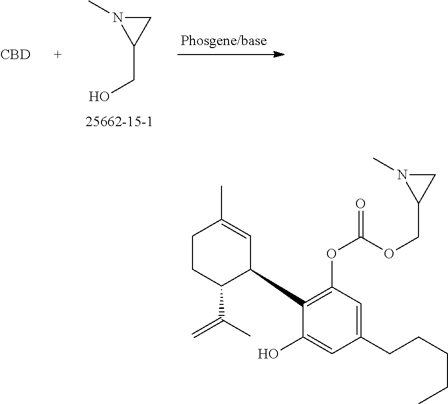

Aziridine ester linked compounds are synthesized as follows. The previously reported hydroxymethyl building block [126587-35-7] is treated with base, in this example sodium hydride, to generate the aziridinyl intermediate. Removal of the BOC protecting group followed by alkylation of the resulting amine gives the alkyl aziridine-ester intermediate. Standard hydrolysis of the ester gives the carboxylic acid precursor, which is esterified with the cannabinoid under standard esterification conditions to give the desired product.

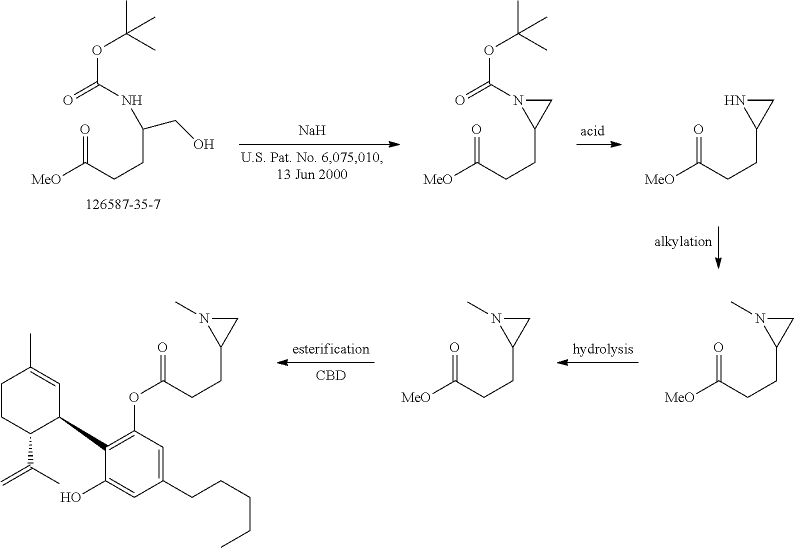

Aziridine imidate linked compounds are synthesized as follows. A cannabinoid (CBD in this example) is reacted with an imidocarbonyl chloride (in this case [5652-90-4]) and a hydroxyaziridine ([25662-15-1] in this example) under standard basic conditions to form the desired imidate linked product.

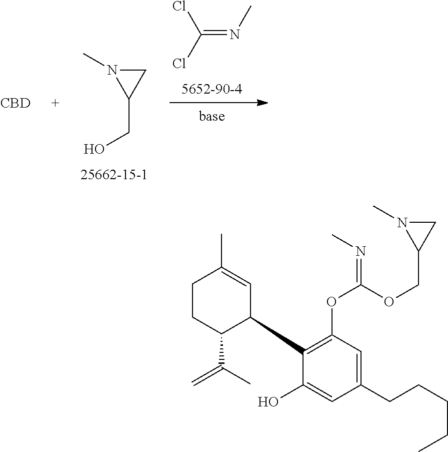

Aziridine isourea linked compounds are synthesized as follows. A cannabinoid (CBD in this example) is reacted with an imidocarbonyl chloride (in this case [5652-90-4]) and an aminoaziridine ([88714-40-3] in this example) under standard basic conditions to form the desired isourea linked product.

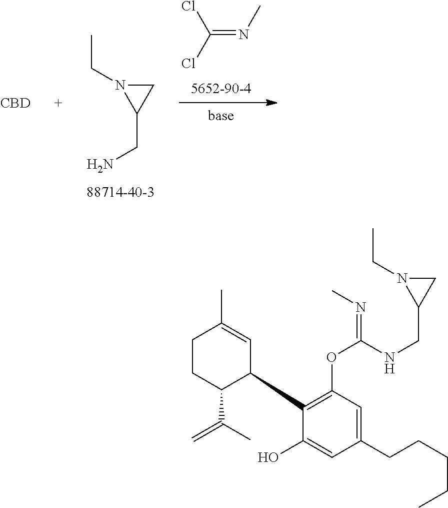

Aziridine phosphorodiamide linked compounds are synthesized as follows. Using conditions similar to those referenced in the Scheme, N,N-Dimethylphosphoramidodichloridate ([677-43-0]) is reacted with an aminoaziridine ([88714-40-3] in this example). The adduct is then reacted with a cannabinoid (CBD in this example) under standard basic conditions to form the desired product.

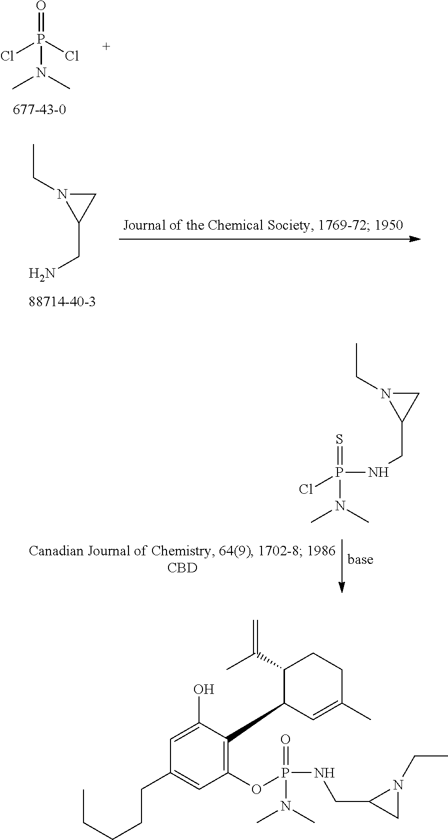

Aziridine thiocarbamate linked compounds are synthesized as follows. A cannabinoid (CBD in this example) is reacted with thiophosgene (or a suitable thiophosgene surrogate) and an aminoaziridine ([88714-40-3] in this example) under standard basic conditions to form the desired thiocarbamate linked product.

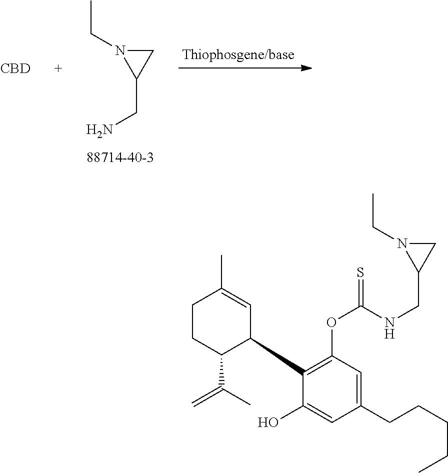

Aziridine thiocarbonate linked compounds are synthesized as follows. A cannabinoid (CBD in this example) is reacted with thiophosgene (or a suitable thiophosgene surrogate) and a hydroxyaziridine ([25662-15-1] in this example) under standard basic conditions to form the desired thiocarbonate linked product.

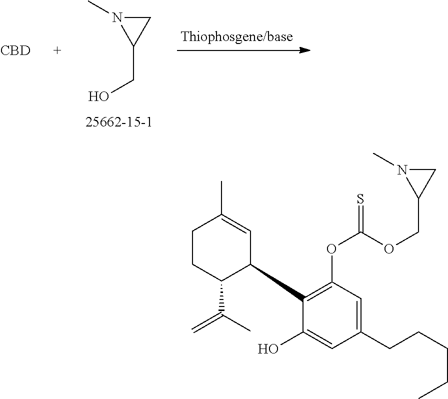

Aziridine thiophosphinodiamide linked compounds are synthesized as follows. Using conditions similar to those referenced in the Scheme, dimethylphosphoramidothioic dichloride ([1498-65-3]) is reacted with an aminoaziridine ([88714-40-3] in this example). The adduct is then reacted with a cannabinoid (CBD in this example) under standard basic conditions, to form the desired product.

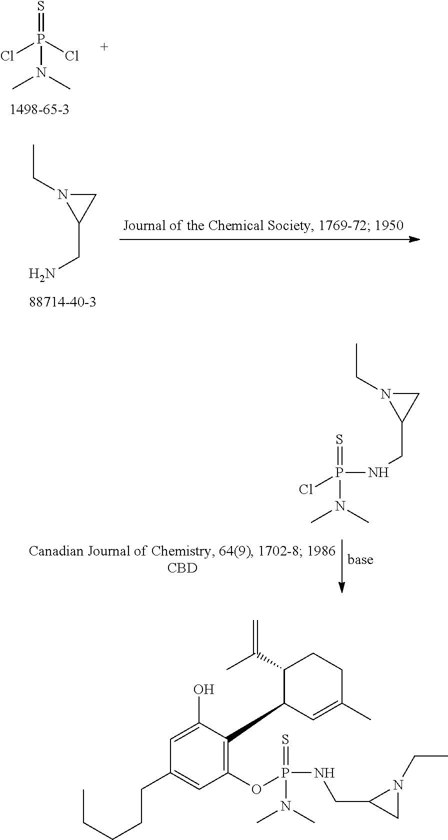

Sulfonate carbamate linked compounds are synthesized as follows. A cannabinoid (CBD in this example) is reacted with phosgene (or a suitable phosgene surrogate) and an amino-alcohol ([156-87-6] in this example) under standard basic conditions to form the carbamate linked intermediate. Reaction with a sulfonyl chloride, in this case mesyl chloride, gives the desired product.

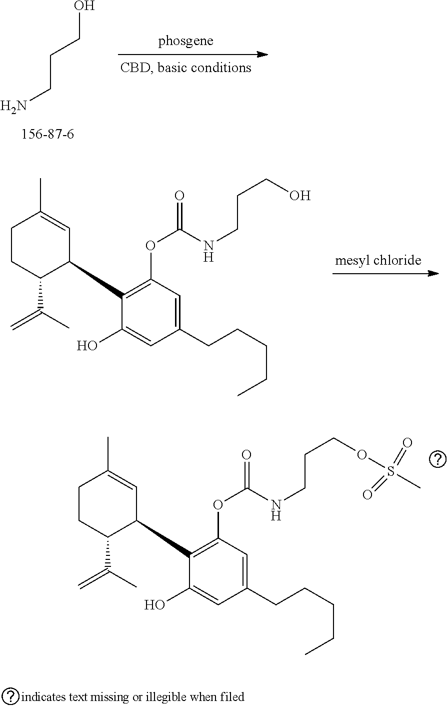 [SMILES: NCCCO ; C=C(C)[C@@H]1CCC(C)=C[C@H]1c1c(O)cc(CCCCC)cc1OC(=O)NCCCO ; C=C(C)[C@@H]1CCC(C)=C[C@H]1c1c(O)cc(CCCCC)cc1OC(=O)NCCCOS(C)(=O)=O]

Sulfonate carbonate linked compounds are synthesized as follows. A diol compound, in this case 1,3-propanediol [13392-69-3] is reacted with a sulfonyl chloride, in this case tosyl chloride, to give the monosulfonate intermediate. Reaction of the remaining hydroxyl group in this intermediate with phosgene (or a suitable surrogate) and a cannabinoid (CBD in this example) under standard basic conditions forms the desired carbonate linked product.

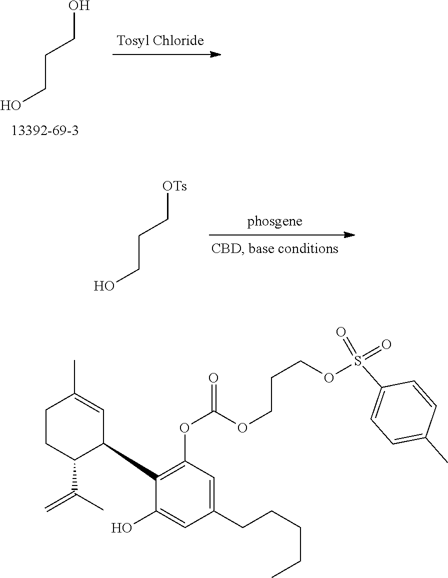 [SMILES: OCCCO ; Cc1ccc(S(=O)(=O)OCCCO)cc1 ; C=C(C)[C@@H]1CCC(C)=C[C@H]1c1c(O)cc(CCCCC)cc1OC(=O)OCCCOS(=O)(=O)c1ccc(C)cc1]

Sulfonate ester linked compounds are synthesized as follows. A hydroxyacid starting material, in this case [13392-69-3], is esterified under referenced conditions for selective esterification of an aromatic OH in the presence of an aliphatic OH. The ester linked intermediate then undergoes sulfonylation, in this case with mesyl chloride, under referenced conditions to give the desired product.

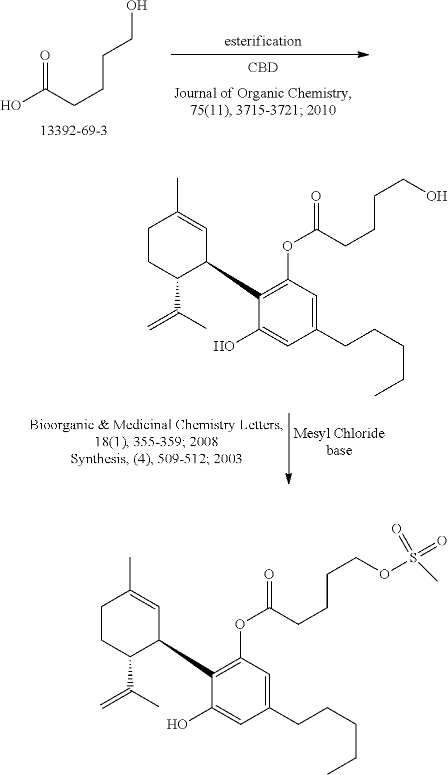 [SMILES: O=C(O)CCCCO ; "CBD" ; C=C(C)[C@@H]1CCC(C)=C[C@H]1c1c(O)cc(CCCCC)cc1OC(=O)CCCCO ; C=C(C)[C@@H]1CCC(C)=C[C@H]1c1c(O)cc(CCCCC)cc1OC(=O)CCCCOS(C)(=O)=O]

Sulfonate imidate linked compounds are synthesized as follows. A diol compound, in this case 1,3-propanediol [13392-69-3] is reacted with a sulfonyl chloride, in this case tosyl chloride, to give the monosulfonate intermediate. Reaction of the remaining hydroxyl group in this intermediate with an imidocarbonyl chloride (in this case [5652-90-4]) under standard basic conditions forms the desired imidate linked product.

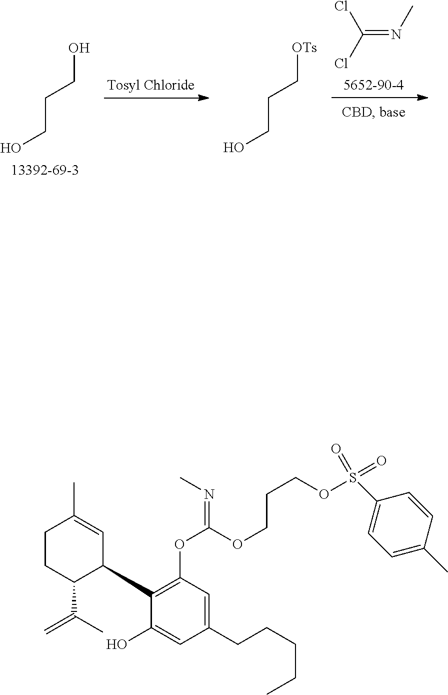

Sulfonate isourea linked compounds are synthesized as follows. A cannabinoid (CBD in this example) is reacted with an imidocarbonyl chloride (in this case [5652-90-4]) and an amino-alcohol ([156-87-6] in this example) under standard basic conditions to form the isourea linked intermediate. Sulfonylation, in this case with mesyl chloride, under referenced conditions (see sulfonate ester above) gives the desired product.

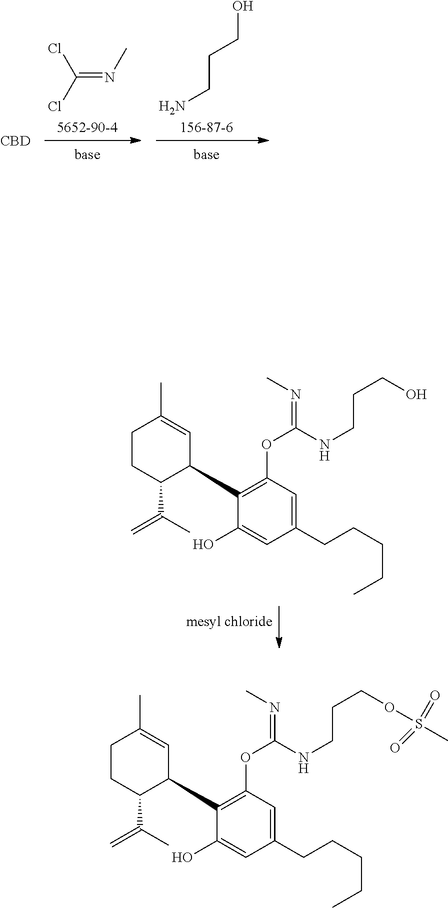

Sulfonate phosphorodiamide linked compounds are synthesized as follows. Using conditions similar to those referenced in the epoxide phosphorodiamide Scheme, N,N-Dimethylphosphoramidodichloridate ([677-43-0]) is reacted with a cannabinoid (CBD in this example) and an amino-alcohol ([156-87-6] in this example). The adduct then undergoes sulfonylation, in this case with mesyl chloride, under referenced conditions (see sulfonate ester above) gives the desired product.

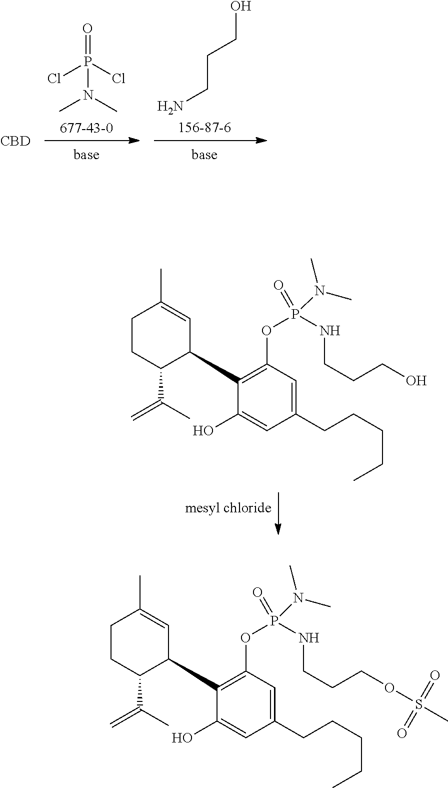

Sulfonate S-alkyl thiocarbonate linked compounds are synthesized as follows. A cannabinoid (CBD in this example) is reacted with phosgene (or a suitable phosgene surrogate) and a thiol-alcohol ([19721-22-3] in this example) under standard basic conditions, to form the S-alkyl thiocarbonate linked intermediate. Sulfonylation, in this case with tosyl chloride, gives the desired product.

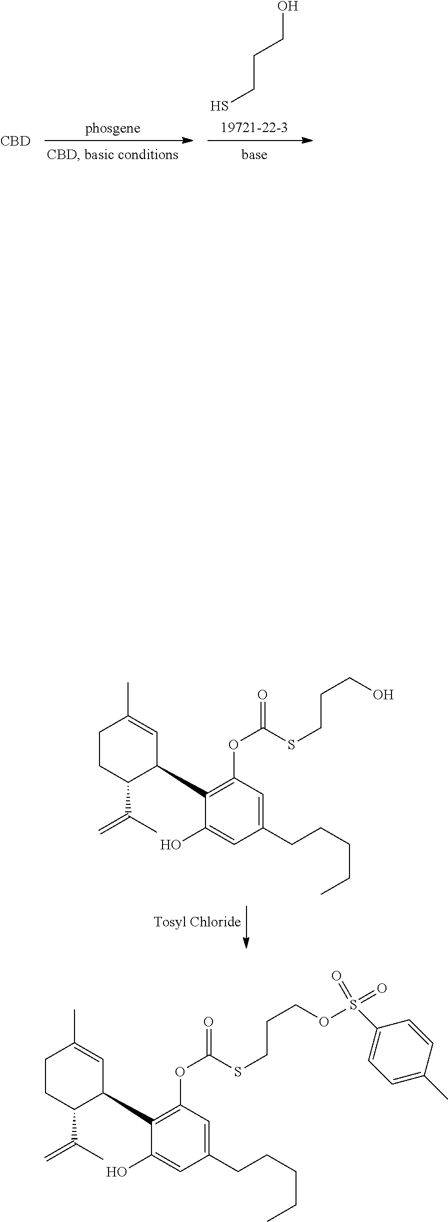 [SMILES: OCCCS ; "CBD" ; C=C(C)[C@@H]1CCC(C)=C[C@H]1c1c(O)cc(CCCCC)cc1OC(=O)SCCCO ; C=C(C)[C@@H]1CCC(C)=C[C@H]1c1c(O)cc(CCCCC)cc1OC(=O)SCCCOS(=O)(=O)c1ccc(C)cc1]

Sulfonate thiocarbamate linked compounds are synthesized as follows. A cannabinoid (CBD in this example) is reacted with thiophosgene (or a suitable thiophosgene surrogate) and an amino-alcohol ([156-87-6] in this example) under standard basic conditions to form the thiocarbamate linked intermediate. Sulfonylation, in this case with mesyl chloride, under referenced conditions (see sulfonate ester above) gives the desired product.

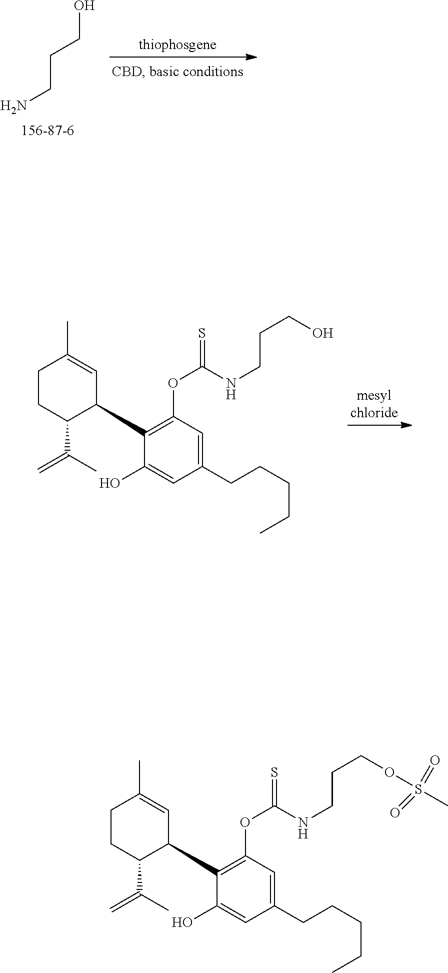 [SMILES: NCCCO ; C=C(C)[C@@H]1CCC(C)=C[C@H]1c1c(O)cc(CCCCC)cc1OC(=S)NCCCO ; C=C(C)[C@@H]1CCC(C)=C[C@H]1c1c(O)cc(CCCCC)cc1OC(=S)NCCCOS(C)(=O)=O]

Sulfonate thiocarbonate linked compounds are synthesized as follows. A diol compound, in this case 1,3-propanediol [13392-69-3] is reacted with a sulfonyl chloride, in this case tosyl chloride, to give the monosulfonate intermediate. Reaction of the remaining hydroxyl group in this intermediate with thiophosgene (or a suitable thiophosgene surrogate) and a cannabinoid (CBD in this example) under standard basic conditions forms the desired thiocarbonate linked product.

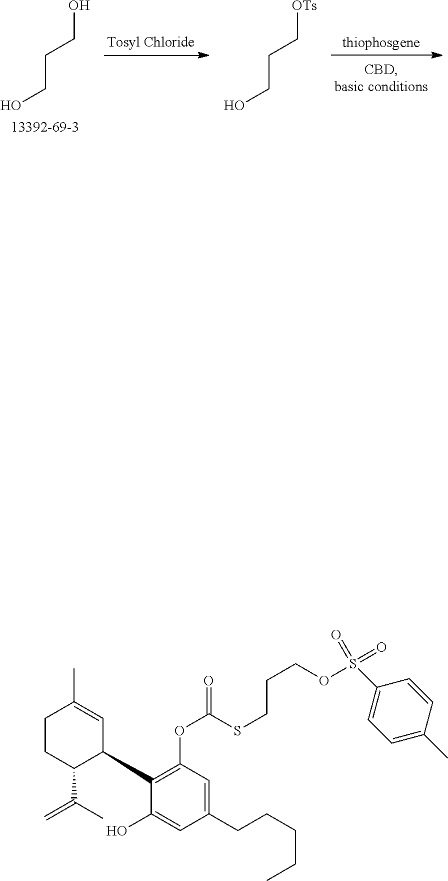 [SMILES: OCCCO ; Cc1ccc(S(=O)(=O)OCCCO)cc1 ; C=C(C)[C@@H]1CCC(C)=C[C@H]1c1c(O)cc(CCCCC)cc1OC(=O)SCCCOS(=O)(=O)c1ccc(C)cc1]

Sulfonate thioimidate linked compounds are synthesized as follows. A cannabinoid (CBD in this example) is reacted with an imidocarbonyl chloride (in this case [5652-90-4]) and a thiol-alcohol ([19721-22-3] in this example) under standard basic conditions to form the thioimidate linked intermediate. Sulfonylation, in this case with tosyl chloride, under referenced conditions (see sulfonate ester above) gives the desired product.

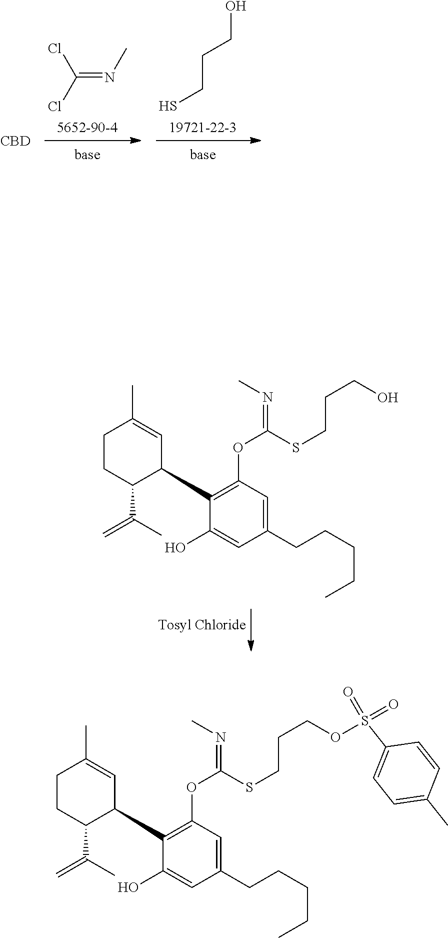

Sulfonate thiophosphinodiamide linked compounds are synthesized as follows. Using conditions similar to those referenced in the epoxide thiophosphinodiamide Scheme, dimethylphosphoramidothioic dichloride ([1498-65-3]) is reacted with a cannabinoid (CBD in this example) and an amino-alcohol ([156-87-6] in this example). Sulfonylation of the adduct, in this case with mesyl chloride, under referenced conditions (see sulfonate ester above) gives the desired product.

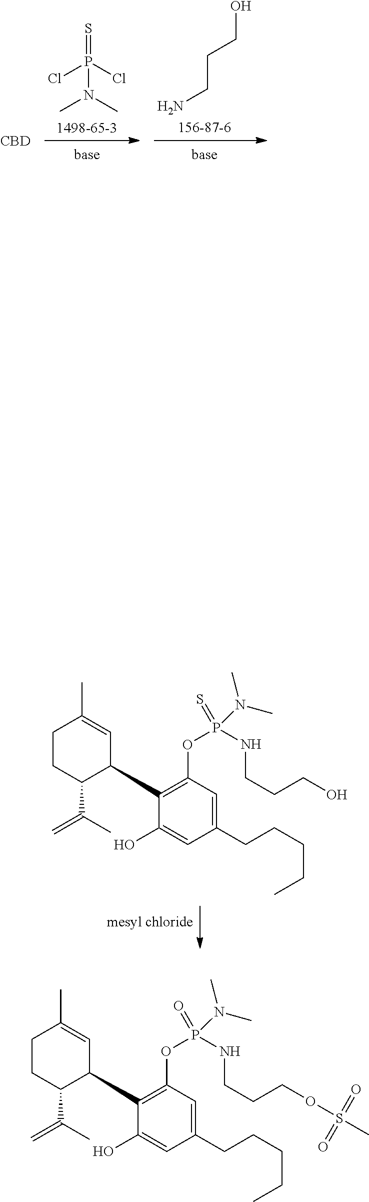

Sulfonate xanthate linked compounds are synthesized as follows. A cannabinoid (CBD in this example) is reacted with thiophosgene (or a suitable thiophosgene surrogate) and a thiol-alcohol ([19721-22-3] in this example) under standard basic conditions to form the xanthate linked intermediate, Sulfonylation, in this case with mesyl chloride, under referenced conditions (see sulfonate ester above) gives the desired product.

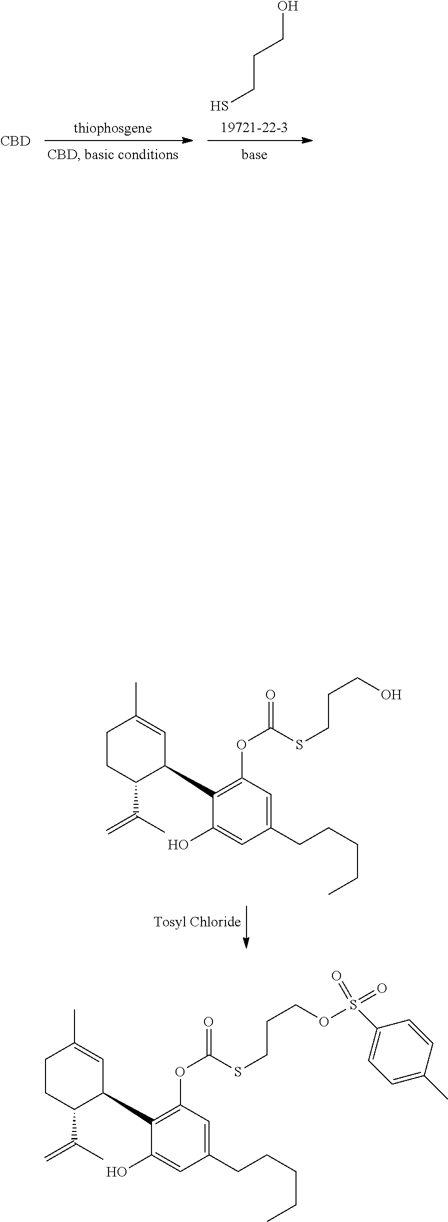 [SMILES: OCCCS ; "CBD" ; C=C(C)[C@@H]1CCC(C)=C[C@H]1c1c(O)cc(CCCCC)cc1OC(=O)SCCCO ; C=C(C)[C@@H]1CCC(C)=C[C@H]1c1c(O)cc(CCCCC)cc1OC(=O)SCCCOS(=O)(=O)c1ccc(C)cc1]

Halide carbamate linked compounds are synthesized as follows. A cannabinoid (CBD in this example) is reacted with phosgene (or a suitable phosgene surrogate) and an aminohalide ([18370-81-5] in this example) under standard basic conditions to form the desired carbamate linked product.

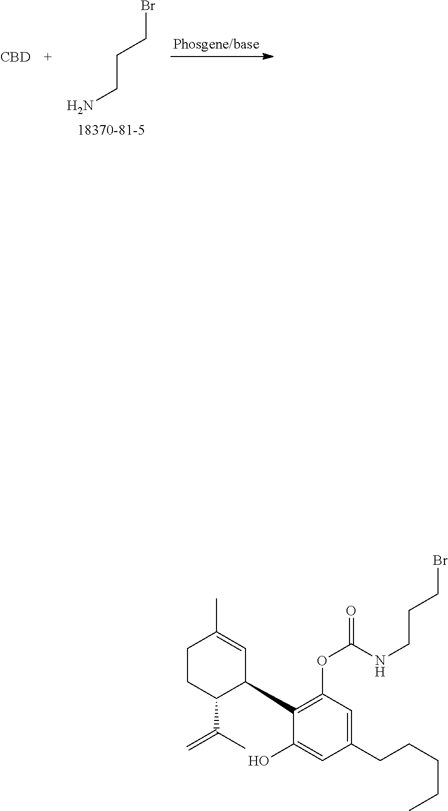 [SMILES: "CBD" ; NCCCBr ; C=C(C)[C@@H]1CCC(C)=C[C@H]1c1c(O)cc(CCCCC)cc1OC(=O)NCCCBr]

Halide carbonate linked compounds are synthesized as follows. A cannabinoid (CBD in this example) is reacted with phosgene (or a suitable phosgene surrogate) and a hydroxyalkyl halide ([627-18-9] in this example) under standard basic conditions to form the desired carbonate linked product.

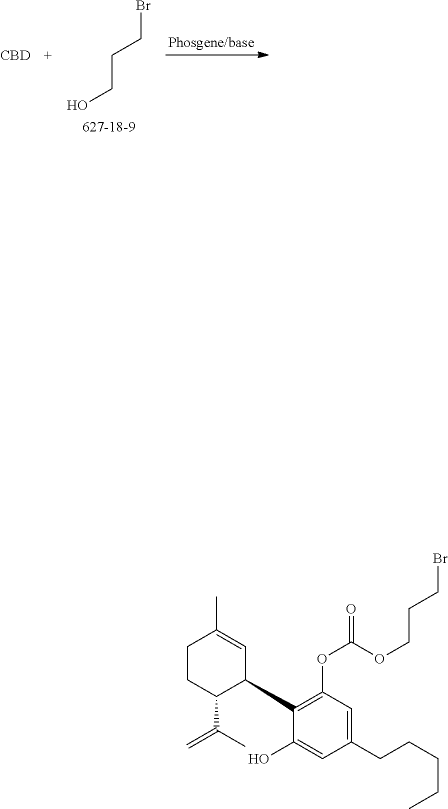 [SMILES: "CBD" ; OCCCBr ; C=C(C)[C@@H]1CCC(C)=C[C@H]1c1c(O)cc(CCCCC)cc1OC(=O)OCCCBr]

Halide ester linked compounds are synthesized as follows. A cannabinoid (CBD in this example) is esterified under standard conditions, in this example with the haloalkyl acid building block [2067-33-6] to give the desired product.

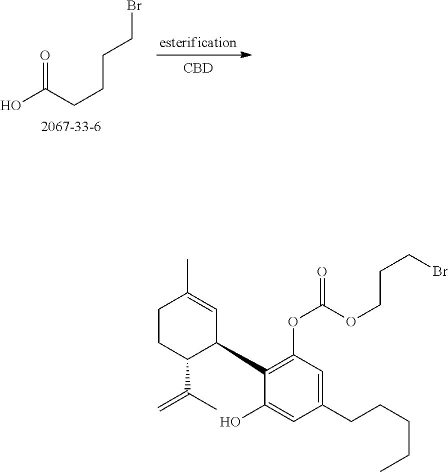

Halide imidate linked compounds are synthesized as follows. A cannabinoid (CBD in this example) is reacted with an imidocarbonyl chloride (in this case [5652-90-4]) and a hydroxyalkyl halide ([627-18-9] in this example) under standard basic conditions to form the desired imidate linked product.

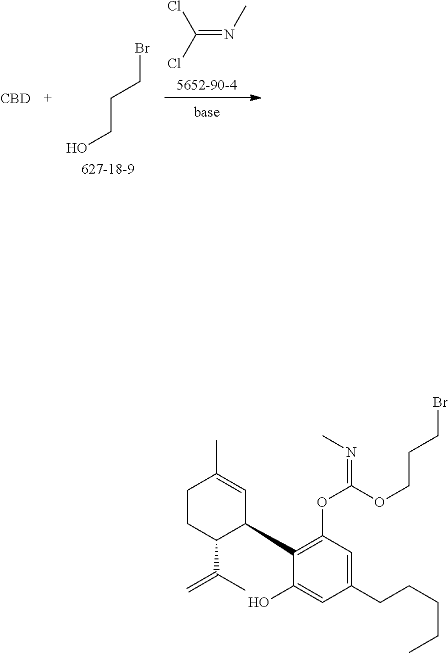

Halide isourea linked compounds are synthesized as follows. A cannabinoid (CBD in this example) is reacted with an imidocarbonyl chloride (in this case [5652-90-4]) and an aminoalkyl halide ([18370-81-5] in this example) under standard basic conditions to form the desired isourea linked product.

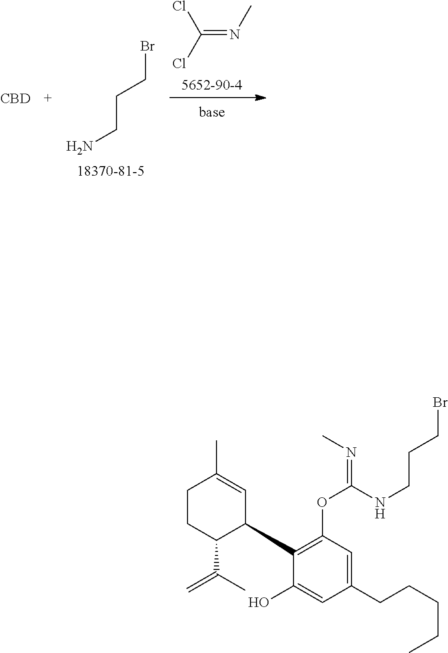

Halide phosphorodiamide linked compounds are synthesized as follows. Using conditions similar to those referenced in the epoxide phosphorodiamide Scheme, N,N-Dimethylphosphoramidodichloridate ([677-43-0]) is reacted with a cannabinoid (CBD in this example) and an aminoalkyl halide ([18370-81-5] in this example) to form the desired product.

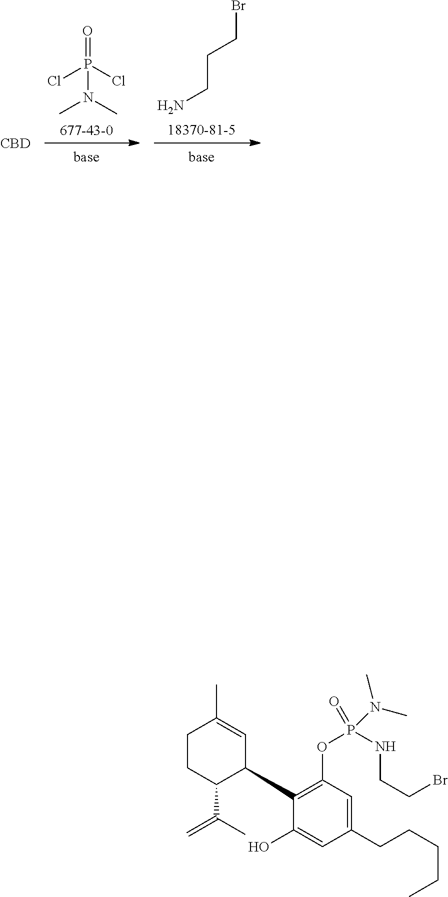

Halide S-alkyl thiocarbonate linked compounds are synthesized as follows. A cannabinoid (CBD in this example) is reacted with phosgene (or a suitable phosgene surrogate) and a haloalkyl thiol ([75694-39-2] in this example) under standard basic conditions, to form the desired S-alkyl thiocarbonate linked product.

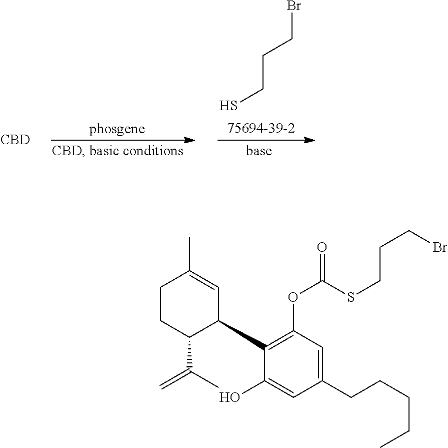 [SMILES: SCCCBr ; "CBD" ; C=C(C)[C@@H]1CCC(C)=C[C@H]1c1c(O)cc(CCCCC)cc1OC(=O)SCCCBr]

Halide thiocarbamate linked compounds are synthesized as follows. A cannabinoid (CBD in this example) is reacted with thiophosgene (or a suitable thiophosgene surrogate) and an aminoalkyl halide ([18370-81-5] in this example) under standard basic conditions to form the desired thiocarbamate linked product.

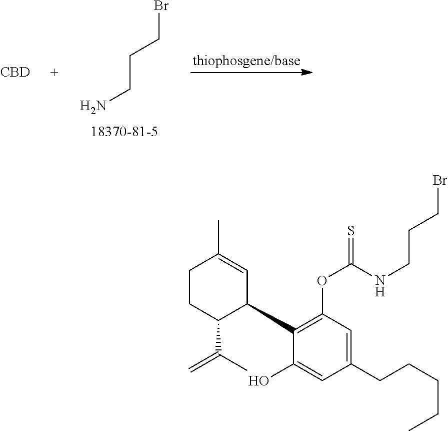 [SMILES: "CBD" ; NCCCBr ; C=C(C)[C@@H]1CCC(C)=C[C@H]1c1c(O)cc(CCCCC)cc1OC(=S)NCCCBr]

Halide thiocarbonate linked compounds are synthesized as follows. A cannabinoid (CBD in this example) is reacted with thiophosgene (or a suitable thiophosgene surrogate) and a hydroxyalkyl halide ([627-18-9] in this example) under standard basic conditions to form the desired thiocarbonate linked product.

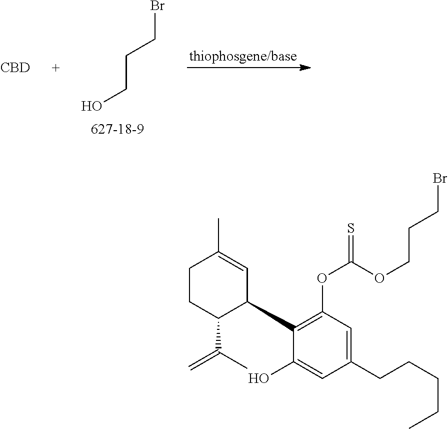 [SMILES: "CBD" ; OCCCBr ; C=C(C)[C@@H]1CCC(C)=C[C@H]1c1c(O)cc(CCCCC)cc1OC(=S)OCCCBr]

Halide thioimidate linked compounds are synthesized as follows. A cannabinoid (CBD in this example) is reacted with an imidocarbonyl chloride (in this case [5652-90-4]) and a haloalkyl thiol ([75694-39-2] in this example) under standard basic conditions to form the desired thioimidate linked product.

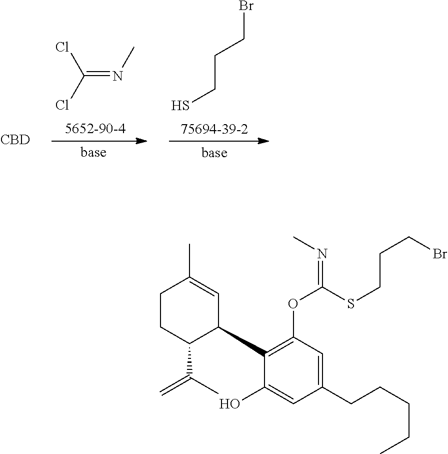

Halide thiophosphinodiamide linked compounds are synthesized as follows. Using conditions similar to those referenced in the epoxide thiophosphinodiamide Scheme, dimethylphosphoramidothioic dichloride ([1498-65-3]) is reacted with a cannabinoid (CBD in this example) and an aminoalkyl halide ([18370-81-5] in this example) to form the desired product.

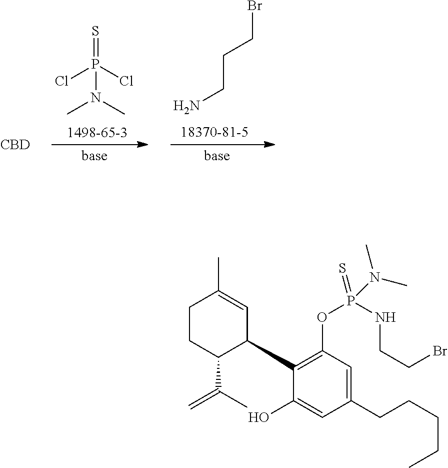

Halide xanthate linked compounds are synthesized as follows. A cannabinoid (CBD in this example) is reacted with thiophosgene (or a suitable thiophosgene surrogate) and a haloalkyl thiol ([75694-39-2] in this example) under standard basic conditions to form the desired xanthate linked product.

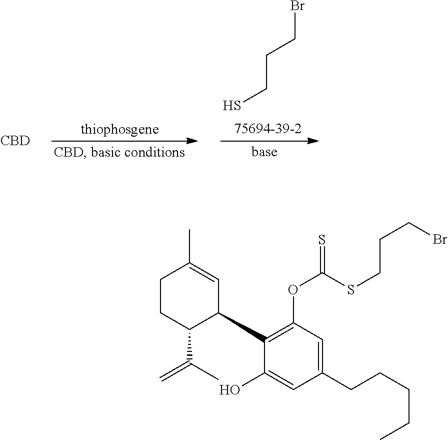 [SMILES: SCCCBr ; "CBD" ; C=C(C)[C@@H]1CCC(C)=C[C@H]1c1c(O)cc(CCCCC)cc1OC(=S)SCCCBr]

Compounds linked to the temozolomide component are synthesized as follows. The iodo acid [7425-27-6] is reacted with a cannabinoid (CBD) under standard esterification conditions to give the iodo ester intermediate. Following conditions (see Scheme) similar to those published for the synthesis of temozolomide from iodomethane, the desired compound is produced by N-alkylation of [108030-65-5].

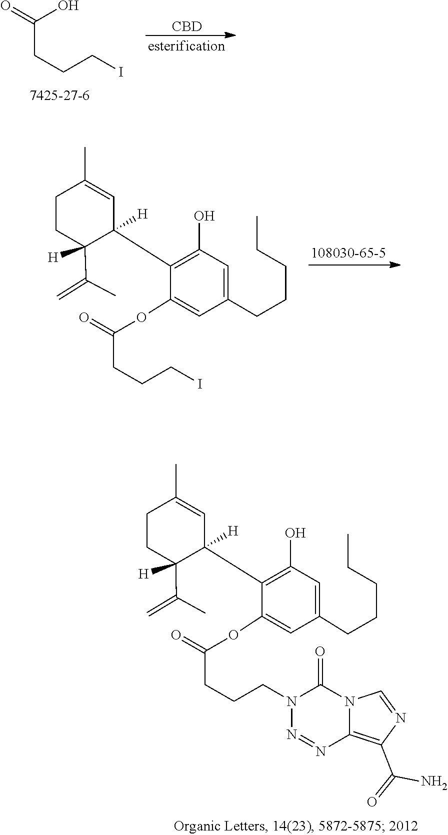 [SMILES: O=C(O)CCCI ; "CBD" ; C=C(C)[C@@H]1CCC(C)=C[C@H]1c1c(O)cc(CCCCC)cc1OC(=O)CCCI ; C=C(C)[C@@H]1CCC(C)=C[C@H]1c1c(O)cc(CCCCC)cc1OC(=O)CCCn1nnc2c(C(N)=O)ncn2c1=O]

Ester compounds linked to the 5-fluorouracil component at the 1-position are synthesized as follows. The known building block [6214-60-4] is reacted with a cannabinoid (CBD) under standard esterification conditions to give the product.

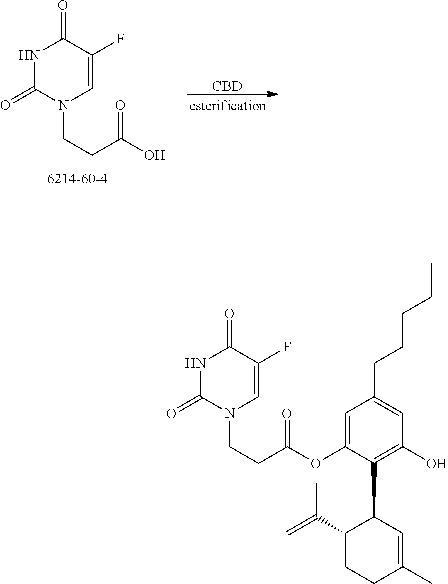 [SMILES: O=C(O)CCn1cc(F)c(=O)[nH]c1=O ; "CBD" ; C=C(C)[C@H]1CCC(C)=C[C@@H]1c1c(O)cc(CCCCC)cc1OC(=O)CCn1cc(F)c(=O)[nH]c1=O]

Carbonate compounds linked to the 5-fluorouracil component at the 1-position are synthesized as follows. The building block [106206-99-9] is reacted with phosgene (or a suitable surrogate) and CBD under standard basic conditions to give the product.

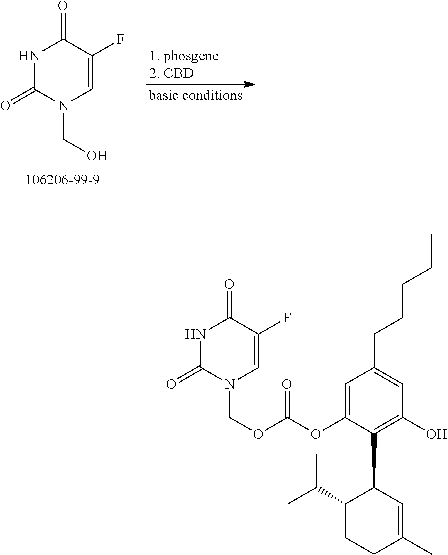 [SMILES: O=c1[nH]c(=O)n(CO)cc1F ; CCCCCc1cc(O)c([C@H]2C=C(C)CC[C@@H]2C(C)C)c(OC(=O)OCn2cc(F)c(=O)[nH]c2=O)c1]

Carbamate compounds linked to the 5-fluorouracil component at the 1-position are synthesized as follows. The building block [1339797-10-2] is reacted with phosgene (or a suitable surrogate) and CBD under standard basic conditions to give the product

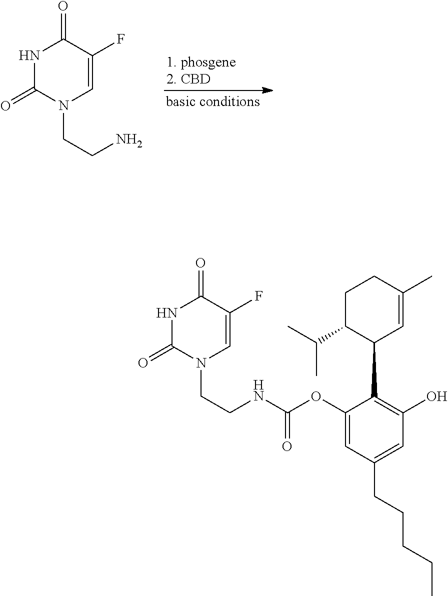 [SMILES: NCCn1cc(F)c(=O)[nH]c1=O ; CCCCCc1cc(O)c([C@@H]2C=C(C)CC[C@H]2C(C)C)c(OC(=O)NCCn2cc(F)c(=O)[nH]c2=O)c1]

Ester compounds linked to the 5-fluorouracil component at the 3-position are synthesized as follows. The known building block [905265-53-4] is reacted with a cannabinoid (CBD) under standard esterification conditions to give the product.

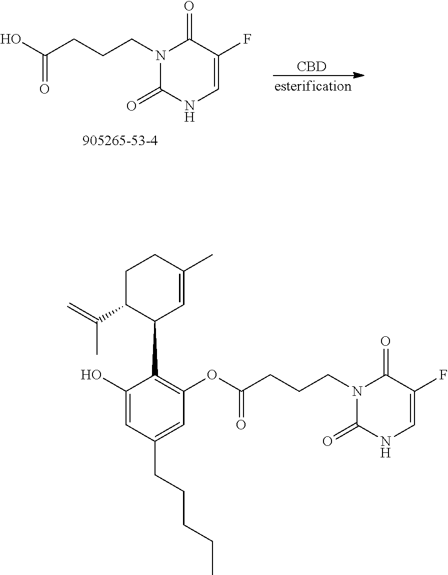

Carbonate compounds linked to the 5-fluorouracil component at the 3-position are synthesized as follows. The building block [948036-30-4] is reacted with phosgene (or a suitable surrogate) and CBD under standard basic conditions to give the product.

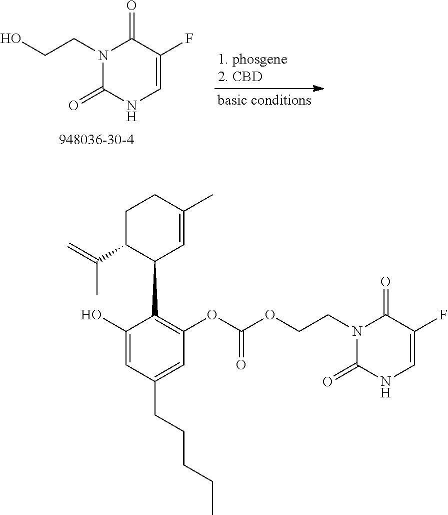 [SMILES: O=c1[nH]cc(F)c(=O)n1CCO ; C=C(C)[C@@H]1CCC(C)=C[C@H]1c1c(O)cc(CCCCC)cc1OC(=O)OCCn1c(=O)[nH]cc(F)c1=O]

1. A conjugate molecule, or a pharmaceutically acceptable salt thereof, comprising a first therapeutic agent component and a first cannabinoid component, wherein the first therapeutic agent component is covalently attached, either directly or via a linker to a first linker, to the first cannabinoid component; and wherein:
(A) the first therapeutic agent component is selected from the group consisting of: 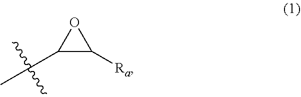
wherein Rₐ is absent or is C1-C3 linear or branched alkyl or C1-C3 linear or branched heteroalkyl comprising an O, N, or S atom; 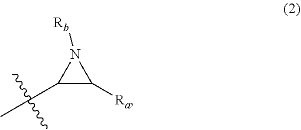
wherein Rₐ is as defined above and R_{b} is R or —PS(NR_{c1}R_{c2}), wherein R_{c1} and R_{c2} independently are C1-C6 linear or branched alkyl or C1-C6 cycloalkyl, and wherein R is selected from the group consisting of:
(a) H;
(b) C1-C8 linear or branched alkyl, optionally substituted with
(1) 1, 2, 3, 4, 5, 6, 7, 8, or 9 fluorine atoms; and/or
(2) 1, 2, or 3 substituents independently selected from the Group One Substituents;
(c) C1-C8 linear or branched heteroalkyl containing 1, 2, or 3 heteroatoms independently selected from O, N, and S and optionally substituted with
(1) 1, 2, 3, 4, 5, 6, 7, 8, or 9 fluorine atoms; and/or
(2) 1, 2, or 3 substituents independently selected from the Group One Substituents;
(d) phenyl, optionally substituted with 1, 2, or 3 substituents independently selected from the group consisting of:
(1) C1-C6 linear or branched alkyl, optionally substituted with
(i) 1, 2, 3, 4, 5, or 6 fluorine atoms; and/or
(ii) 1 or 2 substituents independently selected from the Group Two Substituents; and
(2) C1-C6 linear or branched heteroalkyl containing 1 or 2 heteroatoms independently selected from O, N, and S and optionally substituted with
(i) 1, 2, 3, 4, 5, or 6 fluorine atoms; and/or
(ii) 1 or 2 substituents independently selected from the Group One Substituents;
(e) a 6- to 10-membered aromatic, optionally substituted with 1, 2, 3, or 4 substituents independently selected from the group consisting of:
(1) phenyl;
(2) halide;
(3) cyano;
(4) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents, and
(5) C1-C6 linear or branched heteroalkyl containing 1, 2, or 3 atoms independently selected from O, N, and S and optionally substituted with
(i) 1, 2, 3, 4, 5, 6, 7, 8, or 9 fluorine atoms; and/or
(ii) 1, 2, or 3 substituents independently selected from the Group Two Substituents;
(f) 5- to 10-membered heteroaromatic comprising 1, 2, 3, 4, 5, or 6 heteroatoms independently selected from O, N, and S and optionally substituted with 1, 2, 3, or 4 substituents independently selected from
(1) phenyl;
(2) halide;
(3) cyano;
(4) trifluoromethyl;
(5) C1-C6 linear or branched alkyl optionally substituted with
(i) 1, 2, 3, 4, 5, 6, 7, 8, or 9 fluorine atoms; and/or
(ii) 1, 2, or 3 substituents independently selected from the Group Two Substituents; and
(6) C1-C6 linear or branched heteroalkyl containing 1, 2, or 3 atoms independently selected from O, N, and S and optionally substituted with
(i) 1, 2, 3, 4, 5, 6, 7, 8, or 9 fluorine atoms; and/or
(ii) 1, 2, or 3 substituents independently selected from the Group Two Substituents;
(g) 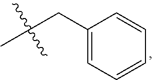 [SMILES: *Cc1ccccc1]
optionally substituted with 1, 2, or 3 substituents independently selected from the group consisting of:
(1) C1-C6 linear or branched alkyl, optionally substituted with
(i) 1, 2, 3, 4, 5, or 6 fluorine atoms; and/or
(ii) 1, 2, or 3 substituents independently selected from the Group Two Substituents;
(h) 3- to 9-membered cycloheteroalkyl having 1, 2, or 3 heteroatoms independently selected from O, N, and S and optionally substituted with 1, 2, or 3 substituents independently selected from the group consisting of:
(1) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents,
(2) C1-C6 linear or branched heteroalkyl, optionally substituted with
(i) 1, 2, 3, 4, 5, 6, 7, 8, or 9 fluorine atoms and/or
(ii) 1, 2, or 3 substituents independently selected from the Group Two Substituents,
(3) phenyl, optionally substituted with 1, 2, or 3 substituents independently selected from the Group Two Substituents, and
(4) 5- to 10-membered heteroaromatic, optionally substituted with 1, 2, or 3 substituents independently selected from the Group Two Substituents; and
(i) C3-C6 cycloalkyl, optionally substituted with 1, 2, or 3 substituents independently selected from:
(1) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents,
(2) C1-C6 linear or branched heteroalkyl, optionally substituted with
(i) 1, 2, 3, 4, 5, 6, 7, 8, or 9 fluorine atoms; and/or
(ii) 1, 2, or 3 substituents independently selected from the Group Two Substituents,
(3) phenyl, optionally substituted with 1, 2, or 3 substituents independently selected from Group Two Substituents; and
(4) 5- to 10-membered heteroaromatic, optionally substituted with 1, 2, or 3 substituents independently selected from the Group Two Substituents;
Group One Substituents is a group of substituents consisting of:
(a) —OH;
(b) —NH₂;
(c) ═O;
(d) ═S;
(e) ═NR₇, where R₇ is H or is C1-C3 linear or branched alkyl or C1-C3 linear or branched heteroalkyl comprising an O, N, or S atom;
(f) —C(O)OR₄, wherein R₄ is H or C1-C3 linear or branched alkyl;
(g) —C(O)NR₅R₆, wherein R₅ and R₆ independently are H or C1-C6 linear or branched alkyl;
(h) halide;
(i) C1-C6 linear or branched alkoxyl;
(j) C1-C6 linear or branched alkylamino;
(k) C1-C6 linear or branched dialkylamino;
(l) 6- to 10-membered aromatic, optionally substituted with 1, 2, 3, or 4 substituents independently selected from
(i) phenyl;
(ii) halide;
(iii) cyano;
(iv) C1-C6 linear or branched alkyl, optionally substituted with
(1) 1, 2, 3, 4, 5, 6, 7, 8, or 9 fluorine atoms; and/or
(2) 1, 2, or 3 substituents independently selected from the Group Two Substituents; and
(v) C1-C6 linear or branched heteroalkyl containing 1, 2, or 3 atoms independently selected from O, N, and S and optionally substituted with
(1) 1, 2, 3, 4, 5, 6, 7, 8, or 9 fluorine atoms; and/or
(2) 1, 2, or 3 substituents independently selected from the Group Two Substituents;
(m) 5- to 10-membered heteroaromatic, optionally substituted with 1, 2, 3, or 4 substituents independently selected from
(i) phenyl;
(ii) halide;
(iii) cyano;
(iv) C1-C6 linear or branched alkyl, optionally substituted with
(1) 1, 2, 3, 4, 5, 6, 7, 8, or 9 fluorine atoms; and/or
(2) 1, 2, or 3 substituents independently selected from the Group Two Substituents; and
(v) C1-C6 linear or branched heteroalkyl containing 1, 2, or 3 atoms independently selected from O, N, and S and optionally substituted with
(1) 1, 2, 3, 4, 5, 6, 7, 8, or 9 fluorine atoms; and/or
(2) 1, 2, or 3 substituents independently selected from the Group Two Substituents;
(n) 3- to 9-membered cycloheteroalkyl having 1, 2, or 3 heteroatoms independently selected from O, N, and S, optionally substituted with 1, 2, 3, or 4 substituents independently selected from
(i) phenyl;
(ii) halide;
(iii) cyano;
(iv) C1-C6 linear or branched alkyl, optionally substituted with
(1) 1, 2, 3, 4, 5, 6, 7, 8, or 9 fluorine atoms; and/or
(2) 1, 2, or 3 substituents independently selected from the Group Two Substituents; and
(v) C1-C6 linear or branched heteroalkyl containing 1, 2, or 3 atoms independently selected from O, N, and S and optionally substituted with
(1) 1, 2, 3, 4, 5, 6, 7, 8, or 9 fluorine atoms; and/or
(2) 1, 2, or 3 substituents independently selected from the Group Two Substituents; and
(o) C3-C6 cycloalkyl, optionally substituted with 1, 2, 3, or 4 substituents independently selected from
(i) phenyl;
(ii) halide;
(iii) cyano;
(iv) C1-C6 linear or branched alkyl, optionally substituted with
(1) 1, 2, 3, 4, 5, 6, 7, 8, or 9 fluorine atoms; and/or
(2) 1, 2, or 3 substituents independently selected from the Group Two Substituents; and
(v) C1-C6 linear or branched heteroalkyl containing 1, 2, or 3 atoms independently selected from O, N, and S and optionally substituted with
(1) 1, 2, 3, 4, 5, 6, 7, 8, or 9 fluorine atoms; and/or
(2) 1, 2, or 3 substituents independently selected from the Group Two Substituents;
Group Two Substituents is a group of substituents consisting of:
(a) —OH;
(b) —NH₂;
(c) ═O;
(d) ═S;
(e) ═NR₇, where R₇ is H or is C1-C3 linear or branched alkyl or C1-C3 linear or branched heteroalkyl comprising an O, N, or S atom;
(f) —C(O)OR₄, wherein R₄ is H or C1-C3 linear or branched alkyl;
(g) —C(O)NR₅R₆, wherein R₅ and R₆ independently are H or C1-C6 linear or branched alkyl;
(h) halide;
(i) cyano;
(j) trifluoromethyl;
(k) C1-C6 linear or branched alkoxyl;
(l) C1-C6 linear or branched alkylamino;
(m) C1-C6 linear or branched dialkylamino;
(n) 6- to 10-membered aromatic; and
(o) 5- to 10-membered heteroaromatic comprising 1, 2, 3, 4, 5, or 6 heteroatoms independently selected from O, N, and S; 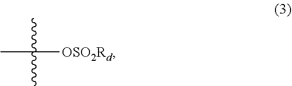
wherein R_{d} is
(a) C1-C8 linear or branched alkyl, optionally substituted with
(i) 1, 2, 3, 4, 5, 6, 7, 8, or 9 fluorine atoms; and/or
(ii) 1, 2, or 3 substituents independently selected from the Group One Substituents; or
(b) phenyl, optionally substituted with 1, 2, or 3 substituents independently selected from the group consisting of C1-C6 linear or branched alkyl, optionally substituted with
(i) 1, 2, 3, 4, 5, or 6 fluorine atoms; and/or
(ii) 1 or 2 substituents independently selected from the Group Two Substituents; 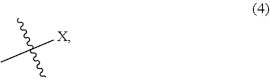
wherein X is Cl, Br, or I; 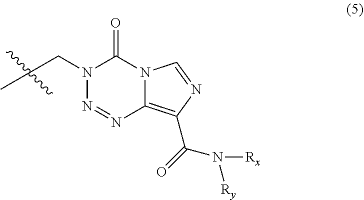
wherein Rₓ and R_{y} independently are H or C1-C3 linear or branched alkyl; 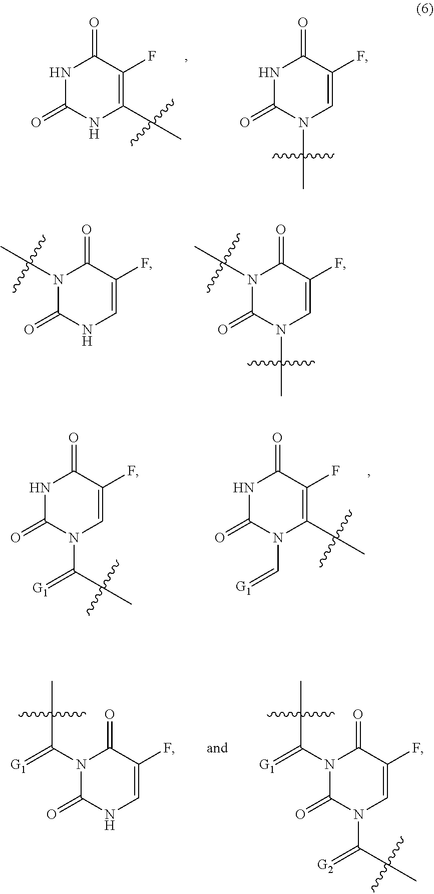
wherein G₁ and G₂ independently are selected from the group consisting of O, S, and NR; and
(7) a therapeutic agent component selected from the group consisting of a diclofenac component, a celecoxib component, a gemcitabine component, an entecavir component, an emtricitabine component, an axitinib component, a batimastat component, a bosutinib component, a crizotinib component, an erlotinib component, a gefitinib component, an erlotinib component, an everolimus component, a temsirolimus component, a ganetespib component, a glasdeib component, an imatinib component, a lapatinib component, a navitoclax component, a nilotinib component, a pazopanib component, a component, a luminespib component, an obatoclax component, a ruxolitinib component, a saridegib component, a sunitinib component, a trametinib component, a warfarin component, a daclatasvir component, an etoposide component, an atazanavir component, a pravastatin component, a dasatinib component, a didanosine component, and a stavudine component; and
(B) the first linker is selected from the group consisting of: 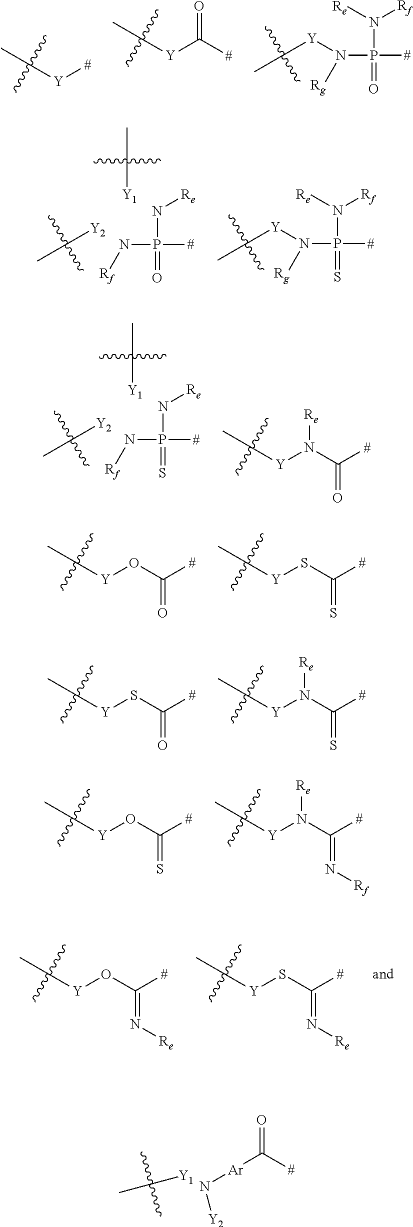
in which [symbol] marks a bond attaching the Type (Ib) linker to the therapeutic agent component, # indicates a site of covalent attachment to the cannabinoid component, and in which:
Y, Y₁, and Y₂ independently are absent or Y, Y₁, and Y₂ independently are selected from the group consisting of:
(a) C1-C12 linear or branched alkyl, optionally substituted with
(1) 1, 2, 3, 4, 5, 6, 7, 8, or 9 fluorine atoms; and/or
(2) 1, 2, or 3 substituents selected from the Group One Substituents;
(b) C2-C12 linear or branched alkenyl, optionally substituted with
(1) 1, 2, 3, 4, 5, 6, 7, 8, or 9 fluorine atoms; and/or
(2) 1, 2, or 3 substituents selected from the Group One Substituents;
(c) C1-C12 linear or branched heteroalkyl containing 1, 2, 3, or 4 heteroatoms independently selected from O, N, and S, optionally substituted with
(1) 1, 2, 3, 4, 5, 6, 7, 8, or 9 fluorine atoms; and/or
(2) 1, 2, or 3 substituents selected from the Group One Substituents;
(d) a 6- to 10-membered aromatic, optionally substituted with 1, 2, 3, or 4 substituents independently selected from the group consisting of:
(1) phenyl,
(2) halide,
(3) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents, and
(4) C1-C6 linear or branched heteroalkyl containing 1, 2, or 3 atoms independently selected from O, N, and S and optionally substituted with
(i) 1, 2, 3, 4, 5, 6, 7, 8, or 9 fluorine atoms; and/or
(ii) 1, 2, or 3 substituents independently selected from the Group Two Substituents;
(e) a 6- to 10-membered heteroaromatic comprising 1, 2, 3, 4, 5, or 6 heteroatoms independently selected from O, N, and S and optionally substituted with 1, 2, 3, or 4 substituents independently selected from
(1) phenyl,
(2) halide,
(3) trifluoromethyl,
(4) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents, and
(5) C1-C6 linear or branched heteroalkyl containing 1, 2, or 3 atoms independently selected from O, N, and S and optionally substituted with
(i) 1, 2, 3, 4, 5, 6, 7, 8, or 9 fluorine atoms; and/or
(ii) 1, 2, or 3 substituents independently selected from the Group Two Substituents; and
(f) a C1-C24 linear or branched heteroalkyl containing 1, 2, 3, 4, 5, 6, 7, or 8 heteroatoms independently selected from O, N, and S, optionally substituted with
(i) 1, 2, 3, 4, 5, 6, 7, 8, or 9 fluorine atoms; and/or
(ii) 1, 2, 3, 4, 5, or 6 substituents selected from the Group One Substituents; and
(2) R₁, R₂, and R₃ independently are R as defined above.

2. The conjugate molecule of claim 1, wherein the first therapeutic agent component is covalently attached to the first cannabinoid component via a first linker.

3. The conjugate molecule of claim 1, wherein the first linker is covalently attached to a first carboxylic acid group of the first cannabinoid component.

4. The conjugate molecule of claim 2, wherein the first therapeutic agent component is covalently attached to a first hydroxy group of the first cannabinoid component.

5. The conjugate molecule of claim 1, wherein the first therapeutic agent component is covalently attached to a first carboxylic acid group of the first cannabinoid component.

6. The conjugate molecule of claim 1, wherein the first therapeutic agent component is covalently attached to a first hydroxy group of the first cannabinoid component.

7. The conjugate molecule of claim 1, further comprising a second therapeutic agent component covalently attached to the first cannabinoid component.

8. The conjugate molecule of claim 7, wherein the second therapeutic agent component is:
(a) covalently attached to a first hydroxy group of the first cannabinoid component;
(b) covalently attached to a second hydroxy group of the first cannabinoid component;
(c) covalently attached to a first carboxylic acid group of the first cannabinoid component;
(d) covalently attached to a second carboxylic acid group of the first cannabinoid component;
(e) covalently attached to the first cannabinoid component via a second linker to a first hydroxy group of the first cannabinoid component;
(f) covalently attached to the first cannabinoid component via a second linker to a second hydroxy group of the first cannabinoid component;
(g) covalently attached to the first cannabinoid component via a second linker to a first carboxylic acid group of the first cannabinoid component; or
(h) covalently attached to the first cannabinoid component via a second linker to a second carboxylic acid group of the first cannabinoid component.

9. The conjugate molecule of claim 5, wherein the second therapeutic agent component is 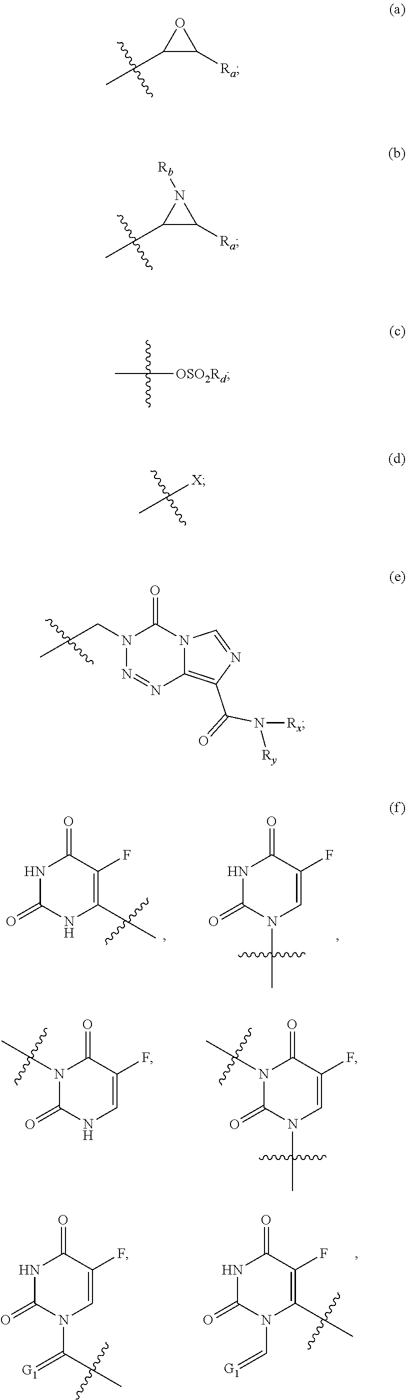 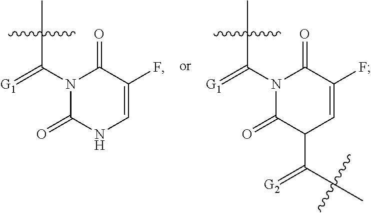
or
(g) wherein the second therapeutic agent component is selected from the group consisting of a therapeutic agent component selected from the group consisting of a diclofenac component, a celecoxib component, a gemcitabine component, an entecavir component, an emtricitabine component, an axitinib component, a batimastat component, a bosutinib component, a crizotinib component, an erlotinib component, a gefitinib component, an erlotinib component, an everolimus component, a temsirolimus component, a ganetespib component, a glasdeib component, an imatinib component, a lapatinib component, a navitoclax component, a nilotinib component, a pazopanib component, a component, a luminespib component, an obatoclax component, a ruxolitinib component, a saridegib component, a sunitinib component, a trametinib component, a warfarin component, a daclatasvir component, an etoposide component, an atazanavir component, a pravastatin component, a dasatinib component, a didanosine component, and a stavudine component.

10-15. (canceled)

16. The conjugate molecule of claim 1, wherein first and second linkers independently are selected from the group consisting of:

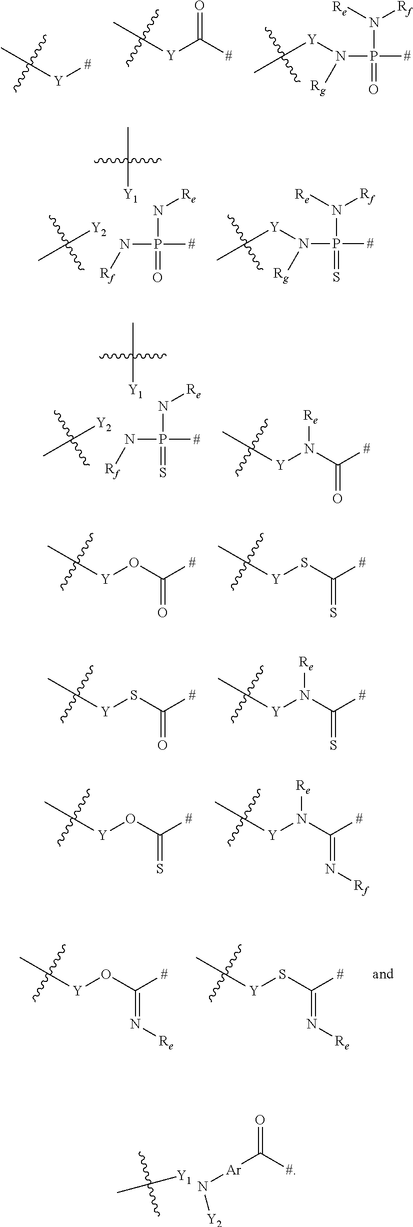

17. The conjugate molecule of claim 16, wherein the first linker is selected from the group consisting of

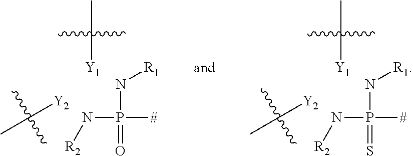

18. The conjugate molecule of claim 16, wherein the first therapeutic agent component is
(a) covalently attached at Y₁;
(b) covalently attached at Y₂;
(c) covalently attached at Y₁ and wherein a further therapeutic agent component is covalently attached at Y₂; or
(d) covalently attached at Y₁ and wherein a further therapeutic agent component is covalently attached at Y₂ and wherein the further therapeutic agent component is selected from the group consisting of: 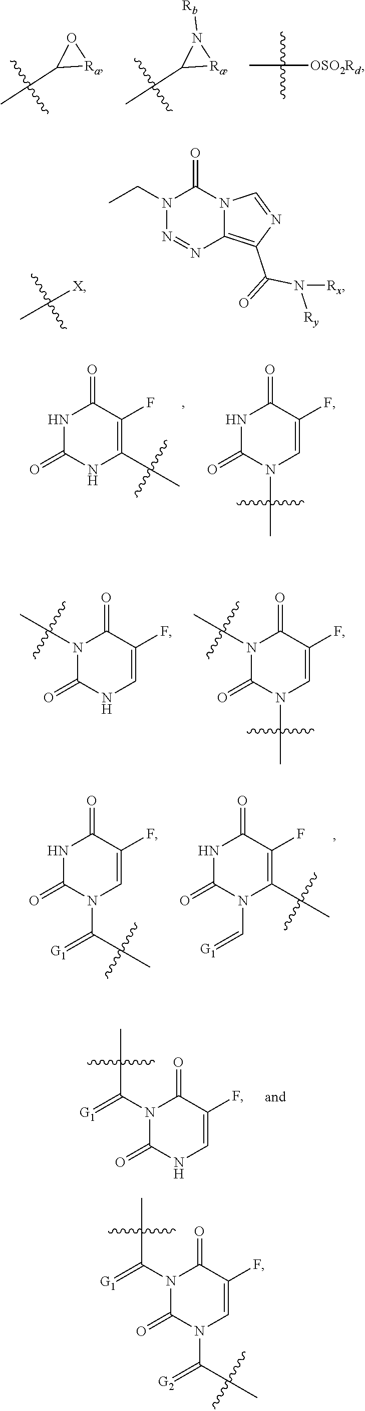
and a therapeutic agent component selected from the group consisting of a diclofenac component, a celecoxib component, a gemcitabine component, an entecavir component, an emtricitabine component, an axitinib component, a batimastat component, a bosutinib component, a crizotinib component, an erlotinib component, a gefitinib component, an erlotinib component, an everolimus component, a temsirolimus component, a ganetespib component, a glasdeib component, an imatinib component, a lapatinib component, a navitoclax component, a nilotinib component, a pazopanib component, a component, a luminespib component, an obatoclax component, a ruxolitinib component, a saridegib component, a sunitinib component, a trametinib component, a warfarin component, a daclatasvir component, an etoposide component, an atazanavir component, a pravastatin component, a dasatinib component, a didanosine component, and a stavudine component.

19-21. (canceled)

22. The conjugate molecule of claim 1, wherein the first therapeutic agent component is 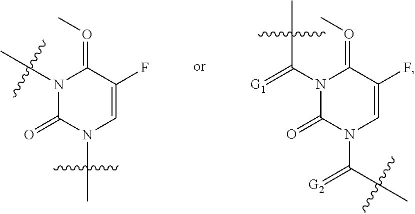
wherein the conjugate molecule further comprises a second cannabinoid component.

23. The conjugate molecule of claim 1, wherein:
(a) the first cannabinoid component is provided by a cannabinoid selected from the group consisting of a cannabigerol, a cannabichromene, a cannabidiol, a tetrahydrocannabinol, a cannabicyclol, a cannabielsoin, a cannabinol, a cannabinodiol, a cannabitriol, a dehydrocannabifuran, a cannabifuran, a cannabichromanon, and a cannabiripsol, or an active metabolite thereof; or
(b) the first cannabinoid component is a cannabidiol component.

24. (canceled)

25. The conjugate molecule of claim 22, wherein:
(a) the second cannabinoid component is provided by a cannabinoid selected from the group consisting of a cannabigerol, a cannabichromene, a cannabidiol, a tetrahydrocannabinol, a cannabicyclol, a cannabielsoin, a cannabinol, a cannabinodiol, a cannabitriol, a dehydrocannabifuran, a cannabifuran, a cannabichromanon, and a cannabiripsol, or an active metabolite thereof; or
(b) the second cannabinoid component is a cannabidiol component.

26. (canceled)

27. (canceled)

28. A pharmaceutical composition comprising a conjugate molecule of claim 1, or a pharmaceutically acceptable salt thereof, and a pharmaceutically acceptable vehicle.

29. The pharmaceutical composition of claim 28, which:
(a) comprises a racemic mixture of conjugate molecules
(b) comprises a single enantiomer of the conjugate molecule;
(c) comprises a mixture of diastereomers of the conjugate molecule;
(d) comprises a mixture of double bond isomers of the conjugate molecule;
(e) comprises a Z-double bond isomer of the conjugate molecule;
(f) comprises a E-double bond isomer of the conjugate molecule; or
(g) comprises an isotopic variant of the conjugate molecule.

30-35. (canceled)

36. A method of treating a hyperproliferative disorder, comprising administering to a patient in need thereof a conjugate molecule of claim 1 or a pharmaceutically acceptable salt thereof.

37. The method of claim 36, wherein the hyperproliferative disorder is a cancer.

38. The method of claim 38, wherein the conjugate molecule is administered in conjunction with a second cancer therapy.
